(19) United States
(12) Patent Application Publication
(10) Pub. No.: US 2022/0298468 A1
(43) Pub. Date: Sep. 22, 2022

(54) A METHOD FOR DEVELOPMENTALLY ACTIVATING A CELL

(71) Applicants: Christopher B REID, Arlington, TX (US); Melissa BRAGA, Monterey Park, CA (US); Lillian N SANTAMARIA, Los Angeles, CA (US); Ashley N WICKREMA, Los Angeles, CA (US); Marinne D WICKREMA, Los Angeles, CA (US); Anthony HAO DINH, Garden Grove, CA (US); Yaman EKSIOGLU, Atlanta, GA (US); Mat HOANG HO, Birmingham, AL (US)

(72) Inventors: Christopher B REID, Arlington, TX (US); Melissa BRAGA, Monterey Park, CA (US); Lillian N SANTAMARIA, Los Angeles, CA (US); Ashley N WICKREMA, Los Angeles, CA (US); Marinne D WICKREMA, Los Angeles, CA (US); Anthony HAO DINH, Garden Grove, CA (US); Yaman EKSIOGLU, Atlanta, GA (US); Mat HOANG HO, Birmingham, AL (US)

(21) Appl. No.: 17/763,623

(22) PCT Filed: Sep. 24, 2020
(86) PCT No.: PCT/US2020/052378

§ 371 Date: Mar. 24, 2022

Related U.S. Application Data
(63) Continuation of application No. 16/579,889, filed on Sep. 24, 2019.

(51) Int. Cl. C12M 1/00; C12M 1/42; C12N 15/11; C12M 3/00; C12N 15/10; A61K 48/00

(52) U.S. Cl. CPC C12M 43/00; C12M 21/00; C12M 35/02; C12N 15/111; C12M 23/42; C12N 15/102; C12N 15/1031; C12N 15/1024; A61K 48/0058; C12Q 1/6811

(57) ABSTRACT

The claimed invention is directed towards equipment, methods and compositions involving application of an Electric field that are suitable for in vivo electroporation, in vitro application of an Electric field and the generation of developmentally-activated, totipotent, pluripotent, pluripotent-like, multipotent, and/or self-renewing cells which are capable of beginning to differentiate in culture into a variety of cell types and capable of further differentiation in vivo. The claimed invention is also directed towards the generation of desirable, differentiating somatic cell populations transplantable to animals or patients, to drug screening and drug discovery, cellular therapy, immunotherapy, gene therapy, tissue engineering, and the treatment of patients suffering from diseases that may be ameliorated by these methods. This invention also provides methods for preventing, treating, or retarding disease, for example, immunodeficiency virus (e.g. HIV-1, HIV-2, SIV, FIV, etc.) infection.

The present PCT application claims priority to U.S. patent application Ser. No. 16/579,889 filed on 24 Sep. 2019, which is hereby incorporated herein by reference in its entirety and made part of the present PCT application for all purposes.

### FIELD OF THE INVENTION

This invention relates to methods, compositions and equipment for developmental activation of cells to desired cell types and uses of said cells in industry and biomedical applications.

### BACKGROUND OF THE INVENTION

Current methods of obtaining pluripotent cells generally rely on introduction of exogenous nucleic acids or inefficient methods involving only small molecules. U.S. Pat. No. 3,598,119 discloses an injection needle that may be guided through an inner lumen of a catheter for insertion of the needle under skin and a bladder at the distal end of a catheter—that may be inflated through another lumen so as to fix the position of the point of the needle.

U.S. Pat. No. 4,578,061 discloses a catheter featuring an injection needle which is longitudinally movable beyond the distal end of the catheter. A dual chamber system provides for movement of a plunger to extend the injection needle and apply a predetermined dose of injectant.

U.S. Pat. No. 4,578,061 discloses an injection catheter having a longitudinally_movable needle which may be extended_through a lumen and out of the side wall of the catheter for injecting-a blood vessel. The needle is normally retracted into the device—in preparation for deployment.

U.S. Pat. No. 5,244,460 is directed toward a method for inserting a catheter into a coronary artery and injecting into the target tissue, organ or cavity a blood vessel growth promoting peptide via the port of the catheter.

U.S. Pat. No. 5,419,777 is directed toward an injection needle which protrudes laterally through the side walls of the distal end of the catheter.

U.S. Pat. No. 5,431,168_is directed toward a steerable catheter which includes a puller wire for controlling the distal end of the catheter from a control handle which is mounted on the proximal end of the catheter.

U.S. patent application Ser. No. 09/019,453-discloses an injection catheter system for infusing a diagnostic or therapeutic agent into the wall of an organ which includes an electromagnetic sensor disposed within the distal end of the catheter.

### SUMMARY OF THE INVENTION

The present invention relates to equipment and apparatuses and methods suitable for developmentally-activating a cell thereby enabling i. repair or renewal, etc. of a cell, tissue or organ, ii. correction of a genetic abnormality iii. protein therapy, iv. tissue engineering, v. and delivery of a cellular cargo. In one aspect, the desired cell type is generated by application of energy in the form of a regulated electrical field (e.g. magnetporation). In another aspect, cells are exposed to an Electric field using electroporation. Magnetoporation, which relies on generation of a magnetic field (Liu et al. 2012; Novickij et. al., 2013; Du et al., 2018; Staigvila, et al 2019) and electroporation are both effective in porating a cell membrane and producing cellular uptake of a molecule or compound or element. The present invention teaches in part that treatment of cells by application of an Electrical field, including by use of electroporation and magnetoporation, can also developmentally activate a cell in conjunction with proteins and small molecules promoting the desired cell type. Cells successfully treated in this manner have a variety of uses. Among other uses, the successfully treated cells are capable of carrying a wide variety of cellular cargoes including biological and non-biological cargoes, and including organic and non-organic cargoes, including synthetic and natural cargoes, and including large or small molecules, elements and compounds. In many embodiments, a cellular cargo is loaded into cells by application of an Electric field (e.g. by magnetoporation or electroporation) or other suitable methods known to the art. In some embodiments, the cargo is loaded for subsequent delivery to a tissue, organ, or patient in need thereof.

### BRIEF DESCRIPTION OF THE DRAWINGS

FIG. 1 depicts a three-dimensional, Principal Component Analysis (PCA) plot of murine pluripotent cell samples (red) including samples produced via electroporation. Samples with similar gene expression profiles appear closer to one another in the three-dimensional space. represented by the plot. The control (blue) sample represents murine hepatic cells. Clustering with and among natively pluripotent (murine ES) cells and cells induced to pluripotency genetically, were murine cell samples induced to pluripotency (black circle) by electroporation (300V, 70 pulses, 5 ms, pulse interval 100 ms) alone or in the presence of Oct4, Sox2, Nanog proteins (50 ug per protein) or by overexpression of the long PRR+ Numb isoform. Electroporation of ˜200,000 mouse fibroblast cells in 200 ul of PBS was performed in phosphate buffered saline (PBS) at 4C in a 4 mm gap cuvette using a BTX ECM 830 electroporation machine. Electroporated cells were maintained in growth medium (GM) containing Dulbecco's Modified Eagle Medium (DMEM) supplemented with 10% fetal bovine serum (FBS), 20 mM L-glutamine, and 1% penicillin/streptomycin. After electroporation, GM was supplemented with 4 ng/ml bFGF and the medium changed every other day. Global gene expression was assayed in all of the samples using the Affymetrix GeneChip HTA 2.0 chip.

FIG. 2 shows a Heatmap corresponding to the samples displayed in the PCA plot of FIG. 1, and wherein the sample of cells induced to pluripotency by electroporation alone (black arrow) are located within larger clusters representing murine IPS cells and ES cells-evidence that the treated cells are indistinguishable by global gene expression from published pluripotent cell populations.

FIG. 3A depicts a low power brightfield photo of treated cells 24 hours after electroporation alone (in the absence of reprogramming factors).

FIG. 3B shows a corresponding fluorescent image of cells expressing eGFP under the control of a c-MYC promoter (a pluripotency reporter construct).

FIG. 4A depicts treated cells at low power 72 hours after induction with electroporation alone with subsequent incubation in medium containing DMEM supplemented with bFGF.

FIG. 4B depicts treated cells 72 hours after induction of pluripotency with electroporation in the presence of Oct4, Sox2, and Nanog proteins.

FIG. 4C shows a fluorescent image at low power corresponding to the brightfield photo in FIG. 4A and reveals cells (red boxes) expressing eGFP under the control of a c-MYC promoter (a pluripotency reporter construct).

FIG. 4D shows a fluorescent low power image corresponding to the brightfield photo in FIG. 4B and shows an even greater number of cells (red boxes) expressing eGFP under the control of a c-MYC promoter (a commercially-available pluripotency reporter construct).

FIG. 4E. shows the same fluorescent image as the one depicted in 4C, but without the boxes.

FIG. 4F. shows the same fluorescent image as the one depicted in 4D, but without the boxes.

FIG. 5A depicts murine fibroblast cells at low power 72 hours after induction with electroporation in the presence of Oct4, Sox2 and Nanog proteins (50 ug per protein; 300V, 70 pulses, 5 ms, pulse interval 100 ms).

FIG. 5B shows a low power fluorescent image corresponding to the brightfield photo of FIG. 5A and reveals that ˜100% of cells electroporated in the presence of Oct4, Sox2 and Nanog proteins are induced to express eGFP under the control of the c-MYC promoter.

FIG. 5C depicts treated cells 6 days after exposure to factor free Electroporation. Numerous small colonies of rounded cells are apparent. although some cells retained flattened or stellate appearance of fibroblast cells.

FIG. 5D shows treated cells 6 days after exposure to Electroporation with Oct4, Sox2 and Nanog proteins. Small colonies of rounded cells are present throughout. By 6 days, no flattened or stellate cells were observed in this arm of the experiment.

FIG. 5E depicts a very large colony of what appear to be hundreds of very small cells, consistent with an embryoid body, 6 days after factor free Electroporation.

FIG. 5F shows a higher magnification of the embryoid body depicted in FIG. 5E.

FIG. 6A shows small cell colonies and early embryoid formation in cells treated 12 days earlier with factor free Electroporation.

FIG. 6B depicts individual cells, small colonies and enhanced embryoid body formation 12 days after exposure to Electroporation in the presence of Oct4, Sox2 and Nanog proteins.

FIG. 7A shows a brightfield photo of embryoid 23 days after induction of murine fibroblast cells with Electroporation in the presence of Oct4, Sox2 and Nanog.

FIG. 7B shows a highly magnified view of embryoid body and embryoid bodies 23 days after induction of murine fibroblast cells with Electroporation in the presence of Oct4, Sox2 and Nanog.

FIG. 7C-7F depict immunohistochemistry in embryoid bodies thirty days after Oct4/Sox2/Nanog Protein Electroporation of mouse fibroblast cells. The embryoid showed positive reactivity with anti-Oct4 (FIG. 7C), anti-Nanog (FIG. 7D), anti-Numb (FIG. 7E) and anti-Notch antibodies (FIG. 7F).

FIGS. 7G-7I illustrate Oct4, Sox2 and Nanog treated cells growing and persisting as large embryoid bodies on day 40 (FIG. 7G and FIG. 7H), and large rafts of embryoid by day 57 (FIG. 7I).

FIG. 8A depicts a 1-8 cell stage embryo showing the cells induced to induced potency with Electroporation are competent to generate an embryo, consistent with totipotency.

FIG. 8B depicts 3 blastocyst stage embryos showing the cells induced to induced potency with Electroporation are competent to generate a complete embryo, definitional of totipotency.

FIG. 8C-8H depict Embryos induced from mouse fibroblast cells showing various morphologies consistent with peri-implantation stages of embryonic development. Morula (8C) and early egg cylinder (8D) are depicted in high power (40×) micrographs along with paired, phase contrast and HDR brightfield images of late stage blastocysts (8E-8H) and hatched blastocyst (8I and 8J).

FIGS. 9A-9H depicts treated cells at low power magnification 48 hours after exposure to electroporation at 300V with varying number (20-200) 5 ms pulses in the presence of FITC-conjugated albumin along with corresponding frequency histograms showing progressively increased, fluorescence intensity. FIG. 9I depicts similar appearing cells after analogous treatment using a magnetic field generator in the presence of ethidium bromide Novickij et al., 2013). FIG. 9J depicts the % of cells positive for EtBr with 200 or 300 pulse condition.

FIG. 9K shows plots of minimum (blue), modal (red), and maximum (green) fluorescence intensity observed for each pulse condition depicted in Panels B, D, F, and H.

FIGS. 10A-10 D illustrate cell culture dishes with reservoirs for electroporation.

FIG. 10E illustrates an assembly of sample cell culture dishes (number of dishes may vary for example from 1 to 1,600) wherein each has a “reservoir” portion that functions like a typical electroporation cuvette and broader portion (“inspection plane”) which functions like a traditional cell culture plate.

FIG. 11A illustrates a catheter style electroporation/magnetoporation apparatus suitable for in vivo electroporation with protein and other transfectants.

FIGS. 11B, 11C illustrate distal ends of catheters suitable for in vivo application of an Electric field, including retractable needle with co-axial electrodes and circular electrode array circumscribing a retractable injection needle.

FIG. 11D illustrates a loop or circular electrode array which is used in the catheter (shown in FIG. 11C suitable for in vivo electroporation.

FIG. 12A is a schematized vector map corresponding to the vector sequence of Example 13. FIG. 12B shows a non-integrating, Numb and Numblike lentivectors.

FIGS. 13A and 13B illustrate cells transfected with PRR+Numb, which form colonies of very small cells that express pluripotent marker SSEA3 as assessed by fluorescent immunohistochemistry.

FIGS. 13C and 13D illustrate cells transfected with PRR+Numb which form colonies of very small cells that express pluripotent marker SSEA4 as assessed by fluorescent immunohistochemistry.

FIGS. 14A and 14B illustrate that following electroporation with Oct4, Sox2 and Nanog proteins, treated cells form colonies of very small cells that express pluripotent marker SSEA4 as assessed by fluorescent immunohistochemistry.

FIGS. 14C and 14D illustrate that following electroporation with Oct4, Sox2 and Nanog proteins, treated cells form colonies of very small cells that express pluripotent marker SSEA3 as assessed by fluorescent immunohistochemistry.

FIGS. 15A and 15B illustrate human buccal cheek cells electroporated at 300V in the presence of FITC-conjugated albumin and captured with brightfield (FIG. 15A) and fluorescent (FIG. 15B) microscopy 48 hours after electroporation. In preparation for electroporation, Buccal cheek cells were collected from cell culture and resuspended at 1×106 cells/ml in PBS. Electroporation was carried out using the BTX ECM 830 electroporation machine according to the methods of Koken et al., 1994. Briefly, —200,000 cells were transferred to 4 mm cuvettes and electroporated at 300 V in the presence of either 150 ug of FITC-conjugated albumin or 150 ug of oct4, sox2, and nanog protein cocktail (pulse length=5 m, pulse interval=100 ms). Immediately following electroporation, the cells were transferred to Dulbecco's Modified Eagle Medium (DMEM) supplemented with bFGF in standard cell culture plates, and incubated at 37 degrees Celsius.

FIGS. 16A-16C illustrate cell colonies induced from buccal cheek cells at 6 days (FIG. 16A and FIG. 16B) and 46 days (FIG. 16C) after electroporation.

FIGS. 17A-17C illustrate that 7 weeks after electroporation cell colonies induced from buccal cheek cells using electroporation express pluripotency associated genes Oct 4 (FIG. 17A), Nanog (FIG. 17B), and Sox2 (FIG. 17C).

FIGS. 18A-18I illustrate that protein electroporation with Oct4, Sox2 and Nanog produces developmentally-activated, buccal cheek cells that show embryoid formation (rectangles) and positive reactivity for Nanog (FIGS. 18A-18C), Sox2 (FIGS. 11D-11F) and Oct4 (FIGS. 11G-18I).

FIGS. 19A-19C illustrate that protein electroporation with Oct4, Sox2 and Nanog produces developmentally-activated, buccal cheek cells that show embryoid formation composed of very small cells that show positive reactivity for Nanog (FIG. 19A), Sox2 (FIG. 19B) and Oct4 (FIG. 19C).

FIG. 20A illustrates successful construction of the pLenti6-MSGW/EmGFP-Bsd/EF1a/miR-decoy vector confirmed by Restriction Digestion.

FIGS. 20B-20C illustrate successful transfection with the pLenti6-MSGW/EmGFP-Bsd/EF1a/miR-decoy vector confirmed by visualizing syncitia formation at 72 hours (FIG. 20B) versus control (FIG. 20C) wherein virus stock was prepared from transfected 293FT cells.

FIG. 20D is a pLenti6-MSGW/EmGFP-Bsd/EF1a/miR-decoy vector map.

FIGS. 21A-21F illustrate that combination of long PRR+Numb transfection with Notch and/or Oct4/Sox2/Nanog protein transfection (per Koken et al., 1994; 300V, 70 pulses, 5 ms pulse length) produces embryoid formation and pluripotency.

FIG. 22 illustrates embryoid bodies 35 days post electroporation.

FIGS. 23A-23B graphically illustrate the Numb and Numblike protein structure (23A) and related HIV-EGFP-Numblike, HIV-EGFP-Numb and control, non-integrating lentivectors (23B).

FIGS. 24A-24F illustrate that in vivo injection of the HIV-EGFP-Numblike transfectant into the lateral ventricle and subsequent electroporation caused embryonic (E)15 cells to exit the germinal zone and express neuronal Hu C/D as they migrated radially.

FIGS. 25A-25F illustrate that following in vivo injection of the HIV-EGFP-Numblike transfectant into the lateral ventricle and subsequent electroporation, cortical ventricular zone cells migrated radially, expressed beta-tubulin, and differentiated into neurons.

FIGS. 26A-26C illustrate that electroporation of mouse ventricular zone cells at P0 with HIV-EGFP Numblike and HIV-EGFP control vector produces cells with divergent cell morphologies, migrating neurons and radial glia respectively, —evidence that numblike acts as a cell fate determinant.

FIGS. 27A and 27B illustrate a ventricular zone cells electroporated at P0 with HIV-EGFP control vector at low (27A) and high magnification (27B).

FIGS. 28A, 28B illustrate that intraventricular injection of the HIV-EGFP-Numblike transfectant followed by in vivo electroporation upregulates Numb expression.

FIGS. 29A-29C illustrate that in vivo injection of the HIV-EGFP-Numb PTB−/PRR− transfectant followed by electroporation promotes migration and neuronal differentiation in postnatal mice.

FIGS. 30A-30F illustrate that transiently expressed EGFP does not correlate with GLAST protein, a marker of radial glia, but strongly correlates with Numb, TUJ, and DCX markers of neuronal differentiation following HIV-EGFP-Numblike injection and electroporation, in vivo.

FIGS. 27A-27F illustrate that HIV-EGFP-Numb PRR−/PTB− (FIGS. 27B and 27E) and HIV-EGFP-Numblike (FIGS. 27C and 27F) lentiviruses reduce proliferation and promote differentiation in Ras+, Breast cancer cells versus control (FIGS. 27A and 27D) at 5 days (FIGS. 27A-27C) and ten days (FIGS. 27D-27E) post-transduction.

### DETAILED DESCRIPTION

The present invention may be used with any suitable cells, including vertebrate cells and invertebrate cells, including fish, mammals, birds, amphibians, and reptiles and insects cells.

A theoretical basis for the embodiments of the invention is described herein, however, this discussion is not in any way to be considered as binding or limiting on the present invention. Those of skill in the art will understand that the various embodiments of the invention may be practiced regardless of the model used to describe the theoretical underpinnings of the invention.

The cited references, patents, and patent applications herein, to the extent that they provide exemplary procedural or other details supplementary to those set forth herein, are specifically incorporated herein by reference.

Likewise, all patents, patent applications, references and publications cited in this application are hereby incorporated by reference herein in their entireties.

The practice of the techniques described herein may employ, unless otherwise indicated, conventional techniques and descriptions molecular biology (including recombinant techniques), cell biology, biochemistry, and genetic engineering technology, which are within the skill of those who practice in the art. Such conventional techniques and descriptions can be found in standard laboratory manuals such as Green and Sambrook, Molecular Cloning: A Laboratory Manual. 4th, ed., Cold Spring Harbor Laboratory Press, Cold Spring Harbor, N.Y., (2014); Current Protocols in Molecular Biology, Ausubel, et al. eds., (2017); Neumann, et al., Electroporation and Electrofusion in Cell Biology, Plenum Press, New York, 1989; and Chang, et al., Guide to Electroporation and Electrofusion, Academic Press, California (1992), all of which are herein incorporated in their entirety by reference for all purposes.

### Definitions

As discussed herein, “DNA” refers to deoxyribonucleic acid and “RNA” refers to ribonucleic acid. As discussed herein, “cDNA” refers to complementary DNA; “mRNA” refers to messenger RNA; “siRNA” refers to small interfering RNA; “shRNA” refers to small hairpin RNA; “miRNA” refers to microRNA, such as single-stranded RNA molecules, typically about 20-30 nucleotides in length, which may regulate gene expression; “decoy” and “decoy RNA” and “RNA decoy” refer to an RNA molecule that mimics the natural binding domain for a ligand.

As used herein, the meaning of the term “ameliorating” includes lessening an effect, or reducing damage, or minimizing the effect or impact of an action, activity, or function, and includes, for example, lessening the deleterious effects of a disease or condition.

As used herein, the meaning of the term “retarding” includes slowing or lessening the progress of an effect or action, and includes, for example, slowing the progress of a disease, slowing the rate of infection, or otherwise acting to slow or reduce the advance or progress of a disease or condition.

As used herein, an “inducing agent” is an agent that aids or is alone effective to promote an action. For example, an exogenous agent that affects a promoter, e.g., by initiating or enhancing its activity, and so affects expression of a gene under control of the promoter, may be termed an inducing agent. For example, tetracycline may be used as an inducing agent; and doxycycline may be used as an inducing agent.

A nucleic acid sequence (e.g., a nucleic acid sequence encoding a polypeptide) is termed “operably linked” to another nucleic acid sequence (e.g., a promoter) when the first nucleic acid sequence is placed in a functional relationship with the second nucleic acid sequence. For example, a promoter is operably linked to a coding sequence if the promoter affects the transcription or expression of the coding sequence. As used herein, the term “driven by” refers to a gene or coding sequence that is operably linked to a promoter sequence, and that the promoter sequence affects the transcription or expression of the coding sequence.

As used herein, a “marker” is a molecule that is detectable, or codes for a detectable molecule, or acts on other molecules so that the presence of the marker is detectable. A “marker protein” or “marker polypeptide” is a protein or polypeptide that is detectable in a laboratory or clinical environment, and, in embodiments, may be detectable by eye. A “marker gene” encodes a marker protein or marker polypeptide.

As used herein, “HIV” refers to human immunodeficiency virus, and includes variants such as, e.g., HIV-1, HIV-2. Other immunodeficiency viruses include simian immunodeficiency virus (SIV) and feline immunodeficiency virus (FIV). HIV Proteins may be termed immunodeficiency virus “enzymes” or “accessory proteins” and include, for example, integrase, protease, reverse transcriptase, and transactivating regulatory protein (TAT), etc.

Developmentally-active cells (DAC), as defined herein, represent a broad category of cells that are either i. “transitory-type cells” typically showing high potency, such as embryonic stem (ES) cells, very small embryonic-like (VSEL) cells, totipotent cell stem cells, pluripotent stem cells, multipotent stem/progenitor cells), or ii. “fixed-type” cell types which typically have low or limited potency, (e.g. differentiating somatic cell types capable of further differentiation and/or integration in vivo-usually over the course of an organism's development).

While “transitory” DAC tend, in nature, to divide and/or show self-renewal, “fixed” DAC are usually post-mitotic and usually will not show self-renewal. Instead, fixed, DAC typically show the emergence of some characteristics associated with a terminally-differentiating cell of a specific type. Nevertheless, both “transitory” and “fixed” cells have uses and potential uses in medicine (especially regenerative medicine, transplantation medicine, veterinary medicine, animal husbandry, drug-discovery, drug-testing, gene therapy, tissue-engineering, organ production, and biological modeling), laboratory-based food production, and various other industries.

As a category, developmentally-active cells (DAC) may include cells that inherently show features of developmentally active cells, as well as cells that have been “activated”, aka “made”, “forced”, “induced, or “reprogrammed” to acquire such characteristics; this latter subset of developmentally active cells being defined herein as “developmentally-activated cells (DAdC)”.

Writing with respect to with respect to B cells and T cells of the immune system, Kolanus et al., (1992) observed that a “developmentally activated cell state” results from “a change in transcriptional potential”. Likewise, as used herein, “developmentally-activated cells” (DAdC) comprise cells which some skilled in the art term, “reprogrammed cells”, “partially-reprogrammed cells”, “induced pluripotent cells”, “directly reprogrammed cells”, “indirectly-reprogrammed cells”, “pluripotent-like cells”, etc., (e.g. induced totipotent cells, induced pluripotent cells, induced multipotent cells, induced hematopoietic stem/progenitor cells, induced neurons, induced cardiac cells, induced skeletal muscle cells, induced cartilage cells, induced hematopoietic cells, induced liver cells, induced pancreatic beta cells, etc.)—as such cells show a change in transcriptional potential relative to untreated cells.

As used herein, terms of art, such as “totipotent”, “pluripotent”, “multipotent”, “self-renewing”, “differentiating”, “cardiac”, “muscle”, “neuron”, “progenitor”, “stem cell”, “osteoblast”, “chondrocyte”, etc. are understood to refer either to i. cells produced in nature (natural cells) of a type, or to ii. like cells (similar cells) produced through the methods described herein-such like cells displaying some, but not necessarily all, features marking the natural cells denoted by these terms. For example, as used herein, a “totipotent cell” is one competent to produce a complete embryo, but need not meet any other criteria familiar to skilled persons. As used herein, “pluripotent cell” is one capable of forming embryoid in vitro and/or one that expresses markers of pluripotency or a transgene (e.g. EGFP) associated with a pluripotency reporter construct, however such a cell need not form teratomas in vivo or show other features of pluripotency familiar to those skilled in the art; accordingly, as used herein, “pluripotent cells” need not meet all of the several criteria for pluripotency commonly applied by some skilled in the art. Conversely, some cells sometimes considered as “pluripotent” by those skilled in the art are considered herein to represent inherently, developmentally-active cell types, e.g. embryonic stem cells (ES) cells, Very Small Embryonic-Like (VSEL) stem cells, mouse embryonic stem cells (mES), etc. In regard to such cells, Kim et al. (2014), write,
“Pluripotent stem cells (PSCs) have been considered as the most important cells in regenerative medicine as they are able to differentiate into all types of cells in the human body. PSCs have been established from several sources of embryo tissue or by reprogramming of terminally differentiated adult tissue by transduction of so-called Yamanaka factors (Oct4, Sox2, Klf4, and cMyc). Interestingly, accumulating evidence has demonstrated the residence of PSCs in adult tissue and with the ability to differentiate into multiple types of tissue-committed stem cells (TCSCs). The applicants also recently demonstrated that a population of pluripotent Oct4(+), SSEA-1(+), Sca-1(+), Lin(−), CD45(−) very small embryonic-like stem cells (VSELs) resides in the adult murine bone marrow (BM) and in other murine tissue. These very small (˜3-6 μm) cells express pluripotent markers such as Oct4, Nanog, and SSEA-1. VSELs could be specified into several tissue-residing TCSCs in response to tissue/organ injury, and thus suggesting that these cells have a physiological role in the rejuvenation of a pool of TCSCs under steady-state conditions. In this review article, the applicants discuss the molecular nature of the rare population of VSELs which have a crucial role in regulating the pluripotency, proliferation, differentiation, and aging of these cells (Kim Y, Jeong J, Kang H, Lim J, Heo J, Ratajczak J, Ratajczak M Z, Shin D M. The molecular nature of very small embryonic-like stem cells in adult tissues. Int J Stem Cells. 2014 November; 7(2):55-62; https://www.ncbi.nlm.nih.gov/pubmed/25473442).

It follows that the pluripotent, VSEL cells and tissue-committed stem cells TCSC's of Kim et al., represent cell types included in the category of developmentally active cells (DAC) as defined herein.

### Embryoid and Embryoid Bodies

Lin and Chen (2014) indicate, “Embryoid bodies (EB) are the three-dimensional aggregates formed in suspension by pluripotent stem cells (PSC), including embryonic stem cells (ESC) and induced pluripotent stem cells (iPSC).”

Pettinato et al. (2015) further describe embryoid bodies as,
“three-dimensional (3D) hPSC [human pluripotent stem cell] aggregates that can differentiate into cells of all three germ layers (endoderm, ectoderm, and mesoderm) [3]. Many events in the in vitro lineage-specific differentiation process within the EBs recapitulate those seen in vivo in the developing embryo [6], which justifies the uses of EBs as a model system to simulate the in vivo differentiation of hPSCs under in vitro culture conditions, and mechanistically examine hPSC differentiation programs/lineage commitment during embryogenesis as an alternative to the whole embryo approach [7]. In addition, in vitro formed EBs have opened access to early precursor cell populations that are not accessible in vivo [8]. EBs have been shown to effectively initiate lineage-specific differentiation of hPSCs toward many lineages, such as cardiac [9], neural [10,11], hematopoietic [12], and pancreatic β cells [13].”

In part, the current invention is directed to the production of “transitory type” developmentally-activated cells (DAdC) that display many features associated with totipotent, pluripotent, multipotent, and/or self-renewing cells, e.g. the capacity to form embryoid (aka embryoid bodies) when cultured in vitro. Thus, Embryoid formation is a behavior those skilled in the art often associate exclusively with pluripotent cells.

As used herein, formation of embryoid bodies by cells “developmentally-activated” according to methods described herein indicates that these newly, developmentally-activated cells (DAdC) are fully pluripotent, as the term is used herein.

Teratomas contain cells from the three germ layers: ectoderm, mesoderm, and endoderm. Teratoma formation is, however, also associated with carcinogenicity. Some skilled in the art may consider that a cell cannot be termed a “pluripotent” cell unless it forms a teratoma when injected in vivo, even if this tendency to tumorigenesis is considered highly undesirable in cells intended for clinical use, especially in regenerative medicine,

The developmentally-activated cells produced according to the methods taught herein need not form teratomas in order to meet the criteria of “pluripotent” as used herein. AS used herein, such cells may nevertheless represent “transitory-type, developmentally activated cells (DAdC)”. In contrast, the transitory DAdC produced according to the methods described herein, show one or more desirable characteristics associated with pluripotency, such as small size, expression of pluripotent markers such as SSEA3/SSEA4, Oct4, Nanog, Sox2, as well as colony formation and embryoid formation, and therefore such transitory developmentally activated cells (DAdC) are termed “pluripotent” and/or “pluripotent-like”, herein.

Similarly, the current invention is directed in part, to the production of “differentiating cells” (aka differentiating somatic cells, aka somatic differentiating cells) that display some, but not necessarily all, cell type specific markers associated with the desired cell type. Those skilled in the art may differ somewhat as to the criteria for defining a pluripotent cell as well as differentiating and/or differentiated cell types. As there exists no universally accepted terminology for cells induced or activated to differentiating somatic cell types, the applicants refer to various desired, differentiating cell types producible by the methods described herein as cardiac cells, neurons, chondrocytes, cartilage cells, bone cells, hepatocytes, etc.; however these terms, when used herein also refer to “cardiac-like cells”, “neuron-like cells”, “chondrocyte-like cells”, “cartilage-like cells”, “bone-like cells”, “hepatocyte-like cells”, etc.

Thus, the criteria for defining a desired cell type or degree of potency, as taught herein, may not coincide precisely or overlap entirely with the various criteria taught by others skilled in the art for defining a cell type or a cell potency, and the invention is therefore not bound by such various definitions. Instead, the current invention is aimed at producing cells displaying certain desirable features, characteristics and behaviors which commend them for the various and specific uses taught herein.

The induced pluripotent stem cells of Takahashi and Yamanaka, which were generated, in part, using oncogenes c-myc and klf4, were able to form teratomas when injected in vivo. Likewise, when Takahashi and Yamanaka produced embryos from these induced pluripotent stem cells, a large percentage of the resulting animals developed tumors postnatally (Takahashi and Yamanaka, 2006; Takahashi et al., 2007). Thus, teratoma formation and carcinogenicity are features associated with some such induced pluripotent cells, and are features that some skilled in the art may consider undesirable with respect to providing sources of replacement cells, cells for transplantation, cells for gene therapy, cells for tissue engineering, and cells for various other clinical uses.

In contrast, the transitory, pluripotent and pluripotent-like, developmentally-activated cells (DAdC) produced according to the methods taught herein tend to 1. display colony formation, 2. form embryoid, 3. Express various genes and proteins associated with pluripotency, 4. cluster with ES and iPS cells in hierarchical cluster analysis based on their gene expression (e.g. global gene expression profile), and 5. cluster with ES and iPS cells in Principal Component Analysis (PCA) plots based on their gene expression (e.g. global gene expression profile), but 6. may fail to demonstrate the teratoma formation and the carcinogenicity frequently associated with ES cell lines and induced pluripotent stem cells.

Developmentally activated cells (DAdC), may be recognized and defined, for example, by i. gene expression analysis (e.g. by reactome overrepresentation analysis using hypergeometric distribution) that reveals overrepresentation (or enrichment) of genes associated with certain cellular pathways, especially the Cell Cycle pathways and the Developmental Biology pathways, in REACTOME analyses for “fixed” type, developmentally-activated cells (DAdC).

Such enriched gene expression (as demonstrated by Reactome analysis) is consistent with the adaptability of DAdCs as a source of stem-like cells, replacement cells, cells suitable for gene therapy and tissue engineering, and cells differentiable to various types (according to the methods disclosed herein, for example, multipotent and pluripotent cells.

The developmentally-activated cells (DAdC) of the present invention also typically display one or more of microscopically-visible, induced changes in cell behavior related to ii. colony formation, iii. embryoid formation and iv. cell size. Likewise, v. immunohistochemistry may reveal the “transitory” developmentally activated cells as expressing multiple markers commonly associated with pluripotency, such as such as Oct4, Nanog, c-Myc, Notch, SSEA3/4; and TRA-1-81; while “fixed” type developmentally-activated cells will show some markers of differentiating or terminally differentiated cells.

In the present invention the applicants teach that the application of Energy in the form of an electrical field or charge (z) as a means of developmentally activating adult or somatic cells.

In accordance with the present invention, various means of detecting, monitoring, and demonstrating induced cellular phenotype include gene reporter assays (e.g. performed with a reporter construct) wherein a reporter gene's expression (e.g. an antibiotic resistance gene or fluorescent reporter gene) is linked to the promoter of a gene upregulated in DACs (e.g. c-Myc, Nanog, Oct4, DCX, etc.), as well as other assays described herein.

However, as the term is used herein, Developmentally-activated cells or DAdC need not satisfy all criteria of pluripotency to be recognized as DAdC in the current invention, and therefore may not be pluripotent in the opinions of some skilled in the art, even if they do display desirable characteristics such as very small size, embryoid formation in vitro, and pluripotency marker expression, and other features that some skilled in the art consider tantamount to pluripotency; therefore to the extent they are similar to and display features overlapping with cells which have been commonly or unambiguously recognized as pluripotent, some DAdC may be considered herein and by some skilled in the art to be “pluripotent-like”, “ES-like” or even “VSEL-like”.

Accordingly, and to avoid confusion, the term, developmentally-activated cell (DAdC) is applied herein and is defined by many features these cells display morphologically, microscopically, and immuno-histologically, as well as according to their pattern of gene expression (transcriptome), their reactome, their utility, and many of their potential uses.

It should be understood that a developmentally-activated cell (DAdC) may one caused to display some features consistent with either “transitory” (pluripotent-like, multipotent-like, and/or self renewing cells) or “fixed” (somatic-like, differentiated-like or differentiating-like) cellular phenotypes, as described herein, and that the present invention enables the production and interconversion of these cells by efficient introduction or overexpression of nucleic acids or proteins corresponding to specific transcription factors, cell fate determinants, small RNAs, and/or aptamers, or by chemical and/or physical means, either in vivo or in vitro, in the presence or absence of specialized cell culture conditions.

By the same token, while terms for somatic, differentiated cells such as “cardiac cell”, “neuron”, “liver”, “chondrocyte”, “osteoblast”, “T cell”, “beta cell”, etc. appear herein as desirable cells producible by the methods described herein, such terms are used, herein, to describe “developmentally-activated cells” (DAdC) that are similar to corresponding cells (expressing certain cell type specific markers), i.e. cardiac-like cells, neuron-like cells, liver-like cells, chondrocyte-like cells, osteoblast-like cells, T-like cells, beta-like cells, etc. Accordingly, developmentally-activated cells (DAdC) which are of the “fixed type” i. display some but usually not all markers of the desired, cell type (as assessed by Reactome, transcriptome, gene expression and/or protein expression assays), ii. are capable of survival, further differentiation and/or integration in vivo, and iii. need not meet all criteria that those skilled in the art may sometimes apply to the mature phenotype that the DAdC approximate.

The present invention relates to equipment and apparatuses suitable for use in applying an Electrical field to a cell or tissue, especially application producing developmental activation in a cell, and especially application producing developmental activation in a cell.

The current invention teaches related equipment for electroporating or magnetoporating cells ex vivo, in vitro, or in a tissue or organ in vivo.

Farley et al., (2011) have taught that the application of an electrical field to a cell via electroporation has little to no effect on gene expression, writing, “Both current alone and in combination with exogenous DNA expression have a small but reproducible effect on endogenous gene expression, changing the expression of the genes represented on the array by less than 0.1% (current) and less than 0.5% (current+DNA), respectively.” Farley et al., further observed, “This indicates that . . . the addition of DNA does alter the tissue's response to the current.” Farley et al., further state, “these findings represent the first systematic genome-wide analysis of the effects of in ovo electroporation on gene expression during embryonic development. The analysis reveals that this process has minimal impact on the genetic basis of cell fate specification.”

The present invention, therefore, teaches away from Farley et al., (2011) in that it teaches that electroporation alone, or in the presence of nucleic acids or proteins, has the ability to effect significant changes in global gene expression as well as to significantly impact cell fate specification. Specifically, the present invention teaches that electroporation alone, or in conjunction with transcription factors and/or other cell fate determinants, can induce cells to a wide variety of cell types, including pluripotent or totipotent, ES-like cells that can differentiate to multiple lineages and cell types.

In one part, the present invention teaches a catheter capable of infusing various agents, e.g. therapeutic or diagnostic agents, into a tissue or an organ, wherein the catheter comprises an injection needle that, in some embodiments, may also serve as an electrode for electroporation, including for protein electroporation. Likewise, a catheter suitable for megnetoporation will feature a coil in place of an electrode pair.

The electroporation or magnetoporation catheter of the present invention is, in some embodiments, characterized by extendable and retractable needle suitable for application of an electrical field to a cell or tissue in vivo, with or without injection of an agent, transfectant, or cargo into the target tissue, organ or cavity tissue.

In some embodiments, the electroporation/magnetoporation catheter further comprises a flexible tubing having proximal and distal ends and at least one lumen.

The needle of the present invention may be of any suitable medical grade construction. The needle, which in some embodiment may double as electrode, will be electrically isolated from other electrodes and their lead wires.

In some embodiments, the retractable needle extends beyond the distal end of electrode catheter tubing during injection.

In some embodiments, the needle is connected to a water-tight tube which is connected proximally to an injection controller.

In some embodiments, the injection controller is a syringe.

In some embodiments, the injection controller is a bladder.

In some embodiments, the injection controller is a mechanical pump allowing finely-controlled and/or programmed control of the injection rate.

In some embodiments, a radiologically detectable tracer or marker is injected via the needle to facilitate monitoring of successful injection and/or electroporation. For instance, said marker may be any known to the art that is detectable by X-ray, MRI, or nuclear imaging methods.

The needle may be of any size deemed suitable for practice of the invention by the medical interventionalist skilled in the art.

In some embodiments, the needle features multiple opening through which a marker, transfectant, cargo or other agent may be injected.

In some embodiments, wherein the needle may double as an electrode, it is further connected to a lead wire. In some embodiments, the injection needle may function as an electroporation electrode in combination with a second electrode(s). In some embodiments, the injection needle may function as an electrode in combination with multiple coaxially located electrodes. In some embodiments, the injection needle may function solely as an electrode or as an electroporation electrode in combination with a loop electrode. In some embodiments, the injection needle may function as an electroporation electrode in combination with a circular array of electrodes.

In some embodiments, the injection needle may function as an electroporation electrode in combination with a circular array of needle electrodes. In some embodiments, the injection needle does not double/function as an electroporation electrode, and, instead, coaxially located electrodes perform the electrode function. In some embodiments, the injection needle does not double/function as an electroporation electrode, and, instead, a loop electrode, alone or in combination with coaxially located electrodes, performs the electrode function. In some embodiments, the injection needle does not double/function as an electroporation electrode, and, instead, the electrodes of a circular array perform the electrode function. In some embodiments, the electrodes of the electroporation/magnetoporation catheter are retractable electrodes. In some embodiments, the retractable electrodes may be extended beyond the distal end of catheter during electroporation. In some embodiments, the electrodes are extended to prior to injection to ascertain the catheter is correctly positioned, and may be used to detect a change in impedance when the catheter elements are in contact with tissue.

In some embodiments, the electrodes and/or needle doubling as an electrode may be used detect and/or map electrical activity in a tissue prior to injection of an agent or transfectant and prior to electroporation. In some embodiments, the electrodes and/or needle doubling as an electrode may be used detect and/or map electrical activity in a tissue prior to injection of an agent or transfectant and prior to electroporation. In preferred embodiments, the electrodes and/or needle doubling as an electrode are connected electrically to an electrical pulse generator and power supply. In some embodiments, the entire catheter system of the present invention is connected to a computer interface and/or computer algorithms by which the system is monitored and controlled. In some embodiments, the electrodes and/or needle doubling as an electrode may be used to perform irreversible electroporation. In some embodiments, a needle hand control is provided at the proximal end of the catheter tubing. In some embodiments, injection needle system extends from the distal end section, through the catheter tubing to a needle controller.

In some embodiments, the injection needle can translocate so that its distal end can extend under the influence of the needle hand control. In some embodiments, electrodes are mounted on the distal end of the catheter as coaxially positioned electrodes, loop electrode(s) and/or circular electrodes. In some embodiments, multiple electrodes are mounted on the distal end of the catheter as a circular electrode array. In some embodiments, an electrode lead wire is electrically connected to the injection needle and to a suitable monitoring apparatus or to a power source. In some embodiments, the invention is directed to a method for introducing an agent, especially a therapeutic, drug, transfectant, or a cellular cargo. In some embodiments, the invention is directed to a method for developmentally activating a cell or cell population in the tissue of a patient. In some embodiments, the method comprises introducing the distal end of a catheter into or through the patient's body, vasculature, or through an orifice to reach the target tissue or space, wherein the injection needle is then extended beyond the distal end of the end section and an electrical field is applied to the target cell(s).

In some embodiments, the method comprises introducing the distal end of a catheter into or through the patient's body, vasculature, or through an orifice to reach the target tissue or space, wherein the injection needle is then extended beyond the distal end of the end section and a useful agent, especially a marker, therapeutic, drug, transfectant, agent, and/or cell population, is then injected into the tissue, organ, space, cavity, etc.

In some embodiments, the method comprises introducing the distal end of a catheter into or through the patient's body, vasculature, or orifice to reach the target tissue or space, wherein the electrodes and/or needle doubling as an electrode are used to perform reversible electroporation. In some embodiments, the method comprises introducing the distal end of a catheter into or through the patient's body, vasculature, or orifice to reach the target tissue or space, wherein the electrodes and/or needle doubling as an electrode are used to perform irreversible magnetoporation/electroporation. In some embodiments, the tissue of the present invention, the target tissue is one that is injured, genetically compromised, metabolically compromised, ischemic, that has suffered ischemic damage, or that harbors cancerous cells. In some embodiments, the tissue of the present invention is a tissue that has suffered ischemic damage and the agent or transfectant or cell population injected is capable of mitigating said damage. In some embodiments, the tissue of the present invention is a tissue that is genetically-compromised or metabolically-compromised and the agent or transfectant is capable of mitigating said compromise. In some embodiments, the tissue of the present invention is a tissue in need of regeneration. In some embodiments, the tissue of the present invention is a tissue in need of regeneration and the agent or transfectant is capable of promoting said regeneration. In some embodiments, the tissue of the present invention is a tissue in need of repair. In some embodiments, the tissue of the present invention is a tissue in need of repair and the agent or transfectant is capable of promoting said repair. In some embodiments, the tissue of the present invention is a tissue in need of differentiated cellular elements and the agent or transfectant is capable of promoting said differentiation. In some embodiments, the tissue of the present invention is a tissue featuring abnormal growth and/or proliferation (e.g. cancer) and the agent or transfectant is capable of reducing or elimination said abnormal growth and/or proliferation. In some embodiments, the tissue of the present invention is a tissue featuring hypoplasticity, and the agent or transfectant is capable of promoting growth and/or proliferation. In some embodiments, the agent or transfectant is a protein or nucleic acid and/or transfection mediating nanoparticle and/or transfection mediating reagent, named herein. In some embodiments, the agent or transfectant is an element, chemical compound, drug or cellular extract. In some embodiments, a tissue is injected with an agent, transfectant or a cell population, but electroporation is not applied. In some embodiments, in vivo, irreversible electroporation is performed after injection of an agent or transfectant.

In some embodiment, the catheter of the present invention comprises an catheter tubing having proximal and distal ends, an end section at the distal end of the catheter tubing, and a needle hand control and deflection controller proximal the catheter tubing. In some embodiments, the tubing may be of any suitable construction and material so long as the construction and material provide for one or more flexible lumen. In some embodiments, the tubing may be of any suitable construction and material so long as the construction and material provide for one or more lumen that are substantially, non-compressible. In some embodiments, polyurethane, polyether ether ketone, or nylon is used to construct the outer wall. In some embodiments, the outer wall may further comprise a mesh of stainless steel. In some embodiments, the catheter's outer diameter no more than 8 French. The outer diameter of the catheter's end section is also preferably no greater than about 8 French. In some embodiments, the catheter features an inner stiffening tube made of any suitable material. In one embodiment, the inner stiffening tube is constructed from polyimide. In a preferred embodiment, the catheter is compatible with a conventional guide sheath. In some embodiments, the catheter features a compression coil and puller wire assembly. In some embodiments, one or more coaxial electrodes are mounted directly to the distal end of the flexible tubing of the end section. In some embodiments, one or more ring electrode is mounted to the end section of the catheter and connected to lead wires.

In some embodiments, the catheter end section comprises a deflectable segment and an adjustable circle or loop housing an electrode array of variable number. In some embodiments, the catheter electrodes are irrigated electrodes and perfusion fluid channels are incorporated in the main body portion, and the control handle. In some embodiments, a compression coil surrounds the puller wire from the proximal end of the catheter tubing to the proximal end of the end section. In some embodiments, the compression coil is made of stainless steel.

The current invention, therefore, provides a catheter suitable for use for inducing cells in vivo to induced potency by application of an electrical field to a target cell or tissue; said catheter will also be suitable for injection of an agent, especially a marker, therapeutic, drug, transfectant, chemical, or compound into a target tissue, organ or cavity; for in vivo reversible electroporation; and for in vivo irreversible electroporation.

The ability to derive proliferating, self-renewing, multipotent, pluripotent, and totipotent cell population(s) from otherwise non-pluripotent, non-self renewing cells has significant positive implications for all fields utilizing cellular therapies, particularly in the absence of requisite reprogramming factors. These fields include bone marrow transplantation, transfusion medicine, and gene therapy and enable the production of patient-specific stem cells and other desired cell types. Likewise, the ability to initiate differentiation of cells other desirable cell populations has significant value to medicine and commercial processes involving animals.

The present invention provides methods for developmentally-activating, a cell to without the requisite use of nucleic acid or protein factors. Moreover, the methods of the present invention are cost effective, reliable, easily optimized to multiple purposes and easily scalable. The methods taught herein, likewise, avoid requisite use of oncogenes and undesirable genomic alteration.

A relatively new technology known as flow electroporation (flow EP) has emerged over the last decade and is capable of uniformly transfecting as many as 200 billion cells in 30 minutes. Moreover, flow EP shows broad compatibility with respect to cell type and has been optimized to accommodate a large number of cell morphologies. The use of Flow Electroporation to in the methods described herein is specifically covered by the present invention. The maturation of microfluidic technologies and nano-manufacturing methods has been critical to the successful development of flow EP by reducing the required fluid volume, shrinking the space between electrodes (gap width) from millimeters to microns (Wang and Lu, 2006, 2008; Wei et al., 2011; Geng and Lu, 2013; Zhao et al., 2016; Gencturk et al., 2017), and reducing the voltage requirement. As a consequence, flow EP exhibits transfection efficiency and cell viability characteristics useful for scaling developmental cell activation, consequently, the present invention simultaneously provides methods for inducing multipotency, pluripotency r totipotency in a cell not previously displaying it without use of nucleic acids. The present invention also provides methods do not require nucleic acids, protein factors or small molecules; nevertheless, nucleic acids, proteins and/or small molecules may be used as adjuncts in the methods taught herein to increase, for example efficiency and/or speed. Such adjunctive use is specifically covered by the present invention.

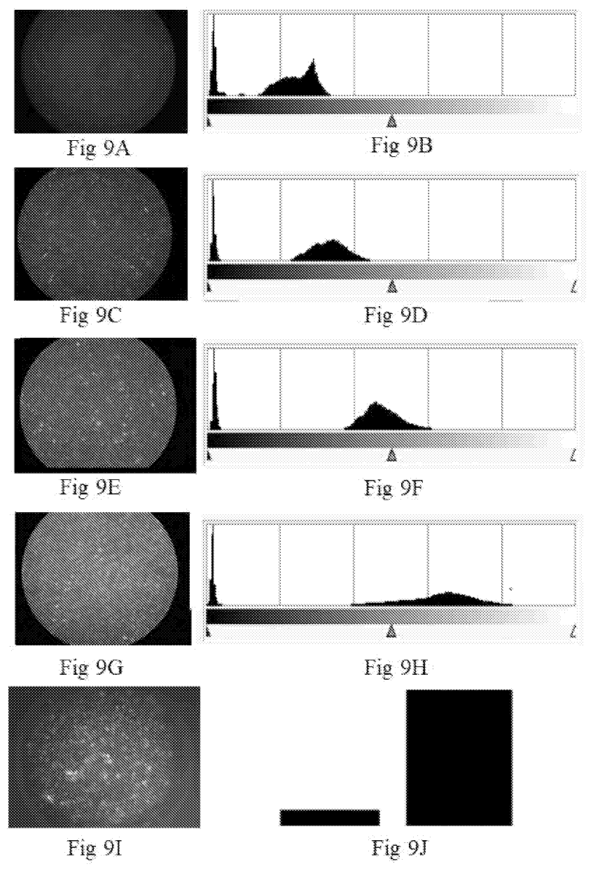

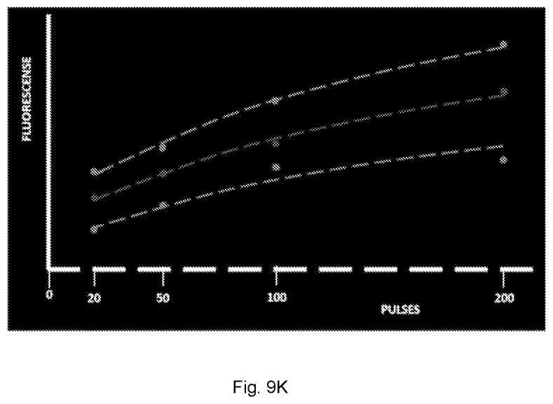

Both magnetoporation and electroporation have been used successfully in vivo and in vitro. The experiments depicted in FIG. 9 shows they achieve similar performance with similar pulse number. Accordingly, the two methods are proposed herein to be used, in various embodiments, interchangeably. Sonoporation and Optoporation are likewise practicable in the present invention. Some exemplary equipment, methods, protocols, and compositions relating to sonoporation, magnetoporation, optoporation and electroporation, as well as algorithms and strategies for adapting static electroporation protocols to flow electroporation, are useful in various embodiments of the invention and are described in the following articles, books, patents and patent applications and are incorporated in their entireties herein: Novikij et al., (2020); Li et al., (2008); Zhou et al., (2007); Pereyra et al., (2016); Almstaetter et al., (2015); Akiyama et al., (2010); Hashimoto & Hisano, (2011); Sapet et al., (2012); Soto-Sanchez et al., (2015); Mason, (988); Kim et al., (1996); Miller and Song, (2003); Mehier-Humbert and Guy, 2005); Lu et al., (2003); Koike et al., (2005); Miao et al., (2005); Delalande et al., (2013); Delalande et al., (2013); Electroporation Protocols, edited by Shulin Li; T. Geng et al., (2011); D. Zhao et al., (2016); Gaebler, et al., (2020); Y. Cao et al. (2019); Chicaybam et al., (2017); Sherba et al., (2020); Potter and Heller (2003); Li et al., (2002); Craiu et al., (2008); Parham et al., (1998); Wang et al., (2009); Wang et al., (2010); Li et al., (2013); Wei et al., (2011); Kamigaki et al., (2013); and Steger et al., (2015); Dijkink et al., (2008); Fan et al., (2013; 2014, 2015); U.S. Pat. No. 7,029,916B2; BTX Optimization Guide; US721124; U.S. Pat. Nos. 5,677,139; 6,432,711; 5,453,357; 5,593,875; 5,783,566; 5,928,944; 5,910,488; 5,824,547; 8,921,332; U.S. Pat. Application 20180028567; U.S. Pat. No. 5,925,565; U.S. Pat. No. 20180112235; U.S. patent Ser. No. 10/435,713; U.S. Pat. Nos. 8,110,360; 5,464,764; U.S. Pat. No. 20180053547; U.S. patent application Ser. No. 10/080,272; U.S. Pat. Nos. 6,485,961; 4,683,202; U.S. patent Ser. No. 13/902,444; U.S. Pat. No. 4,366,241; U.S. Pat. No. 20110236962; U.S. Pat. Nos. 5,627,059; 6,150,148; 6,074,605; 4,857,451; 6,204,061; U.S. Pat. No. 20160281047; U.S. Pat. No. 20040171156; U.S. Pat. No. 20120277120; U.S. Pat. No. 20180023045; U.S. Pat. Nos. 6,277,608; 6,773,669; 6,617,154; U.S. Pat. No. 20070231873; U.S. Pat. Nos. 7,422,889; 5,654,182; 8,569,041; 9,063,136; U.S. Pat. Application 20170258837; U.S. patent application Ser. No. 10/399,364; U.S. Pat. No. 20130196441; U.S. Pat. Nos. 6,074,605; 9,534,989; WO03/018751; U.S. Pat. Nos. 7,029,916; 3,996,345; 5,545,130; U.S. patent application Ser. No. 10/316,335; U.S. Pat. No. 20130253040; U.S. patent Ser. No. 12/421,352; U.S. Pat. Nos. 9,988,624; 4,661,913; U.S. Pat. Application 20120088842; U.S. Pat. No. 20110009807; U.S. Pat. Nos. 9,132,153; 5,134,070; 5,935,819; U.S. Pat. No. 20050118705; U.S. Pat. Nos. 7,141,425; 8,153,432; U.S. patent application Ser. No. 14/834,932; U.S. Pat. No. 3,850,752; U.S. patent application 20040214333; U.S. Pat. No. 20170316353; U.S. patent Ser. No. 10/017,760; U.S. Pat. No. 5,677,177; U.S. Pat. No. 20170218355; U.S. Pat. Nos. 6,090,617; 5,888,732; U.S. patent application Ser. No. 10/225,446; U.S. Pat. No. 20170307606; U.S. Pat. No. 20030104588; U.S. patent application 20040197883; U.S. Pat. Nos. 6,773,669; 8,667,840; 8,450,112; 9,546,350; U.S. patent application Ser. No. 10/781,440; U.S. Pat. No. 9,669,058; U.S. patent application Ser. No. 14/834,932; U.S. Pat. No. 8,677,839; U.S. Pat. No. 20110213288; U.S. Pat. Nos. 7,771,984; 7,991,559; U.S. Pat. No. 20060224192; U.S. Pat. Nos. 4,714,682; 8,332,160; 9,029,109; 7,771,984; U.S. Pat. No. 20140350456; U.S. Pat. No. 20110061474; U.S. Pat. Nos. 6,617,154; 4,833,080; 7,112,715; 3,817,837; 9,738,918; 6,746,441; 5,885,836; U.S. Pat. No. 20040115784; U.S. Pat. No. 20180179485; U.S. Pat. Nos. 9,593,359; 6,077,479; U.S. Pat. No. 20160018427; U.S. patent application Ser. No. 10/225,446; U.S. Pat. Nos. 5,676,646; 4,220,916; U.S. patent application Ser. No. 10/080,272; U.S. Pat. No. 5,098,843; U.S. Pat. No. 20170283761; U.S. Pat. Nos. 5,612,207; 6,509,156; U.S. Pat. No. 20110082093; U.S. Pat. No. 4,275,149; U.S. patent application Ser. No. 10/675,592; U.S. Pat. Nos. 4,277,437; 8,450,112; U.S. Pat. No. 20160298074; U.S. Pat. No. 20120156786; U.S. Pat. No. 5,487,992; U.S. patent application Ser. No. 12/421,352; U.S. patent Ser. No. 10/443,074; U.S. patent application Ser. No. 10/675,592; U.S. Pat. No. 20030073238; U.S. Pat. No. 20150297887; U.S. patent application Ser. No. 11/127,557; U.S. Pat. Nos. 8,932,850; 8,697,359; 6,773,669; U.S. Pat. Application No. 20030059945; U.S. Pat. No. 20110065171; U.S. Pat. No. 7,186,559; U.S. patent application Ser. No. 13/902,444; U.S. Pat. Nos. 5,478,722; 7,186,559; 8,726,744; U.S. patent application Ser. No. 10/781,440; U.S. Pat. No. 9,361,427; U.S. Pat. No. 20070105206; U.S. Pat. No. 6,774,279; U.S. Pat. No. 20180169148; U.S. Pat. No. 7,141,425; U.S. Pat. No. 20160333302; U.S. Pat. No. 20170029805; U.S. Pat. No. 20080138877; U.S. Pat. Nos. 5,720,921; 4,498,766; 8,758,623; 6,143,527; 5,631,153; U.S. Pat. No. 20050064584; U.S. Pat. Nos. 4,989,977; 6,090,617; 5,160,974; 4,767,206; 6,090,617; 8,677,840; 8,584,535; U.S. Pat. Application 20200237825; U.S. Pat. Nos. 4,284,412; 9,132,153; 5,928,906; 9,790,490; 8,110,122; PCT/US2014/028561; U.S. Pat. application 20180112235; U.S. Pat. Nos. 3,826,364; 6,916,632; 4,774,189; U.S. Pat. No. 20180028567; U.S. Pat. No. 20070249036; U.S. Pat. Nos. 7,029,916; 8,584,536; 9,982,279; U.S. Pat. No. 20160272961; U.S. Pat. Nos. 8,450,112; 6,956,146; WO 2004/031353; U.S. Pat. Nos. 6,482,619; 6,689,610; 3,939,350; 9,896,696; 4,946,793; U.S. Pat. No. D731634; U.S. Pat. No. 20170067007; U.S. Pat. Nos. 5,137,817; 6,654,636; U.S. Pat. No. 20140121728; and U.S. Pat. No. 4,959,317. To the extent that they provide exemplary procedural or other details supplementary to those set forth herein, these patent, patent applications, books and articles are specifically incorporated herein. For example, T. Geng et al., (2011) describes means by which static electroporation protocols may be translated to flow electroporation protocols effective in the present invention,
“instead of using a pulse generator, the applicants use a common power supply that applies a constant voltage across a fluidic channel . . . . The local field intensity and the cross-sectional area of a particular section are inversely proportional . . . the channel is typically made of uniform depth and composed of wide and narrow sections in order to create variation in the cross-sectional area. Appropriate combination of the voltage and the channel design yields high field intensity in the narrow sections and low intensity in the wide sections.”

“The field intensity in the narrow sections is beyond the electroporation threshold, whereas the intensity in the wide sections does not affect membrane integrity. Cells are exposed to the Electrical field while flowing through the narrow section(s) and the duration of electroporation is determined by the residence time(s) in the narrow section(s).”

The applicant demonstrates that application of energy in the form of an electrical field (electroporation, 300V, 30-70 pulses, 5 ms pulse length, 100 ms pulse interval) to fibroblast or buccal cheek cells under in the absence of nucleic acid or protein factors, provided for rapid induction of pluripotency (within 24-72 hours) which was durable and maintained effectively in standard cell culture media containing DMEM and supplemented with bFGF; nevertheless, the applicants observed that the efficiency (induced cells/target cells) was increased when an Electric field was applied in the presence of Oct4, Sox2 and Nanog proteins (50 ug per protein). Similar poration has been achieved in the presence of EtBR using magnetoporation with a generator capable of producing field pulses up to 5 T with rise time in microsecond range and operating up to 40 Hz (FIGS. 9I and J).

In some embodiments, the Electrical field is applied (e.g. by electroporation or magnetoporation) to cells at 4C, or on ice.

In nature, pluripotency and multipotency tend to be transient states. However, exogenous nucleic acids and proteins previously taught by others and by the applicant tend to act over time, thereby prolonging multipotency or pluripotency in reprogrammed cells. Likewise, small molecules and cell culture agents may act to prolong pluripotency or differentiated cell states, in vitro. Accordingly, the use of these adjuncts and others is specifically covered in alternate embodiments by the present invention. For example, in some embodiments, a cell induced to pluripotency by application of energy in the form of an electrical field (e.g. magnetoporation or electroporation) in the absence of exogenous nucleic acids or proteins factors, may be further induced to a desirable differentiating or differentiated cell type by application of Small RNA and/or proteins corresponding to transcription factors and/or other cell fate determinants normally expressed in the desired cells. Said proteins transcription factors and/or other cell fate determinants may likewise be introduced into the cell of induced potency utilizing electroporation.

It is a proposition of the present invention that the efficient application of Energy to nucleated cells in the form of an electrical field or charge (z) promotes developmental activation observed through changes in global gene expression characterized by one or more of a. rapid induction to pluripotency (within 24-72 hours); b. induced expression of a pluripotency or totipotency reporter gene; c. enrichment of pluripotency or totipotency associated gene expression; d. adoption of cell morphology consistent with pluripotency or totipotency; e. embryoid formation or embryo formation consistent with pluripotency or totipotency; f. hierarchical clustering with or among pluripotent or totipotent cells relative to somatic cells in Heatmaps; and g. clustering with or among pluripotent or totipotent cells relative to somatic cells in Principal Component Analysis plots.

Energy may be applied to cells using various modalities, e.g. magnetoporation, sonoporation, and optoporation, according to methods well known in the art—each having distinct advantages, for the purpose of developmental activation and such alternative means of application is covered by present invention.

The ability to reliably induce developmental activation, e.g. phenotypic conversion, allows the production of stem-like cells, replacement cells, tissues, and organs that match individual patients or subjects. In conjunction with gene therapy techniques, protein therapy techniques, cellular cargo loading, and cell culture techniques, cell type interconversion, also provides for the production of disease-resistant and genetically-repaired cells that are suitable for transplantation and organ production. The current invention teaches that it is the particular complement of transcription factors within an individual cell that determines which cellular programs are active and which are turned off. In this capacity transcription factors play a decisive role in determining and maintaining cellular identity, as well as determining cellular vulnerability. The applicants teach herein that the application of Energy in the form of an electrical field or charge (z), has the ability to alter the expression of said transcription factors and cell fate determinants. To the extent the application of electrical charge induces cells to pluripotency, said induced pluripotent cells can be termed charge (z)-activated, pluripotent cells.

It is a further teaching and proposition of the present invention that cells may be activated to self-renewal, multipotency, pluripotency, and/or totipotency in the absence or presence of Small RNAs, especially Small RNAs the applicants identified by global gene expression analyses as being enriched in charge activated, pluripotent cells. Using transcriptome analysis, the applicants identified numerous transcription factors, kinases, small RNAs and other cell fate determinants enriched in human and/or murine developmentally-activated cells. (See Example 45 and Tables 1-3).

Likewise, the applicants have identified certain small molecules as having the ability to mimic the characteristic patterns of gene expression demonstrated in Applicants' developmentally-activated cells; such small molecules are also taught herein for producing or enhancing developmental activation, alone, in combination with one another, and/or in conjunction with other effectors of developmental activation described herein.

In some embodiments, the small molecules so identified, and/or other small molecules known to those skilled in the art may also be utilized as adjuncts in “factor free reprogramming” and are specifically covered in alternate embodiments by the present inventions. It is an object of this invention to provide various manners of generating developmentally-activated cells (DAdC) that may be useful in the role of proliferating, self-renewing, multipotent, pluripotent, totipotent and/or differentiating cell population(s). Differentiating cell populations (aka differentiating somatic cell populations) include cells expressing some, but not all markers associated with specific cell type categorization. In some preferred embodiments, the totipotent cells of the present invention are non-human totipotent cells. It is disclosed herein that the application of Energy in the form of an electrical field or charge enables the production of cells with characteristics normally associated with totipotent, pluripotent, or differentiating cell populations. Similarly, it is taught herein that the application of Energy in the form of an electrical field or charge enables induction of proliferation, self-renewal, or stem/progenitor cell behavior in endogenous cells in vivo.

Likewise, the equipment, methods, and compositions of the present invention may be used block proliferation, self-renewal, or stem/progenitor cell behavior in cells aberrantly displaying such behavior (e.g. cancer cells). It is also an object of the present invention to provide therapeutic vectors for use in said Energy or charge activated cells which are capable of expressing beneficial sequences (such as small RNAs or synthetic oligonucleotide sequences, or sequences coding for proteins) predicted to attenuate disease processes.

The current invention specifically claims the combination of said therapeutic vectors and/or beneficial sequences in conjunction with the DadC (e.g. reprogrammed) cells of the present invention, including the charge activated or factor free induced pluripotent cells.

For example, the current invention discloses the use of synthetic oligonucleotides to reduce gene expression critical HIV and other immunodeficiency virus infection, propagation and spread. The present invention specifically covers anti-HIV synthetic oligonucleotides in conjunction with the DadC of the present invention, including the charge activated or factor free induced pluripotent cells.

The current invention likewise discloses the CRISPR/Cas9 related sequences or proteins in combination or conjunction with the DadC cells of the present invention, including the charge activated or factor free induced pluripotent cells.

### Equipment and Apparatuses Suitable for Use in Electroporation

The present invention relates in part to equipment and apparatuses suitable for use in electroporation (Shigekawa and Dower, 1988). Factors that can be varied to optimize electroporation effectiveness are discussed in introduction to Section I, and protein expression strategies are discussed in Chapter 16 of Curr Protoc Mol Biol. 2003 May; doi:10.1002/0471142727.mb0903s62.

A transfection high-voltage controller is taught by U.S. Pat. No. 4,750,100. The commonly employed practice of transferring cells from a first cell culture apparatus to a cuvette and then to another cell culture apparatus is attendant with contamination and infectious risks. The Neon® Transfection System is a second-generation transfection system that uses an electronic pipette as an electroporation chamber, but Neon still requires that the electroporation procedure be carried out within a sterile environment, e.g. a restrictive, cell culture hood, and does not completely eliminate the transfer related contamination and infectious risks.

In contrast, the present invention provides a combined electroporation chamber/cell culture apparatus allowing application of the Electrical Field and cell culture to be accomplished in the same cell culture apparatus-obviating the need for transfer from an electroporation chamber to a separate cell culture apparatus (e.g. via magnetoporation or electroporation).

The novel culturing apparatus (assembly) for developmentally-activating a cell of the present invention may be termed a cell culture dish. More particularly, the present invention is a “combined cell culture dish” or “dish-in-dish” apparatus for developmental activation, e.g. by magnetoporation or electroporation, comprising at least one smaller cell culture dish fixedly positioned within a larger cell culture dish, and the number of such fixated cell culture dishes can include a multiple number of fixated cell culture compartments within one another, either concentric or eccentric, in any number of geometric shapes, and without limitation to the number of compartments included. An alternate embodiment of this invention can include a plurality of cell culture dishes juxtaposed side-by-side having common interior well walls, and the well walls may or may not be different in height depending on the application. The combined cell culture dish differs from the prior art, in part, because the walls of said combined compartments may be of different heights and made from any combination of transparent and non-transparent materials that will allow juxtaposing cultures to grow simultaneously.

The combined cell culture/developmental activation apparatus of the present invention may or may not be fitted with single or multiple covers and may or may not be stacked. A particular embodiment of the arrangement described herein comprises one or more smaller dish or compartment (aka reservoir or reservoirs) located inferiorly, within, or adjacent to a larger cell culture dish with which it can communicate, and wherein said smaller dish (which may have dimensions akin to those of standard electroporation cuvettes) comprises electrodes or electrode plates that enable electroporation. In some embodiments, a wall of low height (aka a lip) will separate the smaller dish/compartment (e.g. the cuvette-like reservoir) from the larger dish/compartment. The low wall or lip and/or funnel demarcates and surrounds the reservoir while also demarcating the space by which these two cell cultures may communicate (e.g. if a sufficient volume of medium is added and the cuvette-like reservoir overflows). Such an arrangement of compartments allows cells to undergo developmental activation by electroporation and incubate in a single cell culture apparatus-obviating the need to transfer cells from a first apparatus (e.g., a first cell culture dish) to a second apparatus (e.g., an electroporation cuvette), as well as obviating the need to transfer the cells from the second electroporation chamber apparatus to a third apparatus (e.g., a second cell culture dish) for further incubation; accordingly, this particular arrangement provides a “closed system” that reduces labor, costs of materials, and infectious/contamination risks.

The main dish, plate, flask, or compartment and the reservoir may be of various sizes and dimensions. In some embodiments, when the design features a flask-like compartment, the flask like compartment will preferably approximate standard flask dimensions, while the reservoir may approximate standard electroporation cuvette sizes. In one embodiment, the flask, plate, dish, bag, etc. cell compartment takes the width of an embedded cuvette-sized reservoir.

In one embodiment, the reservoirs are detachable and snap onto or slide into the larger main dish, plate or flask. In some embodiments, the cell culture compartment communicating with a reservoir suitable for electroporation is a cell culture bag. In some embodiments, the cell culture compartment communicating with a reservoir suitable for electroporation is a bioreactor of variable dimensions and shapes. Accordingly, one skilled in the art will recognize that the cell culture compartment may be of any construction, shape or size, so long as it may be made to communicate with a second compartment of variable size and construction suitable for application of an Electrical field. By the same token, the second compartment may be of any construction, shape or size, so long as it is suitable for electroporation. By the same token, any Flow Electroporation system communicating with a cell culture compartment (e.g. cell culture bag) is hereby covered by some embodiments of the present invention. Likewise, the spatial relationship between the multiple compartments taught herein allows the application of an Electrical field to be performed in a non-sterile environment, e.g. outside of the tissue culture hood, at the bench.

Currently, the size of a tissue culture hood limits the size of the such apparatuses. When performed outside of the tissue culture hood using the cell culture dishes, plates and flasks equipped with one or more reservoirs, as taught herein, the magnetoporation/electroporation apparatus may be of unlimited size and can be used to perform electroporation of multiple (up to hundreds or thousands of) cell cultures simultaneously, thereby enabling higher throughput. Accordingly, a parallel array of electrodes suitable for high throughput, parallel electroporation is also described herein. Such an array may take the form of a slot or slots containing multiple coils for magnetoporation or electrode pairs for electroporation spaced at distances accommodating the dimensions and spacings of the reservoirs, or a block with multiple wells, each well containing one or more coils or electrode pairs and having dimensions accommodating the one or more reservoir portions of the “reservoir-in-dish” or “reservoir in flask”, etc., cell culture dishes, plates, flasks, bags, bioreactors, etc.

In general, the cell culture magnetoporation/electroporation apparatus of the present invention comprises two or more compartments which create a central compartment and one or more peripheral compartments which surround the central compartment. Said central and peripheral compartments may take the form of any shape, or any geometrical relationship including, but not limited to cylindrical, square, pentagonal, or hexagonal. The material used to construct said petri dish may include, but may not be limited to any non media-permeable form of glass, plastic or metal or combination thereof, which will sustain culture growth and permit observation and recording of said culture growth, differentiation and/or signal transduction. Separated areas created by utilizing the central compartment and one or more peripheral compartments may be geometrically concentric or eccentric.

The cell magnetoporation/electroporation apparatus (with dimensions suitable for either electroporation or magnetoporation) depending on the embodiment, (Novickij et al., 2013) may comprise one or more compartments within a compartment or may be constructed of a single compartment with a flat well bottom having one or more sets of walls that extend from said well bottom forming one or more separate enclosures having the same geometric shape or a variety of geometric shapes.

In some embodiments, the walls of the combined cell culture/electroporation apparatus are arranged in a manner that allows communication of cells and/or media between and amongst the separate compartments when a sufficient volume of medium is present. For example, one compartment may be filled with cells and/or medium to a certain height, wherein the medium and/or cells remain restricted, confined or localized to a first compartment, and wherein further addition of cells and/or medium allows the contents of the first compartment to ascend above or spill over walls or lips demarcating one compartment from a second compartment, or to spread into a second compartment communicating with the first.

It is taught further herein that the application of an Electrical field (e.g. via electroporation or magnetoporation) is capable of successfully activating a cell, either directly or indirectly to a desired cell type with greater speed, greater efficiency and greater safety than with previously taught methods.

It is further taught herein that adjunct use of a protein, nucleic acid, or other factor known to those skilled in the art may likewise serve to developmentally-activate a cell, either directly or indirectly (Srivastava and DeWitt, 2016; Seo et al., 2017; Fan et al., 2018; Kogut et al., 2018; McGrath et al., 2018; and Aydin and Mazzoni; 2019) and may be applied (in vivo or in vitro) to achieve activation with greater speed, greater efficiency and greater safety than with known methods.

It should be understood that “transitory” type DAdC can also serve as selected cells and be converted to “fixed” DAdC according to the methods described herein, as well as according to methods published elsewhere and known to the art for converting pluripotent, multipotent, or pluripotent-like cells to various differentiated cell types.

While the text herein refer either only to “introduction” or only to “overexpression”, it is to be understood that causing a cell to overexpress a gene has, in the context of the present invention, the same effect as introducing said gene or corresponding RNA or corresponding protein into said cell; and accordingly, the associated methods for introducing or overexpressing can be used interchangeably in practicing the invention.

Some small RNAs are suitable and compatible with use in the invention and include small RNAs useful for achieving proliferating, self renewing, pluripotent, and/or pluripotent-like cells; these small RNAs include one or more selected from the miR-302/367 cluster small RNAs (miR-302a, miR-302b, miR-302c, miR-302d, miR-367), human miR-371-373 cluster small RNAs (miR-371, miR-372, miR-373), miR-17-92, C19MC cluster members, miR-133b, miR 200a, miR 23a, and miR 743b-5p, miR-187, 299-3p, 499-5p, 628-5p, miR-888, let-7 (let-7-b,e,f,g), miR-30 (miR-30-a-e), the mouse miR-290-295 cluster small RNAs (miR-290, miR-291a-3p, miR-291b, miR-292, miR-294, miR-295, miR-29, miR-296 and other pluripotency associated small RNAs known to the art, as such small RNAs can be used in conjunction with other cell fate determinants taught herein or alone. Use of RNA and proteins, which do not integrate into the host's genome, may be considered as safer approach to developmental activation as compared to other methods that pose the risk of genomic integration. Such vectors are considered to be non-integrating and/or episomal vectors.

Accordingly, use of chemicals, compounds, extracts and drugs that induce expression of said small RNAs and other cell fate determinants is likewise suitable and compatible with the present invention-especially those chemicals, compounds, extracts and drugs taught in the priority documents associated with the present invention.

Studies Relevant to Protein Transfectants and Distinguishing the Electroporation Method

Kim et al. 2009 and Zhou et al. 2009 reported that pluripotent cells could not be produced with high efficiency or using a single application of cell penetrating proteins corresponding to pluripotency inducing factors. In contrast the current invention, teaches, in part, adjunctive use of native or recombinant protein transcription factors and/or protein cell fate determinants for the production of developmentally activated cells, including totipotent and pluripotent-like cells, pluripotent cells, and/or self-renewing cells, as well as differentiating cells that express one or more cell type specific markers consistent with a desired cell type.

### Teaching Away

Based on the studies of Kim et al. 2009 and Zhou et al. 2009, those skilled in the at concluded that cell reprogramming, direct reprogramming, and pluripotency induction cannot be achieved using a single application proteins. The consensus view was that proteins introduced to cells for that purpose are too quickly degraded within the cells, consistent with their short half-lives under normal conditions.

For example, Seo et al. (2017) wrote, “Cell-penetrating peptide-based reprogramming might be a safe way to induce reprogramming; however, its low efficiency compared with other methods is a significant concern. The main problem is the poor stability of the recombinant proteins and following endocytic uptake”.

Dey et al., (2017) make similar observations to Seo with regard to the deficiencies associated with CPP-mediated protein delivery, stating,
“Presence of CPPs in reprogramming proteins is known to interfere with proper folding inside the cells and thereby decreasing the biological activity . . . endosomal entrapment is also a common barrier and is a major challenge in efficient delivery of CPP linked molecular cargo . . . . In a cell reprogramming paradigm to generate iPS cells via CPP-mediated recombinant protein transduction, reports also show that misfolded CPP-fused recombinant reprogramming proteins after endosomal release gets localized to cytoplasm and/or have a peri-nuclear region as observed in immunostained images [11,35,44,45,47-52]. Due to this, they are unable to enter the nucleus to activate downstream target genes.”

Ryu et al., (2020) likewise highlight the persistent long held need for cell activation, e.g. reprogramming methods that are not subject to the risks limiting other methods, writing,
“Recent studies have shown that fibroblasts can be directly reprogrammed into iNCs by forced expression of transcription factors under co-culture condition, especially by Ascl1, the key driver of iNC reprogramming (Vierbuchen et al., 2010; Pang et al., 2011; Chanda et al., 2014) . . . . However, the methods utilized genetic materials and/or potentially mutagenic molecules to generate iNCs. They could potentially be tumorigenic via integration of genetic material into the genome of host cell (Maherali and Hochedlinger, 2008). Protein-based approaches could solve those safety issues.

The methods herein teach away from Zhou et al., (2009); Kim et al., (2009), Dey et al (2017), and Seo et al., (2017), and address the long held need identified by Zhou, Kim, Seo and Dey, as well Ryu, et al (2020), The current invention teaches that a single application of protein electroporation to somatic cells at various voltages and pulse numbers rapidly produces developmental activation to a desired phenotype. Likewise, other transfectants, whether DNA, RNA chemical, small molecule, or proteins, can, according to the present invention, be applied either all at once in one round of electroporation, or via multiple rounds of electroporation. Moreover, the application of an Electric field by electroporation, magnetoporation, sonoporation, optoporation, etc., as taught herein, can be combined with a second transfection method in one or more separate rounds of transfection

Accordingly, the applicants teach herein methods involving adjunctive use of nucleic acids (less preferred) and proteins or peptides (more preferred) to increase the efficiency of developmental activation produced by electroporation alone; said teachings are contrary to the teachings of Zhou et al., (2009) and Kim et al., (2009). Applicants' teachings include various efficient protein-based methods for developmental activation that do NOT require multiple applications of the protein; these methods are likewise applicable to Small RNA named herein, chemicals and small molecules named herein, as well as DNA and RNA corresponding to genes named herein. In so doing, the applicants also address long held needs for safe and efficient methods of activation.

Moreover, applicants' adjunctive protein-based methods and small RNA based methods of cell activation address a long-held need for more efficient and safe methods for developmental activation; applicants' methods are safer in that they do not involve requisite use of nucleic acids. oncogenes or HIV based lentiviruses, or risk genomic alteration due to integration of viruses (Takahshi et al., 2007); applicants' methods are also more efficient than prior methods of developmental activation. Consistent with this teaching, applicants have observed up to 100% efficiency in their experiments, approximately 15,000× higher than the efficiencies achieved by Kim et al., (2009) and Zhou et al (2009).

In contrast to the experience of Kim et al., (2009) and Zhou et al., (2009), the current invention teaches that specific proteins may be delivered to the interior of cells in amounts such that the protein(s) persist in the cell long enough to cause the cells to become developmentally activated to pluripotency.

Int appears we achieved protein based activation, in part, by saturating the cell's intrinsic, protein-degradative pathways through application of Energy in the form of an Electrical Field (e.g. magnetoporation or electroporation) to cells, as well as with adjunctive introduction of excess protein reprogramming factors along with electroporation, thereby artificially extending protein half-lives in the cells. Proteins persisting longer in the cell are able to access their binding sites and interaction partners in the treated cells for longer periods of time.

The present invention teaches that half-life of transfected RNA may likewise be extended by means of electroporation, wherein large amounts of RNA are introduced into a cell saturating cellular RNA degradation pathways, similarly allowing the RNA species to persist longer within the electroporated cell.

The present invention further teaches that transfectant half lives (whether protein or nucleic acid, small molecule, or chemical) may be extended by electroporation, as well as by other transfectant delivery methods taught herein.

The current invention covers electroporation, and all other methods known to the art, capable of delivering the desired transfectants to the interiors of cells in amounts that are 1. sufficient to saturate degradative pathways to promote persistence of the proteins in the cells, thereby producing the desired effect, but simultaneously 2. insufficient in amount to kill the cells. As taught herein, there need be no damage to the cells or their membranes.

Numerous such methods are known to the art and are easily adapted by (e.g. by increasing protein or nucleic acid transfectant concentration); many such methods are taught herein; they include, for example, liposomal transfection methods, fusogenic or non-fusogenic liposomes, lipofectamine, cationic lipids (e.g. Thermo Scientific Pierce Protein Transfection Reagent (formerly Pro-Ject), and use of nanocapsules or nanovaults,

Accordingly, in some embodiments, the proteins adjunctively introduced or overexpressed in selected cells consist of recombinant proteins or nucleic acids, rather than natural protein extracts.

Likewise, in some embodiments wherein a permeabilizer akin to SLO is used to permeabilize cells for introduction of protein, the proteins introduced following permeabilization may consist of recombinant proteins. In some embodiments wherein a permeabilizer akin to SLO is used to permeabilize cells for introduction of protein, the proteins are not derived from cell extracts.

In some embodiments wherein proteins are introduced into cells to produce developmental activation, e.g. to totipotency, multipotency, pluripotency, and/or self renewal, the proteins introduced will comprise or consist of one or more of ZCAN4, PPP1R14A, DNMT3B, ZFP42, EPCAM, OCT4, CXCR4, SOX2, PRR+ NUMB, NOTCH, NANOG, HOXB4, and/or a gene with LIF activity. In some embodiments of the present invention, when proteins are introduced into cells, the proteins do not comprise a complete cellular protein extract.

In some embodiments, when proteins are introduced into cells, the proteins do not comprise a complete cellular extract derived from a cancer cell or embryonic stem cell.

In some preferred embodiments, when large throughput is desired, the method of electroporation is flow electroporation (Li et al., 2002; Craiu et al., 2008; Parham et al., 1998; Wang et al., 2009; Wang et al., 2010; Li et al., 2013; Wei et al., 2011); Kamigaki et al., 2013; and Steger et al., 2015; U.S. Pat. No. 7,029,916B2).

The invention further covers the use of cell penetrating peptides in conjunction with electroporation or another delivery method that increases the amounts and efficiency with which the peptides enter the cell, as described above.

The present invention covers the combination of various delivery methods, such as electroporation in conjunction with liposomal protein, nucleic acid or other molecule delivery; electroporation in combination with cell penetrating peptides or other recombinant proteins; electroporation in combination with viral transduction; electroporation in combination with nanoparticle, nanotube, nanocapsule or nanovault delivery; electroporation in combination with cationic lipids; electroporation in combination with non-integrating viral vectors (e.g. integrase deficient, episomal, lentiviral vectors); cationic lipids in combination with nanoparticle, nanotube, nanocapsule or nanovault delivery; cationic lipids in combination with cell penetrating peptides, etc. The invention further covers the use of other methods and reagents such as those described in U.S. Pat. No. 6,841,535 for the delivery of the protein(s) and other molecules taught herein. We have successfully induced millions of cells, at high efficiencies, to change morphology, form colonies, form embryoid, express markers of pluripotency, and display reactomes consistent with developmental activation using a single application of electroporation (see Koken et al., 1994) and other methods described herein.

However, the invention in no ways precludes repeated application of proteins, nucleic acids, small RNAs or other cell fate determinants taught herein.

In some preferred embodiments, in order to produce developmentally-activated cells (DAdC), selected cells are transferred to a 4 mm gap cuvette and are electroporated using voltages ranging from ˜100V to 1300V (preferentially ˜300V) and pulses ranging from ˜10 to 700 pulses (preferentially 50-200 pulses), and preferably a pulse length of ˜2-20 ms with pulse intervals of ˜20 to 1 s, and are then cultured under standard cell culture conditions or pluripotent promoting cell culture conditions.

In one preferred embodiment, in order to produce developmentally-activated cells (DAdC), protein transcription factors and small RNAs and/or other cell fate determinants are electroporated into selected cells using pulse length of ˜2-20 ms and pulse intervals of ˜100 ms.

However, any electroporation protocol known to the art and suitable for efficiently introducing into selected cells, nucleic acids or proteins corresponding to transcription factors, small RNAs and/or other cell fate determinants, is practicable in the invention.

In one embodiment, developmentally activated cells are produced by electroporation with one or more transfectant selected from DNA, RNA, protein, small molecule, chemical, compound, extract, and/or oil. In one embodiment, developmentally activated cells are produced by electroporation of one or more transfectant selected from DNA, RNA and/or protein corresponding to one or more transcriptions factors and/or cell fate determinants.

In one embodiment, developmentally activated cells are produced by electroporation of one or more adjunct DNA transfectant, whether plasmid DNA, vector DNA, an aptamer, a synthetic oligonucleotide, or other source of DNA encoding or inducing or promoting expression of a transcription factor and/or other cell fate determinant. In one embodiment, developmentally activated cells are produced by electroporation of one or more adjunct RNA transfectant, whether naked RNA, an RNA virus, small RNA, miRNA, a synthetic oligonucleotide, an aptamer or other source of RNA translatable to or inducing or allowing expression of a transcription factor and/or other cell fate determinant.

In one embodiment, developmentally activated cells are produced by electroporation with one or more adjunct protein transfectant, whether a peptide, full length protein, partial protein, natural protein, native protein, synthetic protein, recombinant protein, or other source of protein acting as a transcription factor or other cell fate determinant; or inducing or allowing the expression of a transcription factor and/or other cell fate determinant.

In one preferred embodiment, developmentally activated cells are produced by electroporation alone, albeit at lower efficiencies, in the absence of an adjunct DNA, RNA or protein transfectant. In one embodiment, the one or more transfectants is derived from a subject's or a patient's own cells, tissues, fluids or body. In one embodiment, developmentally activated cells (DAdC) are produced using sonoporation (see Delalande et al. 2015; Wang et al., 2018), gene gun (see Sanford, 1993; O'Brie, 2001; O'Brien and Lummis, 2007), or laser based transfection (see Yao et al., 2008; Kim and Eberwine, 2010; Pylaev et al., 2018), or by these and other transfection methods (see Kim and Eberwine, 2010; Parent 20192019) or their combination via application of Energy to the selected cells in the absence of adjunctive nucleic acid or protein factors, and without damage to the cells or their cell membranes. In one embodiment, developmentally activated cells are produced by electroporation, albeit at lower efficiencies, in the absence of any transfectant other than the salts and other components of the buffer (e.g. of phosphate buffered saline).

It is the proposition of this invention that application of an Electric field allows safer and/or faster production of developmentally activated cells, is compatible with adjunctive DNA, RNA and Protein transfectants, and allows the avoidance, when desired, of integrating viruses, and/or reliance on oncogenes. It is the proposition of this invention that Application of an Electric field, e.g. by magnetoporation or electroporation, allows safer and/or faster production of developmentally activated cells, is compatible with adjunctive DNA, RNA and protein transfect ants, and that multiple rounds of electroporation are not required to achieve the desired effect; although the invention anticipates, contemplates and covers the possibility that some practitioners of the invention may, for example, decide to apply multiple rounds of electroporation.

It is the proposition of this invention that application of an Electric field allows safer, more efficient and/or rapid production of developmentally-activated cells without a requirement for special cell culture conditions; however the invention anticipates, contemplates and covers the use of special cell culture conditions known to skilled persons in the art that may further facilitate or enable the developmentally activated cells to acquire the desired cell phenotypes.

As taught herein, reprogrammed cells represent an example of developmentally-activated cells. Likewise, induced pluripotent, induced multipotent, induced self-renewing and/or induced somatic cell types (aka differentiating cells), as described herein, further represent examples of developmentally-activated cells.

A large number of protocols useful for electroporation of various transfectants into various cell types are known to those skilled in the art, for example many are archived at www.btxonline.com (https://www.btxonline.com/technical-resources/protocol-database.html; https://www.btxonline.com/media/wysiwyg/protocol_db/Electroporation_Optimization_Guide.p df; and https://www.btxonline.com/media/wysiwyg/protocol_db/General_Protocol.pdf). Likewise, these protocols are easily adaptable by those skilled in the art to flow electroporation.

We found that Delivery of the transfectant increases predictably with pulse number.

All settings that the applicants tried were successful in delivering protein to the interior of the cells selected. FITC-conjugated albumin served as a test transfectant and offered an excellent means of immediately visualizing the extent of protein delivery to the interior of the cells in conjunction with various electroporation parameters. The invention may be practiced in vivo using a variety of existing methods and equipment known to the art, However, the present invention also teaches a novel device for in vivo electroporation comprising a catheter and electrode(s).

In one embodiment, the catheter and electrode(s) are combined, with or without a camera and/or light a light source, as an assembly that can be optionally mounted on a wire or flexible tube such as are used for cardiac catheterization and for endoscopy. In some embodiments, the electrodes are sharp and capable of piercing tissue. In some embodiments, the electrodes are dull. In some embodiments, said assembly may also comprise a needle enabling injection of a transfectant into a tissue and a reservoir (e.g. syringe) where the transfectant is stored immediately prior to injection. In some embodiments, said assembly may comprise electrodes taking a form akin to “tweezertrodes”.

### Sources of Cells Selected for Use in the Invention

In a preferred embodiment, cells are “selected” from accessible, dividing or non-dividing cell populations for the purpose of generating or producing desired, developmentally-activated or cells. The desired activated cell may, for instance, be a) proliferating, multipotent, pluripotent or totipotent cells, or b) differentiating somatic cells; moreover the desired differentiating cell population may be capable of further differentiation in vitro, further differentiation in vivo, and/or tissue-appropriate and regionally-appropriate differentiation in vivo.

Selected cells may include any cell practicable in the present invention. Cells selected for use in the present invention (herein termed “selected cells”) may originate as endogenous cells of a subject or of a patient—including cells derived from other organ systems; or from exogenous sources (including those derived from cell lines, cryopreserved sources, banked sources, and donors). Cells may also be selected from cells genetically-modified with synthetic or natural nucleic acid sequences (or their corresponding proteins). The term “selected cells”, as used herein, does not include human embryonic stem cells.

In a preferred embodiment, the selected cells are somatic cells that do not display pluripotency or totipotency prior to treatment by the methods described herein. In a preferred embodiment, the selected cells are cells that are not immortalized. In a preferred embodiment, the selected cells are euploid In another preferred embodiment, the selected cells are genetically-normal. In a preferred embodiment, the selected cells are genetically-compromised. In a preferred embodiment, the selected cells are genetically-compromised, but subject to genetic correction according to methods taught herein or by other methods known to persons skilled in the art. In embodiments of the present invention, in order that they may be isolated without the involvement of invasive procedures, selected cells will preferably be easily accessible cells (e.g. peripheral blood leukocytes, circulating hematopoietic stem cells, epithelial cells (e.g. buccal cheek cells (e.g. Michalczyk et al., 2004), excreted cells, adipose tissue cells (e.g. Gimble et al., 2007; Ma et al., 2007), umbilical cord blood cells (e.g. Zhao, et al., 2006; Tian et al., 2007), etc.). However, bone marrow derived cells, stem cells isolated from amniotic membranes (e.g. Ilancheran et al., 2007), or amniotic fluid (e.g. De Coppi et al., 2007), as well as cells isolated from the skin (e.g. Tumbar, 2006; Dunnwald et al., 2001; Szudal'tseva et al., 2007), etc., are also covered by the present invention. Such cells can be isolated from the tissues in which they reside by any means known to the art.

The selected cells may be genetically-modified cells, especially cells that have been genetically modified by any means known to the art, to encode therapeutic or commercially useful deoxyribonucleic acid (DNA) or ribonucleic acid (RNA) sequences. The selected cells may be genetically-modified cells, especially cells that have been genetically modified by any means known to the art, to encode therapeutic or commercially useful deoxyribonucleic acid (DNA) or ribonucleic acid (RNA) sequences, including through the use of CRISPR/CAS9 (see CRISPR-Cas: A Laboratory Manual (2016), edited by Doudna and Mali or other methods included in the category of site-specific genetic modification (see Genome Editing Using Engineered Nucleases and Their Use in Genomic Screening (2017), edited by Costa et al.).

In accordance with an aspect of the present invention, there is provided a method of producing a desired, developmentally activated cell population (e.g. totipotent, pluripotent, pluripotent-like, neuronal, muscle, etc.) from the selected cells. This patent application covers “developmental activation” of any nucleated through application of an Electrical field (see Gagne et al., 1991; Saito et al., 2001; Yuan, 2008; Huang et al., 2007; Xia and Zhang, 2007; Cemazar and Sersa 2007; Isaka and Imai, 2007; Luxembourg et al., 2007; Van Tendeloos, 2007; Takahashi, 2007; etc.), liposomes, nanocapsules, nanovaults, etc. in the absence of nucleic acid or protein reprogramming factors (see Goldberg et al., 2007; Li et al., 2007), and/or another approach avoiding viral integration or other random alteration of the cell's genome, as such means increase safety and efficiency. Excluded, of course, from the category of “random alteration” are approaches involving gene-targeting and site-directed methods (e.g. CRISPR/CAS9) designed to introduce or remove DNA at specific locations in the genome; and the use of CRISPR/CAS9 to practice the invention is covered by the present invention. It follows that the present invention likewise covers the use of CRISPR/CAS9 (especially protein CAS9) in combination with the application of an Electrical field to produce modified cells of various cell types, according to the methods taught herein.

In some embodiments, the modification will represent a “genetic correction” when the gene modified is a disease associated gene (see below for a list of such genes). Likewise, this patent application covers the developmental activation, of any nucleated cell in the using electroporation, liposomes, nanocapsules, nanovaults, etc., as such means increase safety.

In a separate preferred embodiment, nucleic acid(s) or protein(s) corresponding to ZCAN4, PPP1R14A, DNMT3B, ZFP42, EPCAM, OCT4, CXCR4, SOX2, PRR+ NUMB, NOTCH NANOG, HOXB4, and/or a gene with LIF activity are the only adjunct nucleic acid(s) or protein(s) overexpressed and/or introduced to produce dividing, self-renewing, multipotent, pluripotent, totipotent, cells from the selected cells. Similarly, it should be understood that the methods described herein for initiating differentiation are applicable to any induced or non-induced multipotent, pluripotent, totipotent, or self-renewing stem cells, other progenitor cells, or other somatic cells, not only those obtained in the manner described herein.

It is to be understood that any combination of nucleic acid or protein sequences (and/or their corresponding proteins) described herein can be modified by excluding nucleic acid sequences (and/or their corresponding proteins) or proteins corresponding to Numb and/or Numblike so long as the desired cell population is achieved.

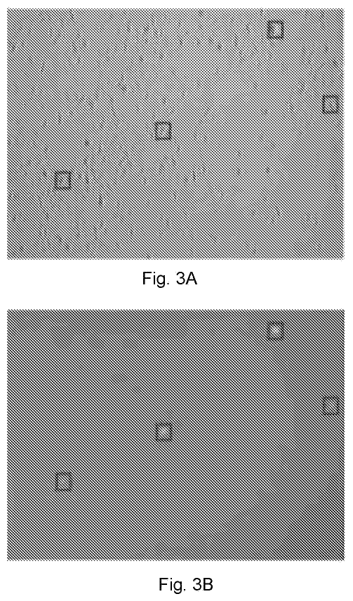

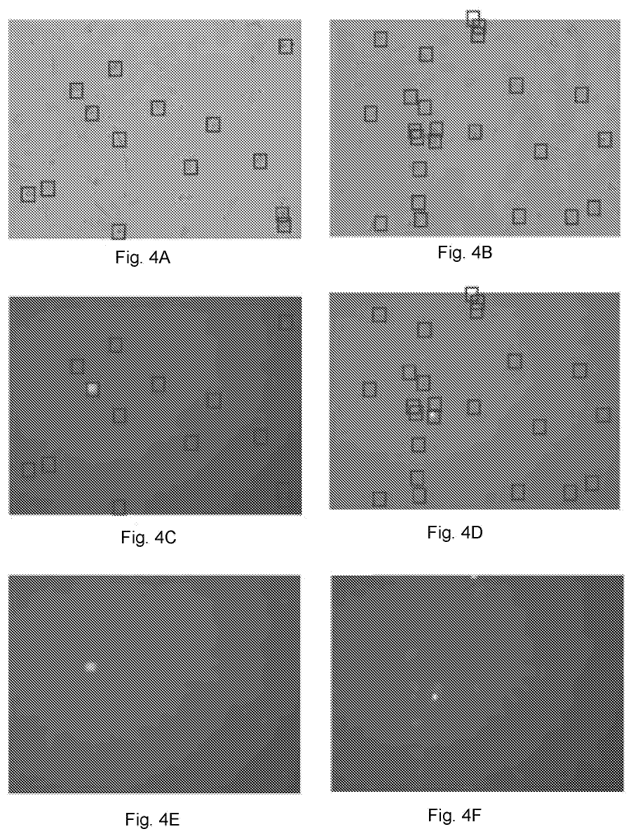

In a preferred embodiment, the selected cells and/or their progeny are cells that have been genetically-modified. In a further preferred embodiment transient transfection is accomplished using viral vectors that do not integrate into the host genome. Non-integrating and episomal viral vectors are well known to the art and include 2^{nd} and 3^{rd} generation, integrase-deficient, non-integrating lentiviral vectors, including 3^{rd} generation lentivectors taught herein. Such integrase-deficient vectors can be readily introduced using a variety of standard transfection techniques (e.g. electroporation, chemically mediated transfection, fusogenic or non-fusogenic liposomes, lipofectamine, nanocapsules, nanovaults, etc.)—methods which allow high capacity integrase-deficient lentiviral vectors to be utilized without genomic integration and random alteration of the genome (see FIG. 3D).

This patent application also covers the developmental activation of any nucleated cell utilizing nucleic acid or protein electroporation to a differentiating cell type (for example methods see Gagne et al., 1991; Saito et al., 2001; Yuan, 2008; Huang et al., 2007; Xia and Zhang, 2007; Cemazar and Sersa 2007; Isaka and Imai, 2007; Luxembourg et al., 2007; Van Tendeloos, 2007; Takahashi, 2007; etc.) electroporation, liposomes, nanocapsules, nanovaults, and/or another approach avoiding viral integration or other random alteration of the cell's genome as such means increase safety and efficiency.

In another preferred embodiment, selected cells are transfected with (or overexpress) of one or more adjunct sequences (or their corresponding proteins) encoding ZCAN4, PPP1R14A, DNMT3B, ZFP42, EPCAM, OCT4, CXCR4, SOX2, PRR+ NUMB, NOTCH NANOG, HOXB4, and/or a gene with LIF activity and/or one or more Transcription Factor, Kinase or Small RNA enriched in developmentally-activated human and/or mouse cells (see lists of such enriched small RNAs, transcription factors, and kinases below).

In another preferred embodiment, cells treated with electroporation to produce factor free, developmentally-activated are further cultured in a cell culture promoting an optimal growth rate, such as described above, and that includes EGF, bFGF, oncostatin, LIF (e.g. Du and Shi, 1996), steel factor, IL-11, cardiotrophin-1, IL-6, hyper-IL-6, CNTF, soluble gp130 and/or one or more small molecules. In a preferred embodiment, the cells are grown in a serum free growth medium.

### Assessment of Totipotency, Potency and Differentiation

Totipotency can be assessed to cells demonstrating competence to generate a complete embryo. Additionally, zscan4 is a marker of totipotent cells and populations containing totipotent cells can be assessed using antibodies against zscan4 and/or PCR, and transcriptome based analyses.

Pluripotency and multipotency can be assessed by any means known to the art including 1) transplantation, 2) culture under conditions promoting embryoid body formation, 3) injection of cells into animal blastocyst stage embryos with subsequent development, and 4) RNA expression assays (e.g. RT-PCR and microarray based analyses) for gene expression associated with differentiation, multipotency, pluripotency, etc. (see Guan et al., 2006), 5) colony-formation, as well as by ES-like morphology. One approach disclosed herein for detecting pluripotency in selected cells and/or their progeny involves transfection with (or overexpression of) a reporter construct comprising the Nanog promoter operably linked to a fluorescent protein gene. This allows identification and enrichment of Nanog expressing cells using Fluorescence Activated Cell Sorting (FACS), etc. In a preferred embodiment, endogenous cells are transfected in vivo with genetic vectors encoding the sequences (or corresponding proteins) named herein to transiently promote renewed or increased cell proliferation. This approach can also be utilized clinically in the setting of hypoplastic tissues, disorders where stem/progenitor cells are abnormally depleted, and other disorders where the approach can be shown to be beneficial.

### Achieving Differentiating Cell Populations

In order to achieve a variety of differentiating cell populations capable of further environmentally-regulated differentiation in vivo, developmentally activated cells and/or other selected cells are presented for introduction or overexpression of nucleic acid sequences (or proteins) corresponding to transcription factors and other cell fate determinants normally expressed in the desired differentiating cells.

In most instances, the cells are then cultured in the presence of agents(s) promoting differentiation of the selected cells and/or their progeny into a desired cell population. The presence of the agents(s) provides for growth or proliferation at a rate that is less than the optimal (or expansion) growth rate, and favors differentiation of the cells into a desired cell population. The agents and precise culture conditions are selected according to the desired cell population as described below.

In separate embodiments, for the purpose of developmentally activating cell populations, it may be desirable to perform only electroporation and the incubation steps above. Appropriate concentrations of LIF and steel factor for stem/progenitor cell propagation/proliferation as well as other cell culture conditions have been described previously (e.g. U.S. Pat. Nos. 6,432,711 and 5,453,357 herein incorporated by reference). Other appropriate protocols and reference cytokine concentrations have been taught by Koshimizu et al., 1996; Keller et al., 1996; Piquet-Pellorce, 1994; Rose et al., 1994; Park and Han, 2000; Guan et al., 2006; Dykstra et al., 2006).

### Achieving Differentiated or Differentiating Cell Populations

When the desired cell population is a neural cell population, the cells are cultured under conditions that promote growth at a rate which is less than the optimal rate and in the presence of agent(s) promoting differentiation of the cells into neural cells. Conditions promoting differentiation into neurons have been described in numerous publications including (Benninger et al., 2003; Chung et al. 2005; Harkany et al., 2004; Ikeda et al., 2004; Ikeda et al., 2005; Wernig et al., 2002; and Wernig et al., 2004). Furthermore, combining retinoic acid exposure with the presence of additional cytokines favors specific neuronal cell type differentiation in vitro (e.g. Soundararajan et al., 2006; Soundararajan et al., 2007; U.S. Pat. No. 6,432,711). In a preferred embodiment, in vitro differentiation of neurons or neural cells occurs in the presence of 50 ng/mL nerve growth factor (NGF).

In another preferred embodiment, when a neuronal or neural cell population is the desired population, the cells are incubated in cell culture in a cell culture medium promoting differentiation, such as described above and that includes one or more of the following agents: retinoic acid, NT3, NGF, glial cell-line derived growth factor (GDNF), and interferon gamma (IFN-gamma).

When the desired cell population is a muscle population, the selected cells may be cultured in the presence of an agent promoting differentiation of the cells into muscle cells and growth at a rate less than the optimal rate. Conditions promoting differentiation into muscle cells have also been described previously (Nakamura et al., 2003; Pal and Khanna, 2005; Pipes et al., 2005; Albilez et al., 2006; Pal and Khanna, 2007; Behfar et al., 2007; U.S. Pat. No. 6,432,711). Furthermore, exposure of selected cells and/or their progeny to hexamethylene bis-acrylamide or dimethylsulfoxide in the presence of additional cytokines favors the initiation of muscle type differentiation in vitro.

When the desired cell population is a hematopoietic cell population, the differentiation medium may include specific agents at concentrations promoting differentiation into hematopoietic progenitor cells (e.g. vascular endothelial growth factor (VEGF), thrombopoietin, etc. (e.g. Ohmizono, 1997; Wang et al., 2005; Srivastava et al., 2007; Gupta et al., 2007) or differentiated hematopoietic cell types (according to methods known to the art for providing differentiated hematopoietic cell types from undifferentiated or pluripotent cells).

When the desired cell population is a germ cell population, the differentiation medium may include specific agents at concentrations promoting differentiation into germ cells (e.g. Nayernia et al. 2006a, 2006b).

When the desired cell population is a germ cell population, the differentiation medium includes specific agents at concentrations promoting differentiation into germ cells (e.g. Nayernia et al. 2006a, 2006b).

When the desired cell population is an endoderm and pancreatic islet cell population, the differentiation media may include specific agents at concentrations promoting differentiation into endoderm and pancreatic islet cells (e.g. Xu et al., 2006; Denner et al., 2007; Shim et al., 2007; Jiang et al., 2007).

In another preferred embodiment, differentiation of selected cells and/or their progeny may occur in the differentiation medium in the absence of transfection with (or overexpression of) numblike, short Numb isoforms (or their corresponding proteins), although the differentiation medium may be unchanged.

### Factors Promoting Differentiation

Simultaneous transfection with (or overexpression of) any subset of these distinct transgene sequences (and/or their corresponding proteins) listed above can be accomplished by any means known to the art including electroporation, the use of multiple genetic vectors, serial transfection as well as selection based on distinct marker proteins and/or antibiotic resistance.

In a preferred embodiment, a differentiated cell population is desired and introduction or overexpression of sequences encoding short numb isoforms and/or numblike and/or their corresponding proteins is accompanied or replaced by introduction or overexpression of other nucleic acids and/or proteins, e.g. one or more of a. Nurr1, REN, Neurogenin1, Neurogenin2, Neurogenin3, Mash 1, Phox2b, Phox2a, dHand, Gata3, Shh, FGF8, Lmx1a, Lmx1b, Nkx2.2, Pet1, Lbx1, Ptx-3, Pitx2, Dlx1, Dlx2, Dlx5, Rnx, miR-124 and miR-128, when neurons are the desired cell population; b. Mash1, Ngn2, Nurr1, Lmx1a, Lmx1b, and/or Ptx-3, when dopaminergic neurons are the desired population; c. Mash1, Phox2b, Lmx1b, Nkx2.2, Gata2, Gata3 and/or Pet1, when serotonergic neurons are the desired cell population; d. MASH1, Phox2a and/or REST4 when cholinergic neurons are the desired cell population; e. MASH1, Phox2a, PITX2, Dlx2, Dlx5, REST4 and/or anti-Hes1 synthetic oligonucleotides, followed, optionally, by culture in media supplemented with LIF, Neurotrophin 3 (NT3), and/or nerve growth factor (NGF), when Gabaergic neurons are the desired cell population; and f. Mash1, dHand, Phox2a, Phox2b, Gata2 and/or Gata3, when noradrenergic neurons are the desired cell population; g. Gata 4, Gata 5, Gata 6, myocardin, Hand2, Mef2c, and Tbx5, miR-1-1 and miR-1-2 and/or by cell culture in a cell culture medium promoting differentiation into cardiomycytes (He et al., 2003; Guan et al., 2007; etc.), or that includes specific agents at concentrations promoting cardiac cell differentiation (e.g. 0.75%4% dimethyl sulfoxide (DMSO), 20% normal bovine serum (NBS), 10(−7) mM retinoic acid (RA) and 20% cardiomyocytes conditioned medium (Hua et al., 2006), when the desired cell population is a cardiac cell population; h. muscle type specific bHLH-encoding sequences (or their corresponding proteins), MyoD, Myogenin, Myf5, Myf6, Gata 4, Gata 5, and Gata 6, Mef2, Myocardin, Ifrd1 and/or other muscle transcription factors and small RNAs, when a muscle cell population is the desired population; i Myocardin, when smooth muscle is the desired cell population; j. MyoD, myogenin, miR-1, miR-1-1, miR-1-2, miR-206, miR-26a, miR-133, miR-133a-1 and miR-133a-2. when skeletal muscle is the desired cell population; OLIG1, OLIG2, and Zfp488, when oligodendrocyte cell population is the desired cell population, k. FIGLA, FIG alpha, DAZL, STRA8, FOXL2, OOGENESIN1, OOGENESIN2, OOGENESIN3, OOGENESIN4, SYCP2, SYCP3, SPO11, REC8, DMC1, MOS, STAG3, CCNB1, FOXO1, FOXO3, SOHLH1, SOHLH2, NOBOX, OBOX1, OBOX2, OBOX3, OBOX4, OBOX6, LHX8, LHX9, OOG1, SP1, ZFP38, TRF2, TB2/TRF3, TAF4B, TAF7L, TAF7l, TIA1, PHTF1, TNP2, HILS1, DAZL, BMP15, PTTG3, AURKC, OTX2, SOX15, SOX30, FOXR1, ALF, OCT4, DPPA3/STELLA, ZFP38, RPS6KA3, HINFP, NPAT, SP1, SP3, HOXA1, HOXA7, HEX, YP30, ZP1, ZP2, ZP3, SFE1, SFE9, OPO, PLN, RDV, GLD1, MMU-MiR351, MMU-MiR615, MMU-MiR592, MMU-MiR882, MMU-MiR185, MMU-MiR491, MMU-MiR326, MMU-MiR330, MMU-MiR351, when a germ cell population is the desired population; l. SYCP2, SYCP3, SPO11, REC8, DMC1, MOS, STAG3, OCT4, ALF, RPS6KA3, HINFP, SP1, SP3, TAF7l, TIA1, PHTF1, TNP2, HILS1, CLGN, TEKT1, FSCN3, DNAHC8, LDHC, ADAM3, OAZ3, AKAP3, MMU-MiR351, MMU-MiR615, MMU-MiR592, MMU-MiR882, and MMU-MiR185, when a sperm or spermatocyte population is the desired population; m. MOS, CCNB1, OCT4, FIG alpha, FIGL alpha, ALF, SOHLH1, SOHLH2, LHX8, LHX9, OOG1, FIG alpha, SP1, LHX3, LHX9, TBP2/TRF3, DAZL, BMP15, GDF9, PTTG3, AURKC, OTX2, SOX15, SOX30, FOXR1, NOBOX, OBOX1, OBOX2, OBOX3, OBOX6, OOGENESIN1, OOGENESIN2, OOGENESIN3, OOGENESIN4, YP30, ZP1, ZP2, ZP3, SFE1, SFE9, OPO, PLN RDV, GLD1, DAZL, STRA8, MMU-MiR615, MMU-MiR491, MMU-MiR326, MMU-MiR330, MiR212 and MMU-MiR351, when an oocyte population is the desired population; n.

Foxa2, Sox17, HLXB9, Ngn3, Mafa and/or Pdx1, when an endoderm or pancreatic islet population is the desired population; o. hepatic nuclear factor (HNF)-1, HNF-3, HNF-4, HNF-6 and creb-binding protein, when hepatocytes are the desired population, p. Runx1/AML1 (the Runx1/AML1a isoform is introduced when engraftment is desired and the b isoform when differentiation is desired, NOV (CCN3), miR-128, miR-181, miR-16, miR-103 and miR-107, and/or cell culture in the presence of colony stimulating factors specific for the desired cell populations when hematopoietic cells are the desired cells; q. miR-150 when a T lymphoid cell population, the introduced or overexpressed sequences may include miR-150; r. miR-181, miR-155, miR-24, miR-17, miR-16, miR-103 and miR-107, when the desired hematopoietic cell is a B lymphoid cell population; s. miR-150, miR-155, miR-221, miR-222, miR-451, miR-16 and miR-24, when the desired hematopoietic cell is an erythroid cell population; t. miR-17-5p, miR-20a, miR-106a, miR-16, miR-103 and miR-107, when a monocyte cell population is the desired population; u. miRNA-155, miR-24, miR-17, miR-223, miR-16, miR-103 and miR-107 when a granulocyte cell population is the desired population; v. miR-155, miR-24, and miR-17, when the desired cell is a megakaryocyte cell population; w. Sox9, CREB binding protein, Gata6, Run2, and TGF beta, when chondrocytes are the desired population; x. Runx2, mir-125b, miR26a, when bone cells (especially osteoblasts) are the desired population; and y. miR-203 when a keratinocyte population is the desired cell population.

In a preferred embodiment, the genetic vectors encoding the long Numb isoforms (such as those described herein) are introduced transiently or under the control of a regulable promoter, into endogenous cells in vivo in order to cause those cells proliferate transiently.

Alternatively, proteins and peptides corresponding to Numb isoforms, Notch, OCT3/4, SOX2, and/or other DNA sequences (and/or their corresponding proteins) listed herein may be applied in analogous fashion to selected cells and/or their progeny via electroporation (e.g. Koken et al., 1994; Ritchie and Gilroy, 1998), using nano particles, cationic lipids, fusogenic liposomes (e.g. Yoshikawa et al., 2005; 2007), etc. in lieu of, or in combination with genetic transfection. Generally, electroporation allows for high transfection efficiency (and efficient production of the desired cells) without genomic integration of the transgene and is therefore associated with increased safety. The DNA or RNA encoding protein(s) or polypeptide(s) promoting proliferation, multipotency, pluripotency or differentiation of the selected cells may be isolated in accordance with standard genetic engineering techniques (for example, by isolating such DNA from a cDNA library of the specific cell line) and placing it into an appropriate expression vector, which then is transfected into the selected cells.

In a preferred embodiment, endogenous cells (e.g. ependymal zone cells of the central nervous system) are transfected in vivo with genetic vectors encoding either the shortest numb isoform or the numblike protein(s) alone or in conjunction with other transgenes (and/or their corresponding proteins) named herein, in order to transiently or permanently promote renewed or increased differentiation (especially neuronal differentiation) and migration of progenitor/ependymal cells in the central nervous system). This renewal or increase is measured in terms of the number of cells showing new-onset expression of markers associated with differentiation. This may be accomplished by introduction of the genetic vectors into the organ system using methods suitable for that purpose (see examples).

In a preferred embodiment, endogenous cells (e.g. ependymal zone cells of the central nervous system) are transfected in vivo with genetic vectors encoding the long numb isoform(s) and/or other transgenes (and/or their corresponding proteins) named herein, in order to transiently promote renewed or increased stem cell proliferation (with subsequent differentiation of progeny cells). This renewal or increase is measured in terms of the number of cells showing new-onset expression of markers associated with dividing progenitors. This may be accomplished by introduction of the genetic vectors into the organ system using methods suitable for that purpose (see examples).

Likewise, this approach is also be suitable for inducing renewed or increased differentiation from other stem cell populations in other tissues (such as the skin, etc). This approach can be utilized, for example, clinically in the setting of central nervous system injury, disorders of other tissues where normal differentiation or migration are inadequate, dysplastic disorders and other disorders where the approach is beneficial.

In a preferred embodiment, in order to produce developmental activation, nucleic acid(s) or protein(s) corresponding to a single gene, or portion thereof, (particularly those named herein, discovered according to methods described herein, discovered according to other published methods; and/or known to be capable of initiating the desired manner of differentiation) are a. the only nucleic acid(s) or protein(s) overexpressed and/or introduced to initiate differentiation in the selected cells; b. the method utilized is electroporation, liposomes, nanocapsules, nanovaults, and/or another approach avoiding retroviral/lentiviral integration or other random alteration of the cell's genome; c. other nucleic acid(s), protein(s) or other transfectants can be utilized in concert with the nucleic acid(s) or protein(s) corresponding to a single gene, or portion thereof, (particularly those named herein, discovered according to methods described herein; d. the method utilized is electroporation, liposomes, nanocapsules, nanovaults, and/or another approach avoiding retroviral/lentiviral integration or other random alteration of the cell's genome.

It is to be understood that any combination of nucleic acid or protein sequences (and/or their corresponding proteins) described herein can be modified by excluding those corresponding to Numb and/or Numblike so long as the desired cell population or behavior is achieved.

Similarly, it should be understood that the methods described herein (or elsewhere) for initiating differentiation are applicable to any induced or non-induced multipotent, pluripotent, totipotent, or self-renewing stem cells, or other selected cells, not only those obtained in the manner described herein.

### Sources of Selected Cells

The population of selected cells may derive from various stem cells, progenitor cells and somatic cells. However somatic cells lacking nuclei (e.g. mature, human red blood cells) are specifically excluded. Selected stem cells may be derived from existing cell lines or isolated from stored, banked, or cryopreserved sources. Typical sources of stem cells include bone marrow, peripheral blood, placental blood, amniotic fluid (e.g. De Coppi et al., 2007), umbilical cord blood (e.g. Zhao, et al., 2006; Tian et al., 2007), adipose tissue (e.g. Gimble et al., 2007; Ma et al., 2007), non-human embryos, and others. Circulating leukocytes and other non-stem cells may likewise be selected and subjected to the same culture conditions as described above effective that they acquire multipotency, pluripotency, totipotency and/or self-renewal as a result. Examples of other accessible somatic cells useful in this invention include lymphocytes and epithelial (e.g. buccal cheek and urine) cells. Isolation and collection of cells selected for use within the present invention may be performed by any method known to the art.

In embodiments involving animals, stem cells isolated from prostate, testis, embryonic brain, and intestine are also disclosed as being preferred sources of selected cells.

In a preferred embodiment, the selected cells and/or their progeny are used in conjunction with tissue engineering and organ production methods (e.g. Tissue Engineering A, B and C, edited by Mikos and Fisher). It is disclosed herein that the cells provided by the methods described herein (or similar methods) be utilized in conjunction with techniques aimed at the production of organs and/or tissues (e.g. Boland et al., 2006. Xu et al., 2006; Campbell and Weiss, 2007). Such utilization is specifically covered by the present invention. For instance, cells produced or treated according to the methods described herein (or other published methods) may be grown in association with three-dimensional or two-dimensional format and/or scaffoldings engineered to replicate normal tissue structure and/or organ structures (e.g. Yarlagada et al., 2005; Kim et al, 1998; WO/2003/070084; EP1482871; WO03070084; U.S. Pat. Nos. 2,395,698; 7,297,540; 6,995,013; 6,800,753; Isenberg et al., 2006).

Similarly, scaffoldings to be occupied by the pluripotent, multipotent, and/or differentiating cells may be derived from cadaveric organ(s) or tissue(s) after the cadaveric organs or tissues (e.g. bone, target tissue, organ or cavity, kidney, liver, lung, etc.) may be treated in such away that the host immune cells resident in that tissue, and other undesirable or ancillary host cells, are eliminated (e.g. by ionizing radiation, sterilization (e.g. Mroz et al., 2006), and/or various methods of decellularization (U.S. Pat. Nos. 6,734,018; 6,962,814; 6,479,064; 6,376,244; 5,032,508; 4,902,508; 4,956,178; 5,281,422, 5,554,389; 6,099,567; and 6,206,931; 4,361,552 and U.S. Pat. Nos. 6,576,618; 6,753,181; U.S. application Ser. No. 11/162,715; WO/2001/048153; WO/2002/024244; WO003002165; WO/2001/049210; WO/2007/025233; European Patents EP1482871; EP1246903; EP1244396; EP0987998; EP1244396; EP1333870; Rieder et al., 2004; Ott et al., 2008; Taylor et al., 1998; Choudhury, 2018).

Likewise, it is anticipated that the pluripotent, multipotent, and/or differentiating cells of the present invention may be used in applications utilizing 3D printing (Beheshtizadeh et al., 2020; Wragg 2019; Yan, 2018) inkjet-style printing for tissue engineering (e.g. Boland et al., 2006. Xu et al., 2006; Campbell et al., 2007; Gungor-Oskerim, 2018). Therefore, such use of the cells produced or treated according to the methods described herein is covered.

In another preferred embodiment, the selected cells and/or their progeny are cultured in hanging drops. In accordance with another aspect of the present invention, selected cells may be modified genetically beforehand.

### Screening Cell Populations

In one embodiment, the methods of this invention comprise screening cells from cell lines, donor sources, umbilical cord blood, and autologous or donor bone marrow, blood, spermatogonia, primordial germ cells, buccal cheek cells, or any other cell source effective in the current invention. Selected cells can be screened to confirm successful introduction/overexpression of nucleic acids and/or proteins by any method known to the art (Guan et al., 2006; U.S. Pat. No. 6,432,711). In some embodiments, the cells are screened using standard PCR and nucleic acid hybridization-based methods or using rapid typing methods. In preferred embodiments, the cells are screened according to expression of reporter genes. In some embodiments, cells are screened by expression of a marker gene encoded by the transgene expressing vector(s) such as an antibiotic resistance gene or a fluorescent protein (e.g. GFP) gene.

Screening for Therapeutic Vectors and Beneficial Sequences (or their Corresponding Proteins)

Cells can be screened for the presence of beneficial sequence(s) and therapeutic vector(s) using any method(s) known to the art for detection of specific sequences (or their corresponding proteins). Each cell sample can be screened for a variety of sequences (and/or their corresponding proteins). Alternatively, multiple samples can be screened simultaneously.

Cell differentiation may be monitored by several means: including (i) morphological assessment, (ii) utilizing reverse transcriptase polymerase chain reaction (RT-PCR), Northern blot, or microarray techniques to monitor changes in gene expression, (iii) assaying cellular expression of specific markers such as beta tubulin III (for neurons) etc. (Ozawa, et al., 1985). In some embodiments, the cells are screened for successful initiation of differentiation using FACS sorting based on cell type specific markers or transgenic marker expression (e.g. antibiotic resistance or fluorescent protein expression) under the control of cell type specific promoters such as the myosin promoter in muscle cells; the human cardiac α-actin promoter in cardiomyocytes; the insulin promoter in insulin producing cells; the neuronal-specific enolase (NSE) promoter for neuronal differentiation, or neurotransmitter related promoters such as the tyrosine hydroxylase promoter in dopaminergic neurons; etc.).

In some embodiments, the cells are screened using standard PCR and nucleic acid hybridization-based methods. In a particularly preferred embodiment, the cells are screened using rapid typing methods.

### Screening for Human Leukocyte Antigen (HLA) Type

In certain embodiments, the selected cells are selected with respect to compatible HLA typing. The HLA genotype can be determined by any means known to those of skill in the art.

The cells used for screening may consist of cells taken directly from a donor, or from cell lines established from donor cells, or other practicable cell sources. The cells can be screened for beneficial sequence(s), and/or therapeutic vector(s) and HLA type at once, or separately. Those cells successfully transfected with a beneficial sequence and showing an appropriate HLA genotype can be prepared for transplantation to a patient. In certain embodiments, the transfected cells are transplanted without HLA typing. In other embodiments, the cells are HLA typed for compatibility.

### Screening for Agents Promoting a Cellular Phenotype.

The present invention also provides for a methods of screening proteins and agents for their ability to produce developmental activation of the selected cells and/or their progeny into desired cell populations. Briefly, vectors encoding complementary DNAs (cDNAs) from appropriate cDNA libraries are transfected into the selected cells/and or their progeny. Once a specific cDNA that induces differentiation or other phenotypic change is identified, such cDNA then may be isolated and cloned into an appropriate expression vector for protein production in appropriate cells (e.g. COS cells) in vitro. Later the protein containing supernatant can be applied to the selected cell cultures to determine if any secreted proteins from such cells induce differentiation Alternatively, candidate agents can be applied to the selected cell cultures to determine if any of the candidates induce developmental activation.

The present invention also provides for methods of screening nucleic acids for their ability to induce multipotentiality, pluripotency, and/or self-renewal, or to initiate differentiation of selected cells and/or their progeny. In these methods, vectors encoding selected cDNAs (or cDNAs from appropriate cDNA libraries, or other sequences (or their corresponding proteins)) are introduced into the selected cells/and or their progeny using electroporation, nanocapsules, nanovaults, liposomes, retroviruses, lentiviruses, and/or any other practicable means of transfection. Once a specific cDNA that induces a phenotypic change, multipotentiality, pluripotency, and/or self-renewal, is identified, such cDNA then may be isolated and cloned into an appropriate expression vector. Assays for determining such changes include those described elsewhere herein.

Likewise the protein corresponding to the identified cDNA may be produced in appropriate cells (e.g. COS cells) in vitro to determine whether the protein containing supernatant can be applied to the selected cell cultures and induce the desired changes.

Finally, proteins may be introduced into the selected cells/and or their progeny using electroporation, nanocapsules, nanovaults, liposomes, retroviruses, lentiviruses, and/or any other practicable means of transfection, and the resulting cells assessed as described herein for multipotentiality, pluripotentiality, self-renewal or the initiation of differentiation.

Transplantation of Cells into Patients or Subjects

After screening, selected cells and/or their progeny may be cryopreserved, maintained as cell lines in culture, or may be administered to the patient. Selected cells can be cryopreserved or maintained in culture by any means known to the art and preserved for future transplantation procedures.

Preferably, the cells to be screened are obtained from accessible sources allowing easy collection.

With regard to producing HIV resistant cells: targeted somatic cells and stem cells of this invention can be of any type capable of differentiating into cells that can be infected by HIV, that can sustain the transcription and/or replication of HIV, that can alter the HIV immune response, or that can retard progression to AIDS. Such stem cells include, but are not limited to, developmentally-activated cells, pluripotent cells derived from spermatogonia, primordial germ cells, hematopoietic stem cells, peripheral blood cells, placental blood cells, amniotic fluid cells, umbilical cord blood cells, buccal cheek cells, adipose tissue cells (including stem cells derived from those tissues) induced multipotent cells, induced pluripotent cells, etc., non-human embryos, and/or any other cell type that can form blood and immune cells, HIV target cells, and other cells.

Therapeutic vector(s) express “beneficial sequence(s)” intended to render transfected or infected cells less capable of sustaining HIV replication and transcription. The genetic vector expressing “beneficial sequence(s)” as well as any virus derived from such genetic vector, are herein termed “therapeutic vector”.

After screening, cells transfected with the desired therapeutic vector(s) and expressing beneficial sequence (with or without compatible HLA genotype) may be expanded ex vivo (in vitro) using standard methods to culture dividing cells and maintained as stable cell lines (U.S. Pat. Nos. 6,432,711 and 5,453,357 herein incorporated by reference). Alternatively, these cells can be administered to the patient and expanded in vivo.

Selected cells can be cryopreserved by any means known to the art and preserved for future transplantation procedures.

Transplantation of Desirable Cell Populations into Patients or Subjects

In certain embodiments, cell populations are enriched for stem cells prior to transplantation. Various methods to select for stem cells are well known in the art. For example, cell samples can be enriched by fluorescently labeled monoclonal antibodies recognizing cell-surface markers of undifferentiated hematopoietic stem cells (e.g., CD34, CD59, Thyl, CD38 low, C-kit low, lin-minus) for sorting via fluorescence-activated cell sorting (FACS). In other embodiments, a sample of the selected cells is transplanted, without enrichment. In some embodiments, the endogenous stem cells of the bone marrow are eliminated or reduced prior to transplantation of the therapeutic stem cells. Therapeutic stem cells are defined as those stem cells containing beneficial sequence(s) or therapeutic vector(s).

In some embodiments, the transplantation process may involve the following phases: (1) conditioning, (2) stem cell infusion, (3) neutropenic phase, (4) engraftment phase, and (5) post-engraftment period. In some embodiments, the endogenous stem cells that normally produce the desired cells (e.g. bone marrow stem cells) are eliminated or reduced prior to transplantation. Chemotherapy, radiation, etc. and/or methods analogous to those described in U.S. Pat. No. 6,217,867 may be used to condition the bone marrow for appropriate engraftment of the transplant. Finally, therapeutic stem cells may be transplanted into the patient using any method known to the art.

### Sample Transgene Encoding Vectors

In one embodiment transfection with (or overexpression of) nucleic acid sequence(s) encoding transgenes is accomplished via viral transfection. The term “transgene encoding vector(s)” refers to the vectors incorporating the nucleic acid sequence(s) encoding transgenes named herein, especially encoding one or more transgenes named herein, as well as any additional sequences, synthetic oligonucleotides, etc, and any associated viral supernatant incorporating those vector sequences.

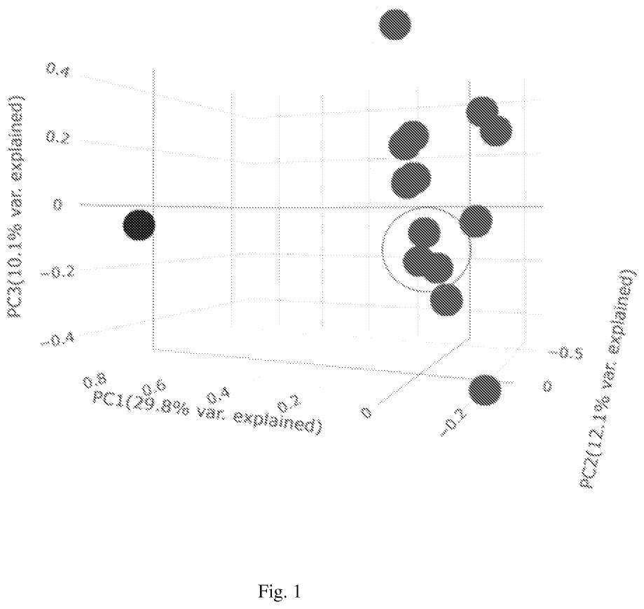

In one embodiment, the transgene encoding vector(s) comprise two or more transgenes named herein for producing developmental activation. See FIG. 1D.

The transgene encoding vector(s) may comprise an expression vector. Appropriate expression vectors are those that may be employed for transfecting DNA or RNA into eukaryotic cells. Such vectors include, but are not limited to, prokaryotic vectors such as, for example, bacterial vectors; eukaryotic vectors, such as, for example, yeast vectors and fungal vectors; and viral vectors, such as, but not limited to adenoviral (Lin et al., 2007) vectors, adeno-associated viral vectors, and retroviral vectors. Examples of retroviral vectors which may be employed include, but are not limited to, those derived from Moloney Murine Leukemia Virus, Moloney Murine Sarcoma Virus, and Rous Sarcoma Virus, FIV, HIV, SIV and hybrid vectors, including the episomal, integrase-deficient, non-integrating, 3^{rd} generation engineered lentiviral vectors_(see FIG. 3D),), described herein and/or described in references cited herein. Such vectors can be introduced safely without genomic integration or random alteration of the genome using electroporation and other methods taught herein.

It is disclosed that the transgene encoding vector(s) may be used to transfect cells in vitro and/or in vivo. Transfection can be carried out by any means known to the art, especially through virus produced from viral packaging cells. Such virus may be encapsidated so as to be capable of infecting a variety of cell types. Nevertheless, any encapsidation technique allowing infection of selected cell types and/or their progeny is practicable within the context of the present invention.

### Design of Human Immunodeficiency Virus (HIV) Gene Therapy Vector(s)

The present invention teaches vectors as an antiviral strategy. The approach is illustrated with respect to an immunodeficiency virus infection. The “therapeutic vector(s)” may incorporate an expression vector. Appropriate expression vectors are those that may be employed for transfecting DNA or RNA into eukaryotic cells. Such vectors include, but are not limited to, prokaryotic vectors such as, for example, bacterial vectors; eukaryotic vectors, such as, for example, yeast vectors and fungal vectors; and viral vectors, such as, but not limited to adenoviral (Lin et al., 2007) vectors, adeno-associated viral vectors, and retroviral vectors. Examples of retroviral vectors which may be employed include, but are not limited to, those derived from Moloney Murine Leukemia Virus, Moloney Murine Sarcoma Virus, and Rous Sarcoma Virus, feline immunodeficiency virus (FIV), HIV, simian immunodeficiency virus (SIV) and hybrid vectors, including the replication incompetent, integrase-deficient, 3^{rd} generation, engineered, episomal, non-integrating lentiviral vectors (see FIG. 3D),), described herein and/or described in references cited herein. Such vectors can be introduced safely without genomic integration or random alteration of the genome using electroporation and other methods taught herein.

It is disclosed herein that the therapeutic vector(s) may be used to transfect target cells in vitro and/or in vivo. Transfection can be carried out by any means known to the art, especially through virus produced from viral packaging cells. Such virus may be encapsidated so as to be capable of infecting a cell, e.g. a developmentally-activated cell, a CD34+ cell and/or CD4+ cell. However, in some instances, other cell types are transfected by means not involving the CD4 or CD34 proteins. Nevertheless, any encapsidation technique allowing infection of such cell types may therefore be included in the disclosure of the present invention.

Pseudotyping with different envelope proteins expands the range of host cells transducible by viral vectors and therapeutic vectors and allows the virus to be concentrated to high titers, especially when pseudotyped with the vesicular stomatitis virus envelope glycoprotein (VSV-G) (Li et al., 1998; Reiser et al., 2000).

### Vector Construction

Viral vectors utilized in this invention may be of various RNA and DNA virus types, including hybrid vectors. Vectors may, for instance, be third-generation lentiviral vectors which include only a very small fraction of the native genome (Zufferey et al., 1998). Production of transgene encoding vector(s) may also involve self-inactivating transfer vectors (Zufferey et al., 1998; Miyoshi et al., 1998) eliminating the production of full-length vector RNA after infection of target cells.

Viral vectors may be utilized which are replication-incompetent due to failure to express certain viral proteins necessary for replication. However, the possibility exists that helper virus may enable therapeutic virus replication. This likelihood can be reduced by the use of vectors that are self-inactivating, as well as replication-incompetent and non-integrating, as described in references cited herein.

In a preferred embodiment, transgene sequences (and/or their corresponding proteins) are driven by a ubiquitin promoter, U6 promoter, EF1alpha promoter, CMV promoter, regulable promoters and/or desired cell type specific promoters.

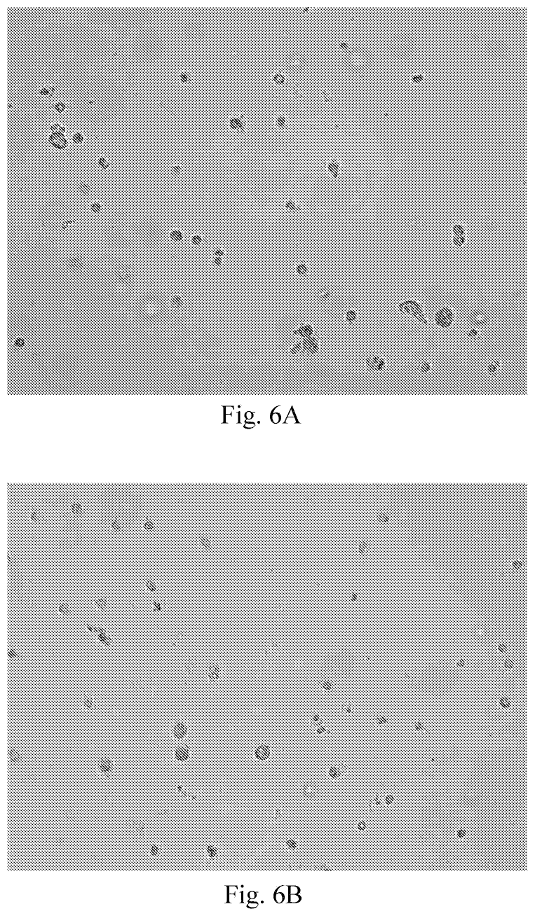

The lack of a functional integrase gene in these vectors renders them integrase-deficient and episomal, and is the consequence of inserting the EGFP expression cassette consisting of EGFP sequences and the CMV IE promoter into the region normally occupied the gag-pol genes (FIG. 6A of Reiser et al., 2000; see FIG. 1D herein).

When cell transduction is mediated by virus (i.e. viral particles) pol deletion interferes with reverse transcription of viral RNA to DNA. Accordingly, pol deletion results in a non-integrating or integration deficient lentivector. This is because “Integrase . . . is involved in the reverse transcription of HIV-1 RNA and nuclear import of the preintegration complex (PIC) (Gallay et al., 1997; Zhu et al., 2004; Philpott and Thraser, 2007).

However, when one skilled in the art chooses not to use infection and instead chooses electroporation to introduce DNA lentiviral vectors to the cells, as taught by the applicant, no reverse transcription is necessary; and because the pol gene is deleted, no integrase enzyme is present in the cell to support integration. It has been taught by Wanisch and Yanez-Munoz (2009) that,
“HIV-1 circles are considerably stable after infection, with progressive vector episome dilution due to cell division. Thus, the apparent decrease in circularized HIV-1 DNA after infection of CD4+ MT-2 or SupT1 T-cells is the result of ongoing cell division causing the dilution of nonreplicating viral episomes in the cell population. Episomes are stable in macrophages for at least 21 days (ref. 36), while a turnover of episomes has been observed in vivo in human peripheral blood mononuclear cells over the course of several weeks, suggesting that they can be slowly degraded.” (Wanisch and Yanez-Munoz, 2009).

Accordingly, those skilled in the art recognize that integration deficient lentivectors may persist episomally for weeks or more after electroporation.

### Viral Tropism

In a preferred embodiment, virus derived from the transgene encoding vector(s), therapeutic vector(s) and/or other transgenic vector(s) of this invention is pseudotyped with vesicular stomatitis virus envelope glycoprotein to enable concentration of the virus to high titers and to facilitate infection of CD34+ cells.

### Sequence Selection

The use of any sequence with 70% or greater identity (or complementarity) to any sequence referred to a transgene sequence named herein (searchable using the Entrez-Pubmed database) is covered by the invention if utilized in the manner described in the present invention.

The current invention also relates in part to a genetic vector that includes sequences (and/or their corresponding proteins) capable of markedly reducing the susceptibility of mammalian cells to infection by HIV 1 and HIV-2 viruses (both together referred to herein as HIV).

The current invention discloses the novel combination of synthetic oligonucleotides to reduce the expression of genes critical to the HIV/AIDS disease process.

The desirability of combining synthetic oligonucleotides to effect co-receptor “knock down” with expression of TAR and RRE decoy sequences (and/or their corresponding proteins) arises from the proposition, expressed herein, that combining multiple gene therapy approaches simultaneously targeting 1) HIV infection, 2) HIV transcription, and 3) HIV replication in individual cells is likely to produce superior therapeutic benefits than any of these approaches in isolation.

Therapeutic vector(s) express “beneficial sequence(s)” intended to render transfected or infected cells less capable of sustaining HIV replication and transcription. The genetic vector expressing “beneficial sequence(s)” as well as any virus derived from such genetic vector, are herein termed “therapeutic vector”.

In a preferred embodiment, the therapeutic vector(s) contains one or more synthetic oligonucleotide sequences driven by one or more promoters so as to reduce expression of one or more of HIV vif, vpr, vpu, vpx, tat and/or nef, and/or homologous sequences in FIV or SW.

In a preferred embodiment, the therapeutic vector(s) contains one or more synthetic oligonucleotide sequences driven by one or more promoters so as to reduce expression of one or more of HIV rev, gag, pol, env, protease and/or homologous sequences in FIV or SIV.

Infection by HIV is believed to involve receptors termed “HIV receptors.” There may be multiple such receptors, some of which may be termed “HIV co-receptors.” As discussed herein, HIV co-receptors include CXCR4 and CCR5.

The present invention is directed in part to the genetic engineering, aka genetic modification, of cells susceptible to infection by HIV or capable of propagating HIV. Such cells are herein termed “target cells”.

In one embodiment, a cell comprising a mutation or deletion in the CCR5 and/or CCRX4 co-receptors, and/or other co-receptors is developmentally-activated according to the methods described herein to provide pluripotent cells, pluripotent like cells, multipotent cells, hematopoietic progenitors and stem cells, T cells and/or macrophages such that the resulting T cells and macrophages were HIV-resistant.

In a further embodiment, the CCR5 mutation or deletion is a 32 base pair deletion or other rendering the CCR5 gene non-functional.

In a further embodiment, CRISPR/CAS9 or other site-directed mutational methods known to the art to produce mutation or deletion in the CCR5 and/or CCRX4 co-receptors.

The present invention also provides a composition and method for using therapeutic viral vectors to reduce the susceptibility of mature or immature target cells, leukocytes, blood cells, any stem/progenitor cells, and/or their progeny (including DAdC) to infection by HIV.

It follows that the present invention also provides a composition and method for using therapeutic viral vectors to reduce the susceptibility of developmentally activated cells, induced cells, reprogrammed cells, induced multipotent cells, induced pluripotent cells, and/or their progeny to infection by HIV.

It is a further objective of this invention to reduce the ability of mature or immature target cells, stem/progenitor cells, (including developmentally activated cells, induced cells, reprogrammed cells, induced multipotent cells, induced pluripotent cells) and/or their progeny to sustain immunodeficiency virus replication and transcription.

It is another objective of this invention to achieve efficient, long-term expression of the therapeutic sequences (and/or their corresponding proteins) in mature or immature target cells, other quiescent cells, stem/progenitor cells, and/or their progeny.

In one aspect, this invention provides a method for preventing or treating HIV infection. The method involves transplanting stem cells transfected with therapeutic vector(s) or sequence(s), into patients or subjects with HIV infection.

Beneficial sequence(s) may be ones that reduce the ability of HIV to infect a cell, transcribe viral DNA, or replicate within an infected cell, or which enhances the ability of a cell to neutralize HIV infection.

In certain embodiments, the beneficial sequence(s) represent synthetic oligonucleotide(s) which interfere with HIV entry, including one or more selected from siRNA, shRNA, antisense RNA or miRNA directed against any of the HIV co-receptors (including, but not limited to, CXCR4, CCR5, CCR2b, CCR3, and CCR1).

In a preferred embodiment, the therapeutic vector(s) includes synthetic oligonucleotides targeting one or more HIV co-receptors including CXCR4, CCR5, CCR1, CCR2, CCR3, CXCR6 and/or BOB. In another preferred embodiment the therapeutic vector(s) includes synthetic oligonucleotides targeting the major HIV co-receptors CXCR4 and/or CCR5

In a further preferred embodiment, the therapeutic vector(s) includes synthetic oligonucleotides targeting one or more HIV enzymes such as HIV reverse transcriptase, integrase and protease.

Appropriate sequences (and/or their corresponding proteins) for the synthetic oligonucleotides are those 1) predictable by computer algorithms to be effective in reducing targeted sequences (or their corresponding proteins), and 2) capable of successfully reduce the amount of targeted enzyme by >70% in standard quantitative RNA assays and in assays of enzymatic activity or to a lesser but therapeutic degree.

The phrase “targeted sequence” indicates that a particular sequence has a nucleotide base sequence that has at least 70% identity to a viral genomic nucleotide sequence or its complement (e.g., is the same as or complementary to such viral genomic sequence), or is a corresponding RNA sequence. In particular embodiments of the present invention, the term indicates that the sequence is at least 70% identical to a viral genomic sequence of the particular virus against which the oligonucleotide is directed, or to its complementary sequence. Any of the various types of synthetic oligonucleotides may be expressed via therapeutic vector transfection, and the current invention is directed to all possible combinations of such oligonucleotides.

In a preferred embodiment, the synthetic oligonucleotide sequences are driven by target cell, specific promoter(s). In another preferred embodiment, the synthetic oligonucleotide sequences are driven by U6 promoter(s). Synthetic oligonucleotides, by the same token, may be included in the same therapeutic vector(s) with decoy RNA.

### Decoy RNA

Decoy RNA are sequences (and/or their corresponding proteins) of RNA that are effective at binding to certain proteins and inhibiting their function. In a preferred embodiment, the therapeutic vector(s) comprise(s) multiple decoy RNA sequences (or their corresponding proteins). In a further embodiment the decoy RNA sequences (and/or their corresponding proteins) are flanked by sequences (and/or their corresponding proteins) that provide for stability of the decoy sequence.

In another preferred embodiment the decoy RNA sequences (and/or their corresponding proteins) are RRE and/or TAR decoy sequences (or their corresponding proteins). In a preferred embodiment, the RRE and TAR decoy sequences (and/or their corresponding proteins) are HIV-2 derived TAR and RRE sequences (or their corresponding proteins). In another preferred embodiment the decoy sequences (and/or their corresponding proteins) also include Psi element decoy sequences (or their corresponding proteins). In a preferred embodiment, the decoy sequences (and/or their corresponding proteins) are each driven by a U6 promoter.

In another preferred embodiment, the decoy sequences (and/or their corresponding proteins) are driven by target-cell specific promoters. In a preferred embodiment, the therapeutic vector targets multiple stages of the HIV life cycle by encoding synthetic nucleotide sequence(s) in combination with HIV-2 TAR and/or RRE decoy sequences (or their corresponding proteins).

In another preferred embodiment, the vector includes miRNA oligonucleotide sequences (or their corresponding proteins). In another preferred embodiment, the vector includes shRNA oligonucleotide sequences (or their corresponding proteins). In another preferred embodiment, the vector includes siRNA oligonucleotide sequences (or their corresponding proteins). In another preferred embodiment, the vector includes RNAi oligonucleotide sequences (or their corresponding proteins). In another preferred embodiment, the vector includes ribozyme sequences (or their corresponding proteins). In another preferred embodiment, the vector includes a combination of synthetic oligonucleotide classes. In a further embodiment, the synthetic nucleotide sequences (and/or their corresponding proteins) target HIV co-receptors such as CCR5, CXCR4, etc. In a further embodiment, the synthetic nucleotide sequences (and/or their corresponding proteins) target HIV enzymes such as integrase, protease, reverse transcriptase, TAT, etc.

In a further embodiment, the ribozyme sequences (and/or their corresponding proteins) target HIV co-receptors such as CCR5, CXCR4, etc, or HIV enzymes such as integrase, protease, reverse transcriptase, TAT, etc. In a preferred embodiment, virus is generated using the therapeutic vector(s) and the virus is pseudotyped. In a preferred embodiment, virus is generated using the therapeutic vector(s) and the virus is not pseudotyped and the virus shows native HIV tropism. In a preferred embodiment, the therapeutic vector(s) is a viral vector. In a preferred embodiment, the therapeutic vector(s) is a lentiviral vector. In a preferred embodiment, the therapeutic vector(s) is a third-generation lentiviral vector.

In a preferred embodiment, the therapeutic vector(s) includes a combination of synthetic oligonucleotide classes. In a preferred embodiment, synthetic nucleotide sequence expression is driven by the EF-1 alpha promoter or other target-cell appropriate promoters. In a preferred embodiment, synthetic nucleotide sequence expression is driven by the U6 promoter or other target-cell appropriate promoters. In a preferred embodiment, synthetic nucleotide sequence expression is driven by a combination of EF-1 alpha and U6, and/or other target-cell appropriate promoters. In a preferred embodiment, EF-1 alpha drives miRNA expression while the U6 promoter drives RNA decoy expression. In a preferred embodiment, EF-1 alpha drives siRNA sequence expression while the U6 promoter drives RNA decoy expression. In a preferred embodiment, EF-1 alpha drives shRNA sequence expression while the U6 promoter drives RNA decoy expression.

In a preferred embodiment, the therapeutic vector(s) include synthetic oligonucleotides (e.g. multiple miRNA sequences)) directed against CXCR4, multiple sequences directed against CCR5, an HIV-2 RRE decoy sequence and an HIV-2 TAR decoy sequence, and the vector is a viral vector. See FIG. 15.

In a preferred embodiment, treatment involving the therapeutic vector(s) is combined with other modes of antiretroviral therapy including pharmacological therapies. Antiretroviral therapies appropriate for combination with the therapeutic vector(s) are those that have additive or synergistic effects in combination with the therapeutic vector.

Cells targeted for gene therapy in HIV may include, but are not necessarily be limited to developmentally-activated cells, mature peripheral blood T lymphocytes, monocytes, tissue macrophages, T cell progenitors, macrophage-monocyte progenitor cells, and/or multipotent hematopoietic stem cells, such as those found in umbilical cord blood, peripheral blood, and occupying bone marrow spaces.

The present invention also relates to transfection of CD4+ T cells, macrophages, T cell progenitors, macrophage-monocyte progenitors, CD 34+ stem/progenitor cells and/or any other quiescent cell, dividing cell, stem cell or progenitor cell capable of differentiation in vitro or in vivo into HIV target cells, CD4+ T cells, macrophages, T cell progenitors, macrophage-monocyte progenitors, and/or CD 34+ stem/progenitor cells. Transfected cells, therefore, can be endogenous cells in situ, or exogenous cells derived from other body regions or even other individual donors. Cells selected for this purpose are herein termed “selected cells”.

By the same token, developmentally-activated, e.g. self-renewing, multipotent and/or pluripotent stem cells (including reprogrammed and induced pluripotent cells) represent another logical target for HIV gene therapy, and their use is specifically covered by the present invention.

In one embodiment of this process, selected cells (e.g. hematopoietic stem cells, skin stem cells, umbilical cord cells, primordial germ cells (PGCs), spermatogonia, any accessible somatic cell, etc.) are 1) propagated in culture using one or more cytokines such as steel factor, leukemia inhibitory factor (LIF), cardiotropin-1, IL-11, IL-6, IL-6 R, GP-130, CNTF, IGF-I, bFGF, and/or oncostatin-M and 2) transfected with the therapeutic vector(s) or beneficial sequence(s) prior to differentiation using any methods known to the art, such as those described in U.S. Pat. No. 5,677,139 herein incorporated by reference, or by methods analogous to U.S. Pat. No. 5,677,139 with respect to other target cells. In separate embodiments, it may be desirable to perform the various steps prior to developmental activation.

The population of target cells may include somatic cells, stem cells and progenitor cells. Stem cells may be derived from existing cell lines or isolated from stored, banked, or cryopreserved sources. Typical sources of stem cells include marrow, peripheral blood, placental blood, amniotic fluid, umbilical cord blood, adipose tissue, non-human embryos, etc.

Somatic cells, especially circulating leukocytes and other non-progenitor/stem cells may likewise be subjected to the same culture conditions as described above for stem/progenitor cells effective that they acquire stem/progenitor cell properties as a result.

The invention also discloses the production (e.g. US Patent Application 20030099621) of target cells from stem/progenitor cells that may be made relatively resistant to HIV infection and/or HIV replication.

It is understood, however, that any method of differentiating previously propagated stem/progenitor/leukocyte cells into the desired target cells may be employed within the scope of the invention so long as functional target cells relatively resistant to HIV infection and/or HIV replication/and/or HIV transcription are produced.

In a preferred embodiment, the therapeutic viral vector is packaged with one or more envelope proteins from native HIV viruses conferring upon the therapeutic virus the capacity to infect any cell that native HIV strains are capable of infecting.

Cells selected for use in this invention will, in some instances, be accessible (e.g. umbilical cord stem cells, bone marrow stem cells, spermatogonia and primordial germ cells of the testis, stem cells isolated from amniotic fluid, stem cells isolated from the skin, etc.). Such cells can be isolated from the tissues in which they reside by any means known to the art.

Other selected cells may comprise reprogrammed cells, induced multipotent cells, induced pluripotent cells, etc.

In accordance with an aspect of the present invention, there is provided a method of producing a desired cell line, cell type, or cell class from the selected cells. Generally, the method comprises culturing the selected cells and/or their progeny under conditions which promote growth of the selected cells at an optimal growth rate. The resulting cell population is then cultured under conditions which promote cell growth at a rate which is typically less than the optimal rate, and in the presence of an agent promoting differentiation of the cells into the desired cell line, cell type, or cell class (e.g. CD4+ T cells).

The present invention also discloses the propagation of the selected cells and/or their progeny in culture, before or after transfection with (or overexpression of) the therapeutic vector, by any means known to the art (e.g. US Patent Application 20060099177). Such methods also include incubation with LIF, steel factor, Il-6, IL-7, oncostatin-M and/or cardiotropin-1 and other growth enhancing cytokines, etc.

The present invention further discloses the directed differentiation of cells transfected with the therapeutic vector(s) into desired cell types by further incubation in media containing the appropriate cytokines and growth factors such as colony stimulating factors such as M-CSF (CSF-1), GM-CSF, IL-7, any cytokine promoting CD4+ T cell differentiation, etc.

The present invention teaches transfection and/or CRSPR/Cas9 for genetic modification, including genetic correction. Genetic modification of selected cells and target cells, whether they be exogenous cells or endogenous cells can be performed according to any published or unpublished method known to the art (e.g. U.S. Pat. Nos. 6,432,711, 5,593,875, 5,783,566, 5,928,944, 5,910,488, 5,824,547, CRISPR-Cas: A Laboratory Manual (2016, Genome Editing Using Engineered Nucleases and Their Use in Genomic Screening (2017), etc.) or by other generally accepted means. Suitable methods for transforming host cells can be found in Sambrook et al. (Molecular Cloning: A Laboratory Manual, 2nd Edition, Cold Spring Harbor Laboratory press (1989)), and other laboratory textbooks.

Successfully transfected cells can be identified by selection protocols involving markers such as antibiotic resistance genes in addition to RNA expression assays and morphological analyses. Clones from successfully transfected cells, expressing the appropriate exogenous DNA at appropriate levels, can be preserved as cell lines by cryopreservation (utilizing any appropriate method of cryopreservation known to the art).

Selectable markers (e.g., antibiotics resistance genes) may include those which confer resistance to drugs, such as G418, hygromycin, ampicillin and blasticidin, etc. Cells containing the gene of interest can be identified by drug selection where cells that have incorporated the selectable marker gene survive.

A theoretical basis for the embodiments of the invention is described herein, however, this discussion is not in any way to be considered as binding or limiting on the present invention. Those of skill in the art will understand that the various embodiments of the invention may be practiced regardless of the model used to describe the theoretical underpinnings of the invention.

The invention will now be described and illustrated with respect to the following examples; however, the scope of the present invention is not intended to be limited thereby.

### Example 1: Construction of the Transgenic Vectors Suitable for Use in the Present Invention

Suitable EGFP-Numb and EGFP-Numblike, and EGFP-X lentiviral vectors (where X is any transgene described in the present invention) can be produced by cloning into an appropriate viral vector (e.g. the two-gene HIV-EGFP-HSA vector (Reiser et al., 2000)). Adapter primers can be selected for PCR amplification of Numblike and Numb isoform cDNAs and cloning into a genetic vector. In preparation for cloning, the gene vector is digested with enzymes. Subsequently, the cDNA for each transgene is inserted into the nef coding region previously occupied by the HSA cDNA-EGFP (enhanced green fluorescent protein) and a cell population-appropriate promoter (e.g. CMV ie or EF1alpha) having been previously inserted into the viral, gag-pol coding region. The lack of a functional integrase gene in these vectors renders them integrase-deficient and episomal, and is the consequence of inserting the EGFP expression cassette consisting of EGFP sequences and the CMV IE promoter into the region normally occupied the gag-pol genes (FIG. 6A of Reiser et al., 2000; see FIG. 3D herein). Such integrase-deficient vectors can be readily introduced using a variety of standard transfection techniques (e.g. electroporation, chemically mediated transfection, fusogenic or non-fusogenic liposomes, lipofectamine, nanocapsules, nanovaults, etc.)—methods which allow high capacity integrase-deficient lentiviral vectors to be utilized without genomic integration and random alteration of the genome.

When cell transduction is mediated by virus (i.e. viral particles) pol deletion interferes with reverse transcription of viral RNA to DNA. Accordingly, pol deletion results in a non-integrating or integration deficient lentivector. This is because “Integrase . . . is involved in the reverse transcription of HIV-1 RNA and nuclear import of the pre-integration complex (PIC) (Gallay et al., 1997; Zhu et al., 2004; Philpott and Thraser, 2007).

However, when one skilled in the art chooses not to use infection and instead chooses electroporation to introduce DNA lentiviral vectors to the cells, as taught by the applicant, no reverse transcription is necessary; and because the pol gene is deleted, no integrase enzyme is present in the cell to support integration. It has been taught by Wanisch and Yanez-Munoz (2009) that, “HIV-1 circles are considerably stable after infection, with progressive vector episome dilution due to cell division. Thus, the apparent decrease in circularized HIV-1 DNA after infection of CD4+ MT-2 or SupT1 T-cells is the result of ongoing cell division causing the dilution of nonreplicating viral episomes in the cell population. Episomes are stable in macrophages for at least 21 days (ref. 36), while a turnover of episomes has been observed in vivo in human peripheral blood mononuclear cells over the course of several weeks, suggesting that they can be slowly degraded.” (Wanisch and Yanez-Munoz, 2009). Accordingly, those skilled in the art recognize that integration deficient lentivectors may persist episomally for weeks or more after electroporation.

Genetic constructs may include a vector backbone, and a transactivator which regulates a promoter operably linked to heterologous nucleic acid sequences (or their corresponding proteins). Examples of retroviral vectors which may be employed include, but are not limited to, those derived from Moloney Murine Leukemia Virus, Moloney Murine Sarcoma Virus, and Rous Sarcoma Virus, FIV, and HIV. Appropriate expression vectors are those that may be employed for transfecting DNA or RNA into eukaryotic cells. Such vectors include, but are not limited to, prokaryotic vectors such as, for example, bacterial vectors; eukaryotic vectors, such as, for example, yeast vectors and fungal vectors; and viral vectors, such as, but not limited to, lentiviral vectors, adenoviral (Lin et al., 2007) vectors, adeno-associated viral vectors, and retroviral vectors.

The replication incompetent pcDNA 6.2/EmGFP-Bsd/V5-DEST vector is an example of an appropriate expression vector (Invitrogen) and allows expression of synthetic oligonucleotides (e.g. miRNAs) transferred from the pcDNA 6.2 GW/miR vector that have the capacity to cleave targeted sequences (or their corresponding proteins). These vectors include flanking and loop sequences (and/or their corresponding proteins) from endogenous miRNA to direct the excision of the engineered miRNA from a longer Pol II transcript (pre-miRNA).

Combining multiple miRNA sequences (and/or their corresponding proteins) directed against specific endogenous RNA species increases the likelihood of success in reducing target sequence expression. miRNA sequences (and/or their corresponding proteins) may be operably linked to regulable or tissue specific promoters.

By utilizing lentiviral vectors for gene expression, the resulting transgene encoding vector(s) and/or other transgenic vector(s) of this invention, becomes capable of stably transducing both dividing and non-dividing cell types.

Moreover, 2nd and 3d generation, integrase-deficient lentiviral vectors provide a non-integrating, episomal vector suitable, along with adenoviral (Lin et al., 2007), AAV, hybrid vectors, plasmid DNA, etc. for use in the present invention. (See FIG. 1D).

In a preferred embodiment, the resulting Numb/Numblike encoding vector(s), and/or other transgenic vector(s) of this invention contain multiple synthetic oligonucleotide sequences driven by one or more promoters so as to reduce expression of specific numb isoforms and/or numblike (FIG. 3D).

### Example 2: Another Example of a Suitable Vector is a Retroviral Vector

Retroviruses are RNA viruses that contain an RNA genome. The gag, pol, and env genes are flanked by long terminal repeat (LTR) sequences (or their corresponding proteins). The 5′ and 3′ LTR sequences (and/or their corresponding proteins) promote transcription and polyadenylation of mRNAs.

The retroviral vector may provide a regulable transactivating element, an internal ribosome reentry site (IRES), a selection marker, and a target heterologous gene operated by a regulable promoter.

Alternatively, multiple sequences (and/or their corresponding proteins) may be expressed under the control of multiple promoters. Finally, the retroviral vector may contain cis-acting sequences (and/or their corresponding proteins) necessary for reverse transcription and integration. Upon infection, the RNA is reverse transcribed to DNA that integrates efficiently into the host genome. The recombinant retrovirus of this invention is genetically modified in such a way that some of the retroviral, infectious genes of the native virus have been removed and in certain instances replaced instead with a target nucleic acid sequence for genetic modification of the cell. The sequences (and/or their corresponding proteins) may be exogenous DNA or RNA, in its natural or altered form.

### Example 3: Example Methods for Generation of Numb/Numblike Encoding Vector(s), and/or Other Transgenic Vector(s) of this Invention

The methods for generation of the resulting Numb/Numblike encoding vector(s), and/or other transgenic vector(s) of this invention include those taught in Invitrogen's Viral Power Lentiviral Expression Systems Manual, 2007. Briefly, the EmGFP-bsd cassette is cloned as a Pm1I-B1pI fragment into the pLenti6/R4R2/V5-DEST vector, while the miR-long (PRR+) numb isoform or miR-short numb isoform/numblike cassettes are simultaneously transferred by BP reaction into pDONR221. Then the regulable promoter(s) and miR-isoform cassettes are Multi-site LR crossed into the modified pLenti6/EmGFP-bsd/R4R2-DESTvector.

Multiple vectors can be generated in this manner comprising different combinations of synthetic oligonucleotides and transgene cassettes.

The pLenti6/R4R2/V5-DEST vector sequence corresponds to (SEQ ID NO: 1).

### Example 4: Additional Methods for Generation of Therapeutic Vector(s)

“Packaging cell lines” derived from human and/or animal fibroblast cell lines result from transfecting or infecting normal cell lines with viral gag, pol, and env structural genes. On the other hand, packaging cell lines produce RNA devoid of the psi sequence, so that the viral particles produced from packaging cell do not contain the gag, pol, or env genes. Once the therapeutic vector's DNA containing the psi sequence (along with the therapeutic gene) is introduced into the packaging cell, by means of transfection or infection, the packaging cell may produce virions capable of transmitting the therapeutic RNA to the final target cell (e.g. a CD4+ cell).

The “infective range” of the therapeutic vector(s) is determined by the packaging cell line. A number of packaging cell lines are available for production of virus suitable for infecting a broad range of human cell types. These packaging cell lines are nevertheless generally capable of encapsidating viral vectors derived from viruses that in nature usually infect different animal species. For example, vectors derived from SIV or MMLV can be packaged by GP120 encapsidating cell lines.

### Example 5: Growth Medium for Selected Cells

Selected cells can be expanded/grown in Dulbecco's modified Minimal Essential Medium (DMEM) supplemented with glutamine, beta.-mercaptoethanol, 10% (by volume) horse serum (unless eliminated in a serum free medium), and human recombinant Leukemia Inhibitory Factor (LIF). LIF replaces the need for maintaining selected cells on feeder layers of cells, (which may also be employed) and is essential for maintaining selected cells in an undifferentiated, multipotent, or pluripotent state, such cells can be maintained in Dulbecco's modified Minimal Essential Medium (DMEM) supplemented with glutamine, beta.-mercaptoethanol, 10% (by volume) horse serum, and human recombinant Leukemia Inhibitory Factor (LIF). The LIF replaces the need for maintaining cells on feeder layers of cells, (which may also be employed) and is essential for maintaining cells in an undifferentiated state (per U.S. Pat. No. 6,432,711).

In order to initiate the differentiation of the selected cells into neuronal cells, the cells are trypsinized and washed free of LIF, and placed in DMEM supplemented with 10% fetal bovine serum (unless eliminated in a serum free medium). After resuspension in DMEM and 10% FBS, 1×10⁶ cells are plated in 5 ml DMEM, 10% FBS, 0.5 microM retinoic acid in a 60 mm Fisher bacteriological grade Petri dishes, where the cells are expected to form small aggregates. Aggregation aids in proper cell differentiation. High efficiency transfection with (or overexpression of) appropriate neuronal transcription factors and small RNAs can occur before or after plating in DMEM, FBS, and retinoic acid. (See U.S. Pat. Nos. 6,432,711 and 5,453,357 for additional details).

### Example 6: HLA Matching

Selected Cells (e.g. Umbilical Cord Blood or Cells from any Other Suitable Source and/or their Progeny), can be Screened, Genetically-Modified (Optional), Expanded, and Induced to Begin Differentiating into the Desired Cell Type(s) (Optional). The Cells are then Transplanted According to Standard Stem Cell Transplantation Protocols. In Certain Instances, Cells May be Transplanted into Patients or Subjects without HLA Matching

### Example 7

In Some Rare Instances, it May be Appropriate to Introduce Transgene Encoding Vectors into Patients or Subjects in Order to Stimulate or Inhibit Cellular Division or Cellular Differentiation, In Vivo (e.g. in Cancer). Short Numb and Numblike Block Cancerous Behavior and May be Introduced to Cancerous Cells Using the Vectors Taught Herein

### Example 8: Genetic Modification of Selected Cells

In vitro genetic modification of exogenous cells or patient's endogenous cells can be performed according to any published or unpublished method known to the art (e.g. U.S. Pat. Nos. 6,432,711, 5,593,875, 5,783,566, 5,928,944, 5,910,488, 5,824,547, etc.) or by other generally accepted means. Suitable methods for transforming host cells can be found in Sambrook et al. (Molecular Cloning: A Laboratory Manual, 2nd Edition, Cold Spring Harbor Laboratory press (1989)), and other laboratory textbooks.

Successfully transfected cells are identified by selection protocols involving markers such as antibiotic resistance genes in addition to RNA expression assays and morphological analyses. Clones from successfully transfected cells, expressing the appropriate exogenous DNA at appropriate levels, can be preserved as cell lines by cryopreservation (utilizing any appropriate method of cryopreservation known to the art).

Selectable markers (e.g., antibiotic resistance genes) may include those conferring resistance to drugs, such as G418, hygromycin and methotrexate. Cells containing the gene of interest can be identified by drug selection where cells that have incorporated the selectable marker gene survive, and others die.

The current invention discloses the selection of genetically-modified cells as “selected cells” of the invention. The term genetic modification refers to alteration of the cellular genotype by introducing natural or synthetic nucleic acids into selected cells and/or their progeny or immortalized cell lines and/or their progeny by any means known to the art. Alternatively culture conditions that induce permanent changes in gene expression patterns are considered herein to represent genetic modification. Modification of stem cells, whether they be derived from the host brain, endogenous donor sources, exogenous donor sources, or cell lines, represents a feasible approach to the treatment of certain human diseases, especially those of the human nervous system.

Genetic modifications covered by this disclosure include, but are not limited to: genetic modifications performed in vivo; modifications that alter the activity or amount of metabolic enzymes expressed by endogenous or exogenous selected cells and/or their progeny; modifications which modulate angiogenesis and/or wound healing; modifications which alter the activity, amount, or antigenicity of cellular proteins; modifications which alter the activity or amount of proteins involved in signal transduction pathways; modifications which alter HLA type; modifications which alter cellular differentiation; modifications which alter neoplastic potential; modifications which alter cellular differentiation; modifications which alter the amount or activity of structural proteins; modifications which alter the amount or activity of membrane associated proteins (structural or enzymatic); modifications which alter the activity or amount of proteins involved in DNA repair and chromosome maintenance; modifications which alter the activity or amount of proteins involved in cellular transport; modifications which alter the activity or amount of enzymes; modifications which alter the activity or amount of proteins involved in synapse formation and maintenance; modifications which alter the activity or amount of proteins involved in neurite outgrowth or axon outgrowth and formation; modifications altering the amount or activity of antioxidant producing enzymes within the cell; modifications which lead to altered post-translational modification of cellular proteins; modifications which alter the activity or amount of proteins involved in other aspects of cellular repair, and alterations which increase the lifespan of the cell (such as production of telomerase). Such proteins as those mentioned above may be encoded for by DNA or RNA derived from the human genome or other animal, plant, viral, or bacterial genomes. This invention also covers sequences (and/or their corresponding proteins) designed de novo.

In some embodiments, the cells of the present invention, once activated and/or modified according to the methods described herein, are utilized as delivery vehicles for biologic drugs, especially proteins.

In some embodiments, the cells of the present invention are selected or modified, aka engineered, aka modified, to for the expression of useful biological molecules.

In some embodiments, the cells of the present invention are engineered, aka modified, to express, overexpress, and/or secrete proteins, nucleic acids, and/or other molecules with biological activity.

In some embodiments, the molecules are one associated with wound healing and angiogenesis (Tam et al., 2015; Peake et al., 2014), or ones in taught in US20100324127A1, US20040115769A1, and US20100324127A1: progranulin, granulin, angiogenin, angiopoietin-1, del-1 protein, fibroblast growth factors such as acidic FGF (also known as aFGF or FGF-1) and basic FGF (also known as bFGF or FGF-2), follistatin, granulocyte colony-stimulating factor (G-CSF), hepatocyte growth factor (HGF), interleukin-8 (IL-8), leptin, midkine, placental growth factor, platelet-derived endothelial growth factor (PD-ECGF), platelet-derived growth factor (PDGF), pleiotrophin (PTN), progranulin, proliferin, transforming growth factor alpha (TGF-α), transforming growth factor beta (TGF-β), tumor necrosis factor alpha (TNF-α), vascular endothelial growth factor (VEGF), and vascular permeability factor (VPF).

In some embodiments, the useful biological molecule or molecules with biological activity are selected from

In some embodiments, the useful biological molecules or molecules with biological activity are antibodies or T cell receptors useful in cancer or an infection (e.g. for immunotherapy). In some embodiments, the cells of the present invention are genetically modified (aka engineered) to express one or more antibodies and/or T cell receptors (or portions thereof).

In some embodiments, the cells of the present invention are genetically modified (aka engineered) to express two or more antibodies and/or T cell receptors (or portions thereof).

In some embodiments, the cells of the present invention are genetically modified (aka engineered) to express multiple antibodies and/or multiple T cell receptors (or portions thereof).

In some embodiments, the useful biological molecule or molecule with biological activity in one or more neutralizing antibody, other antibody, or T cell receptor and the invention thereby represents a “direct” or “immediate” vaccine and performs a function similar to a vaccine.

In some embodiments, the neutralizing antibody or antibodies neutralize a coronavirus (e.g. SARS-CoV-2, aka Covid-19) such as the neutralizing antibodies described by Wang et al., (2020); Cao et al., (2020); Rogers s t al., (2020), and Brouwer et al, (2020).

In some embodiments, the useful biological molecule or molecule with biological activity in one or more antibody or T cell receptor and the invention thereby represents an immunotherapy

In some embodiments, the useful biological molecules or molecules comprise or consist of one or more small RNA.

In some embodiments, the useful biological molecules or molecules with biological activity consist of one or more selected from the Hormonal, Enzymatic Protein, Structural Protein, Defensive Protein, Storage Protein, Transport Protein, Receptor Protein and Contractile Protein classes.

In some embodiments, the useful biological molecules or molecules with biological activity consist of or comprise nucleic acids or proteins corresponding to ones deficient in a patient.

In some embodiments, the useful biological molecules or molecules with biological activity do not consist of or comprise nucleic acids or proteins.

In some embodiments, the useful biological molecules or molecules with biological activity consist of or comprise nucleic acids or proteins corresponding to one or more selected from collections of genes associated with human diseases (see DisGeNET version 7 which lists 21,671 such genes at www.disgenet.org; see also Piñero et al., 2019; Piñero et al., 2016; and Piñero et al., 2015).

In some embodiments, the cells of the present invention are engineered, aka modified, to express, overexpress, and/or secrete proteins, nucleic acids, and/or other molecules with biological activity.

In addition, this invention relates to the in situ, genetic modification of selected cells and/or their progeny cells for the treatment of disease. Endogenous stem cells may be modified in situ by direct injection or application of DNA or RNA vectors, including viruses, retroviruses, liposomes, etc, into the substance of the tissue or into the appropriate portion of the ventricular system of the brain. Since 1992, the applicants have modified thousands of stem/progenitor cells and many thousand progeny cells in this manner. Applicants' data shows that this manner of modifying progenitor cells results in a tremendous variety of modified cell types throughout the nervous system, and has never resulted in adverse effects.

### Example 9: Introduction of Genetic Vectors into the Host

In a preferred embodiment, endogenous cells are transfected with vectors such as those described herein in vivo by introduction of the therapeutic vector(s) into the host blood, tissues, nervous system, bone marrow, etc. The greatest benefit may be achieved by modifying a large number of endogenous target cells. This may be accomplished by using an appropriately-sized, catheter-like device, or needle to inject the therapeutic vector(s) into the venous or arterial circulation, into a specific tissue, such as muscle tissue, or into the nervous system. In a preferred embodiment, the virus is pseudotyped with VSV-G envelope glycoprotein and native HIV-1 env proteins.

### Example 10: Injection into the Nervous System

Transplantation of selected cells (from either the growth or differentiation media) into the fetal nervous system or genetic modification of endogenous fetal cells utilizing genetic vectors may be accomplished in the following manner: Under sterile conditions, the uterus and fetuses are visualized by ultrasound or other radiological guidance. Alternatively the uterus may be exposed surgically in order to facilitate direct identification of fetal skull landmarks. Selected cells can then be introduced by injection (using an appropriately-sized catheter or needle) into the ventricular system, germinal zone(s), or into the substance of the nervous system. Injections may be performed in certain instances, through the mother's abdominal wall, the uterine wall and fetal membranes into the fetus. The accuracy of the injection is monitored by direct observation, ultrasound, contrast, or radiological isotope based methods, or by any other means of radiological guidance known to the art.

Under appropriate sterile conditions, direct identification of fetal skull landmarks is accomplished visually as well as by physical inspection and palpation coupled with stereotaxic and radiologic guidance. Following cell culture, appropriate amounts of the selected or differentiating cells can then be introduced by injection or other means into the ventricular system, germinal zones, or into the substance of the nervous system. The accuracy of the injection may be monitored by direct observation, ultrasound, or other radiological guidance.

In certain, neurological diseases of the adult nervous system, such as Huntington's disease and Parkinson's disease, cells of a specific portion of the brain are selectively affected. In the case of Parkinson's disease, it is the dopaminergic cells of the substantia nigra. In such regionally-specific diseases affecting adults, localized transplantation of cells may be accomplished by radiologically-guided transplantation of differentiating cells under sterile conditions. Radiologic guidance may include the use of CT and/or MRI, and may take advantage of contrast or isotope based techniques to monitor injected materials.

In certain neurologic diseases, such as some metabolic storage disorders, cells are affected across diverse regions of the nervous system, and the greatest benefit may be achieved by genetically-modifying endogenous cells or introducing selected cells of the present invention (either from the growth culture media or the differentiating medium) into the tissue in large numbers in a diffuse manner. In the nervous system, these diseases may be best approached by intraventricular injections (using an appropriately-sized, catheter-like device, or needle) (especially at early stages of development) which allows diffuse endogenous cell modification or diffuse engraftment of selected cells isolated from the growth and/or differentiation media. Nevertheless, injection of the cells into the circulatory system for the same purpose is also covered. However, with regard to any disorder affecting multiple organs or the body diffusely (e.g. lysosomal storage disorders, hemoglobinopathies, muscular dystrophy), the cells isolated from the growth and/or differentiation media may also be preferentially introduced directly into the circulation and/or visceral organs, such as the liver, kidney, gut, spleen, adrenal glands, pancreas, lungs, and thymus using endoscopic guidance and any appropriately-sized, catheter-like device, allowing diffuse engraftment of the cells throughout the body, as well as specific introduction and infiltration of the cells into the selected organs.

### Example 11: Delivery of Cells by Injection into the Circulatory Stream and Organs

Diseases of one organ system may be treatable with genetically modified cells from a separate organ system. Also, in some instances, it may become apparent that the selected cells may integrate and differentiate on their own, in vivo, in sufficient numbers if they are injected into blood stream either arterial, venous or hepatic, after culturing in the growth and/or differentiation media. This approach is covered by the present invention. The treatment of diffuse muscle (e.g. muscular dystrophies), organ, tissue, or blood disorders (e.g. Hereditary Spherocytosis, Sickle cell anemia, other hemoglobinopathies, etc,) may, for instance, involve the injection of cells isolated from the growth media or differentiating media into the patient, especially the patient's circulation. This approach is also believed to ameliorate ischemic injuries such as myocardial infarction, stroke, etc., as well as traumatic injuries to brain and other tissues. Injection of such cells produced by the current invention, directly into the circulation, by needle or catheter, so that the cells are enabled to “home” to the bone marrow, muscle, kidneys, lungs, and/or any other organ system, as well as injection directly into the bone marrow space is suitable for the practice of the present invention. Likewise, injection of the cells directly into a lesion site with or without radiologic, ultrasonic or fluoroscopic guidance is also suitable for the practice of the present invention.

Methods of isolating selected cells useful in the present invention include those described by Zhao et al., 2006.

In a preferred embodiment, genetic vectors encoding numblike and/or numb isoforms comprise regulable promoters operably linked to the Numb or numblike transgenes (or their corresponding proteins).

In another preferred embodiment, the mode of transfection may be selected from those modes of transfection that provide for transient rather than permanent expression of the numblike and numb isoforms.

### Example 12. Example Genetic Modifications

Hundreds of diseases and clinical conditions are able to be treated and/or ameliorated by the methods of the present invention wherein a gene deficient in a patient is replaced or corrected by heterologous cells provided according to the present invention, or by autologous cells provided according to the present invention having the deficient gene replaced or repaired by genetic modification methods. Further, the transgenes, and vectors of the present invention may be delivered in vivo. Finally, proteins, including CRISPR/CAS9 related proteins, may be delivered by electroporation in vivo or in vitro, as taught herein. Examples of diseases amenable to such correction, replacement or repair include, but in no way are limited to, Canavan's disease (ASP); Tay-Sachs disease (HEXA); Lesch-Nyhan syndrome (HRPT); Huntington's disease(HTT); Sly syndrome; type A and type B Niemann Pick disease; Sandhoffs disease (HEXB); Fabry's disease (GLA); type C Niemann-Pick disease(NPC1); Gaucher's disease (GBA); Parkinson's disease(PARK2, etc.); Von Hippel Lindau's disease, Sickle cell anemia (HBB) and other thalassemias as well as similar diseases. Other exemplary diseases and disorders amenable to such correction, replacement or repair include diseases and disorders caused by mutations in the following genes: SNAP29, BCS1L, ALAS2, ESCO2, IDS, RP1, RP2, RPGR, PRPH2, IMPDH1, PRPF31, CRB1, PRPF8, TULP1, CA4, HPRPF3, ABCA4, EYS, CERKL, FSCN2, TOPORS, SNRNP200, PRCD, NR2E3, MERTK, USH2A, PROM1, KLHL7, CNGB1, TTC8, ARL6, DHDDS, BEST1, LRAT, SPARA7, CRX, NODAL, NKX2-5, ZIC3, CCDC11, CFC1, SESN1, CLCN5, OCRL, AASDHPPT, ATP7B, MEFV, ABCA1, MSTN, C2ORF37, FECH, FGFR2, IGF-2, CDKN1C, H19, KCNQ1OT1, BTD, BCS1L, FLCN, ATP2A1, NOTCH3, HPS1, HPS3, HPS4, HPS5, HPS6, HPS7, AP3B1, SCN1A, SCN2A, ABCC6, WT1, ZEB2, LMNA, PMP22, MFN2, PSEN1, PSEN2, APP, APOEÎμ4, ALAD, UBE3A, FGFR2, VPS33B, ATM, PITX2, FOXO1A, FOXC1, PAX6, DMPK, CNBP, FVIII, KRT5, KRT14, DSP, PKP1, JUP, PLEC1, DST, EXPH5, TGM5, LAMA3, LAMB3, LAMC2, COL17A1, ITGA6, ITGA4, ITGA3, COL7A1, FERMT1, MYO5A, RAB27A, MLPH, RYR1, TP53, NTRK1, GALNS, GLB1, AP4M1, AP4S1, AP4B1, AP4E1, PTEN, PDGFRB, BCKDHA, BCKDHB, DBT, DLD, GALT, GALK1, GALE, COL2A1, PMP22, APC, TCF4, STK11, MYO7A, USH1C, CDH23, PCDH15, USH1G, USH2A, GPR98, DFNB31, CLRN1, DHCR7, PROS1, ASPM, AGXT, GRHPR, DHDPSL, MEN1, RAG1, RAG2, GLA, LYST, PKD1, PKD2, HPRT, RELN, MMAA, MMAB, MMACHC, MMADHC, LMBRD1, MUT, FGFR3, DNAI1, DNAH5, TXNDC3, DNAH11, DNAI2, KTU, RSPH4A, RSPH9, LRRC50, UROD, TBX4, MSH2, MLH1, MSH6, PMS2, PMS1, TGFBR2, MLH3, RAB23, CREBBP, COL11A2, ATP2A2, RPS6KA3, CFTR, ATP7A, COL1A1, COL1A2, COL3A1, COL5A1, COL5A2, TNXB, ADAMTS2, PLOD1, B4GALT7, DSE, FBN1, MR1, CPDX, SGSH, NAGLU, HGSNAT, GNS, GLDC, AMT, GCSH, ERCC6, ERCC8, ABCA12, FGFR2, ASPA, CBS, GBA, PANK2, EXT1, EXT2, EXT3, DYSF, TIA1, GNE, MYH7, Titin, MYOT, MATR3, GCDH, ETFA, ETFB, ETFDH, SMPD1, NPA, NPB, NPC1, NPC2, 21, FGFR3, IDUA, MYCN, HSPG2, MECP2, PPDX, IKBKG, AAAS, FGD1, EDNRB, CP, LMBR1, COL2A1, FGFR3, HMBS, ADSL, GUSB, HDAC8, SMC1A, NIPBL, SMAS, RAD21, PC, TCOF1, POLR1C, or, POLR1D), HGD, COL4A3, COL4A4, COL4A5, ATP1A3, C9orf72, SOD1, FUS, TARDBP, CHCHD10, MAPT, ALMS1, ENG, ACVRL1, MADH4, HTT, ATXN1, ATXN2, ATXN3, PLEKHG4, SPTBN2, CACNA1A, ATXN7, ATXN8OS, ATXN10, TTBK2, PPP2R2B, KCNC3, PRKCG, ITPR1, TBP, KCND3, FGF14, CHD7, ABCD1, JAG1, NOTCH2, TP63, TREX1, RNASEH2A, RNASEH2B, RNASEH2C, SAMHD1, ADAR, IFIH1, GFAP, ARSB, PRNP, EMD, LMNA, SYNE1, SYNE2, FHL1, TMEM43, PDS, AASS, FGFR1, FGFR2, HTRA1, COL11A1, COL11A2, COL2A1, COL9A1, 11p15, FANCA, FANCB, FANCC, FANCD1, FANCD2, FANCE, FANCF, FANCG, FANCI, FANCJ, FANCL, FANCM, FANCN, FANCP, FANCS, RAD51C, XPF, RET, GCH1, PCBD1, PTS, QDPR, MTHFR, DHFR, IKBKAP, ATP13A2, MED12, PAX6, ATP2C1, GNE, MYHC2A, VCP, HNRPA2B1, HNRNPA1, COL1A1, COL1A2, IFITM5, COH1, ALDH3A2, FXN, AP1S1, PAX3, MITF, WS2B, WS2C, SNAI2, EDNRB, EDN3, SOX10, VHL, GALL, RAB3GAP, ABCB7, SLC25A38, GLRX5, 5q, HFE, HAMP, HFE2B, TFR2, TF, CP, HEXA, TSC1, TSC2, PAH, HYAL1, DMD, HEXB, ERCC, RUNX2, HSPB8, HSPB1, HSPB3, GARS, REEP1, IGHMBP2, SLC5A7, DCTN1, TRPV4, SIGMAR1, FGFR2, FGFR3, ABCA4, CNGB3, ELOVL4, PROM1, ALS2, PROC, INPP5E, TMEM216, AHI1, NPHP1, CEP290, TMEM67, RPGRIP1L, ARL13B, CC2D2A, OFD1, TMEM138, TCTN3, ZNF423, AMRC9, LCAT, COL11A1, COL11A2, COL2A1, PTPN11, KRAS, SOS1, RAF1, NRAS, HRAS, BRAF, SHOC2, MAP2K1, MAP2K2, CBL, PHF8, PEX1, PEX2, PEX3, PEX5, PEX6, PEX10, PEX12, PEX13, PEX14, PEX16, PEX19, PEX26. These transgenes (and/or their corresponding proteins) may represent the coding region or portions of the coding region of the normal genes. The nucleic acids (and/or their corresponding proteins) named herein may also be loaded into the cells of the present invention by various means (e.g electroporation) as a cellular cargo.

It is to be understood, however, that the scope of the present invention is not to be limited to the specific embodiments and examples described above. The invention may be practiced other than as particularly described and still be within the scope of the accompanying claims.

### Example 13

An Example Sequence for a Vector Capable of Rendering Cells Pluripotent and Expressing a Long Numb Isoform, Oct-4, Sox-2, and EmGFP Nucleic Acid Sequences (and/or their Corresponding Proteins) Under the Control of Tetracycline-Sensitive Promoters Corresponds to (SEQ ID NO: 2)

The vector may be constructed fully through de novo gene synthesis, or in part through the cloning of the Numb, Sox and OCT3/4 cDNA sequences (and/or their corresponding proteins) into the position occupied by LacZ in the Invitrogen pcDNA4tolacZ vector. Similarly, the tetR gene is found in the Invitrogen pcDNA6/TR vector. Coding sequences (and/or their corresponding proteins) of genes referenced are also appropriate for cloning into the pcDNA4lacZ vector.

Alternatively, the tetR gene may be transfected into target cells separately utilizing the pcDNA6/TR vector in combination with a vector comprising the sequence here minus the tetR gene and its PCMV promoter.

Likewise, multiple vectors may be employed so long as elements similar to the elements included in the above sequence are present. This may reduce the likelihood of promoter competition. It is to be understood that other conditional promoter elements may be substituted for the tetracycline sensitive promoter elements.

Integrase-deficient 2^{nd} and 3^{rd} generation lentiviral vectors may be utilized as non-integrating lentiviral vectors can be used as episomal vectors, in like manner to adenoviral (Lin et al., 2007), AAV, hybrid vectors, plasmid DNA, and other non-integrating vectors known to the art. Such integrase-deficient vectors can be readily introduced using a variety of standard transfection techniques (e.g. electroporation, chemically mediated transfection, fusogenic or non-fusogenic liposomes, lipofectamine, nanocapsules, nanovaults, etc.)—methods which allow high capacity integrase-deficient lentiviral vectors to be utilized without genomic integration and random alteration of the genome.

### Example 14

It is expected that intravenous and other administration of pluripotent stem cells produced according to the methods described herein (or other published methods) one or more times can provide replacement cells to the body and that such administration may serve to extend the life or improve the health of the patient suffering age-related senescence

### Example 15. Production of Germ Cells

The current invention covers the derivation of germ cells from dividing multipotent, pluripotent, totipotent, “VSEL-like” and/or “pluripotent-like” stem cells produced according to the methods described herein (or according to other published methods). The production of such germ cells may be suitable for treating infertility and producing embryos in vitro (e.g. Hubner et al., 2003; Kehler et al., 2005; Nayernia et al., 2006a; Nayernia et al., 2006b; Drusenheimer et al., 2007; Moore et al., 2007; etc.).

Likewise, the invention further covers transient or permanent transfection/contacting with other proteins and/or nucleic acid sequences, including ones selected from those encoding FIGLA, FIG alpha, DAZL, STRA8, FOXL2, OOGENESIN1, OOGENESIN2, OOGENESIN3, OOGENESIN4, SYCP2, SYCP3, SPO11, REC8, DMC1, MOS, STAG3, CCNB1, FOXO1, FOXO3, SOHLH1, SOHLH2, NOBOX, OBOX1, OBOX2, OBOX3, OBOX4, OBOX6, LHX8, LHX9, OOG1, SP1, ZFP38, TRF2, TB2/TRF3, TAF4B, TAF7L, TAF7l, TIA1, PHTF1, TNP2, HILS1, DAZL, BMP15, PTTG3, AURKC, OTX2, SOX15, SOX30, FOXR1, ALF, OCT4, DPPA3/STELLA, ZFP38, RPS6KA3, HINFP, NPAT, SP1, SP3, HOXA1, HOXA7, HEX, YP30, ZP1, ZP2, ZP3, SFE1, SFE9, OPO, PLN, RDV, GLD1, MMU-MiR351, MMU-MiR615, MMU-MiR592, MMU-MiR882, MMU-MiR185, MMU-MiR491, MMU-MiR326, MMU-MiR330, MMU-MiR351.

Likewise, the invention further covers transfection/contacting with other proteins and/or nucleic acid sequences, including ones selected from those encoding SYCP2, SYCP3, SPO11, REC8, DMC1, MOS, STAG3, OCT4, ALF, RPS6KA3, HINFP, SP1, SP3, TAF7l, TIA1, PHTF1, TNP2, HILS1, CLGN, TEKT1, FSCN3, DNAHC8, LDHC, ADAM3, OAZ3, AKAP3, MMU-MiR351, MMU-MiR615, MMU-MiR592, MMU-MiR882, and MMU-MiR185.

Likewise, the invention further covers transfection/contacting with other proteins and/or nucleic acid sequences, including ones selected from those encoding MOS, CCNB1, OCT4, FIG alpha, FIGL alpha, ALF, SOHLH1, SOHLH2, LHX8, LHX9, OOG1, FIG alpha, SP1, LHX3, LHX9, TBP2/TRF3, DAZL, BMP15, GDF9, PTTG3, AURKC, OTX2, SOX15, SOX30, FOXR1, NOBOX, OBOX1, OBOX2, OBOX3, OBOX6, OOGENESIN1, OOGENESIN2, OOGENESIN3, OOGENESIN4, YP30, ZP1, ZP2, ZP3, SFE1, SFE9, OPO, PLN RDV, GLD1, DAZL, STRA8, MMU-MiR615, MMU-MiR491, MMU-MiR326, MMU-MiR330, MiR212 and MMU-MiR351.

### Example 16: Generation of Transgenic Animals

The present invention covers the generation of transgenic animals. As with other pluripotent cells, the totipotent, pluripotent or pluripotent-like cells produced according to the methods described herein (or other published methods) may be utilized to produce transgenic animals by any method known to the art.

### Example 17: Therapeutic Vector Construction

Examples of retroviral vectors which may be employed include, but are not limited to, those derived from Moloney Murine Leukemia Virus, Moloney Murine Sarcoma Virus, and Rous Sarcoma Virus, FIV, and HIV. Appropriate expression vectors are that may be employed for transfecting DNA or RNA into eukaryotic cells. Such vectors include, but are not limited to, prokaryotic vectors such as, for example, bacterial vectors; eukaryotic vectors, such as, for example, yeast vectors and fungal vectors; and viral vectors, such as, but not limited to, lentiviral vectors, adenoviral (Lin et al., 2007) vectors, adeno-associated viral vectors, and retroviral vectors.

The replication incompetent pcDNA 6.2 GW/miR and pcDNA 6.2/EmGFP-Bsd/V5-DEST vectors are examples of an appropriate expression vectors (Invitrogen) and allow expression of synthetic oligonucleotides (e.g. miRNAs) that have the capacity to cleave targeted sequences (or their corresponding proteins). These vectors include flanking and loop sequences (and/or their corresponding proteins) from endogenous miRNA to direct the excision of the engineered miRNA from a longer Pol II transcript (pre-miRNA).

Alternatively, inclusion of the HIV psi sequence allows the therapeutic vector to compete with native HIV genome for packaging into viral particles, also inhibiting HIV transmission.

Combining multiple miRNA sequences (and/or their corresponding proteins) directed against a single target increases the likelihood of success in reducing target sequence expression. miRNA sequences (and/or their corresponding proteins) may be operably linked to tissue specific promoters such as the EF-1 alpha promoter, any T cell specific promoter, or macrophage specific promoter to ensure expression in the desired cell types.

Utilizing Invitrogen's lentiviral destination (DEST) vectors for gene expression, the resulting therapeutic vector(s) becomes capable of stably transducing both dividing and non-dividing cell types.

In a preferred embodiment, the therapeutic vector(s) contains multiple synthetic oligonucleotide sequences driven by one or more promoters so as to reduce expression of CXCR4, CCR5, and/or any other cellular protein known to act as a co-receptor for HIV infection in target cells.

In one therapeutic vector (constructed in 2006), four miRNA sequences (and/or their corresponding proteins) targeting CXCR4 and CCR5 co-receptors were cloned into the pcDNA 6.2 GW/miR vector along with decoy RNA sequences (and/or their corresponding proteins) targeting HIV-2 TAR and RRE.

Genetic constructs may include a vector backbone, and a transactivator which regulates a promoter operably linked to heterologous nucleic acid sequences (or their corresponding proteins).

Another example of a suitable vector is a retroviral vector. Retroviruses are RNA viruses which contain an RNA genome. The gag, pol, and env genes are flanked by long terminal repeat (LTR) sequences (or their corresponding proteins). The 5′ and 3′ LTR sequences (and/or their corresponding proteins) promote transcription and polyadenylation of mRNAs.

The retroviral vector may provide a regulable transactivating element, an internal ribosome reentry site (IRES), a selection marker, and a target heterologous gene operated by a regulable promoter.

Alternatively, multiple sequences (and/or their corresponding proteins) may be expressed under the control of multiple promoters. Finally, the retroviral vector may contain cis-acting sequences (and/or their corresponding proteins) necessary for reverse transcription and integration. Upon infection, the RNA is reverse transcribed to DNA which integrates efficiently into the host genome. The recombinant retrovirus of this invention is genetically modified in such a way that some of the retroviral, infectious genes of the native virus are removed and in embodiments replaced instead with a target nucleic acid sequence for genetic modification of the cell. The sequences (and/or their corresponding proteins) may be exogenous DNA or RNA, in its natural or altered form.

### Example 18: Example Methods for Generation of the Therapeutic Vector

The methods for generation of the therapeutic vector(s) include those taught in Invitrogen's Viral Power Lentiviral Expression Systems Manual (incorporated by reference herein). Briefly, the EmGFP-bsd cassette is cloned as a PmlI-BlpI fragment into the pLenti6/R4R2/V5-DEST vector, while the miR-decoy cassette is simultaneously transferred by BP reaction into pDONR221. Then the EF1a promoter and miR-decoy are Multi-site LR crossed into the modified pLenti6/EmGFP-bsd/R4R2-DES Tvector.

pLenti6/R4R2/V5-DEST vector sequence (SEQ ID NO: 1), Example miR-decoy cassette sequence (SEQ ID NO: 3).

### Example 19: Methods for Propagating/Proliferating Stem/Progenitor Cells In Vivo

In order to obtain large numbers of target cells that are relatively resistant to 1) HIV infection and/or 2) HIV replication and/or 3) HIV transcription, progenitor/stem cells can be grown in Dulbecco's modified Minimal Essential Medium (DMEM) supplemented with glutamine, beta.-mercaptoethanol, 10% (by volume) horse serum, and human recombinant Leukemia Inhibitory Factor (LIF). The LIF replaces the need for maintaining progenitor/stem cells on feeder layers of cells, (which may also be employed) and is essential for maintaining progenitor/stem cells in an undifferentiated state.

### Example 20

Cells are collected from individuals, developmentally-activated, transfected with the therapeutic vectors, then cultured and prepared for autologous or heterologous transplantation by standard methods, with or without HLA typing and matching.

### Example 21

Umbilical cord blood samples are obtained from umbilical blood cord bank. The cells are then (with or without developmental activation) transfected with the therapeutic vector of beneficial sequences (or their corresponding proteins), then prepared for transplantation by standard methods, with or without HLA typing and matching.

### Example 22: Examples of Synthetic Oligonucleotide Sequences Suitable for Inclusion in the Therapeutic Vector

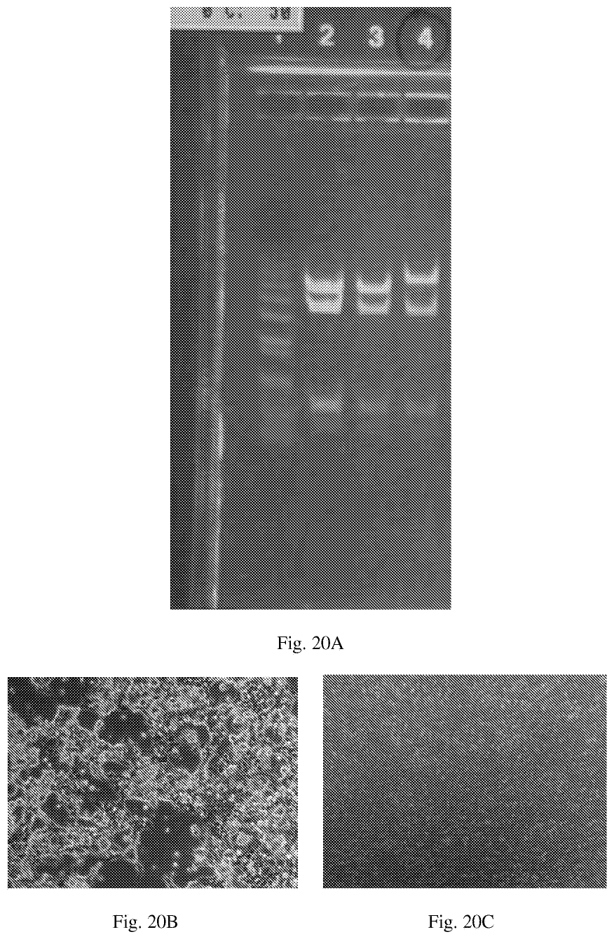

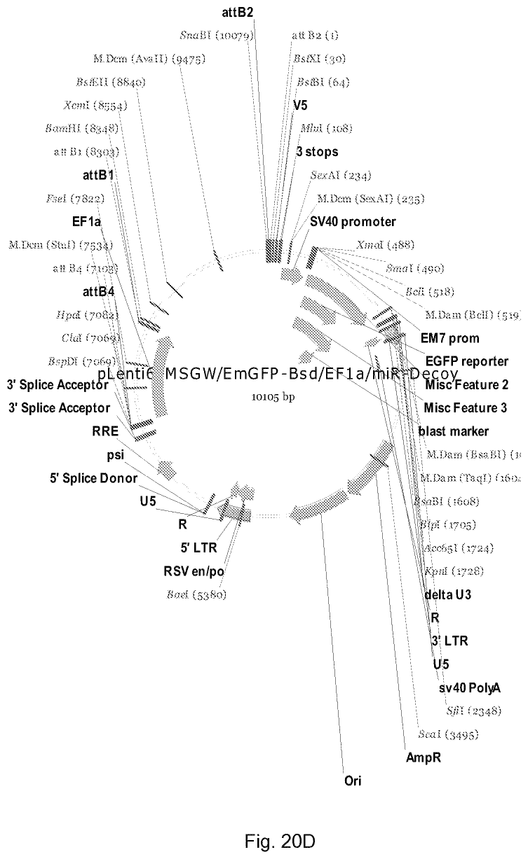

Any synthetic oligonucleotide sequences that successfully reduce the protein expression of targeted sequences (and/or their corresponding proteins) >70% is covered by the present invention. See FIG. 20D.

Any synthetic oligonucleotide sequences that successfully reduce the ability of target cells to sustain HIV replication by >70% or to a lesser but therapeutic degree or HIV viral activity by >70% or to a lesser but therapeutic degree are also covered by this invention.

Examples of miRNA sequences include miRNA sequences derived by IVGN algorithm (Invitrogen). miRNA sequences targeting the CXCR4 gene include top strand: 5′-TGCTGATACCAGGCAGGATAAGGCCAGTTTTGGCCACTGACTGACTGGCCTTACTGC CTGGTAT-3′ (SEQ ID NO: 4) and bottom strand: 5′-CCTGATACCAGGCAGTAAGGCCAGTCAGTCAGTGGCCAAAACTGGCCTTATCCTGCC TGGTATC-3′ (SEQ ID NO: 5); as well as top strand: 5′-TGCTGTGACCAGGATGACCAATCCATGTTTTGGCCACTGACTGACATGGATTGCATC CTGGTCA-3′ (SEQ ID NO: 6) and bottom strand: 5′-CCTGTGACCAGGATGCAATCCATGTCAGTCAGTGGCCAAAACATGGATTGGTCATCC TGGTCAC-3′ (SEQ ID NO: 7).

Similarly, miRNA sequences targeting the CCR5 gene include top strand: 5′-TGCTGATCGGGTGTAAACTGAGCTTGGTTTTGGCCACTGACTGACCAAGCTCATTAC ACCCGAT-3′ (SEQ ID NO: 8) and bottom strand: 5′-CCTGATCGGGTGTAATGAGCTTGGTCAGTCAGTGGCCAAAACCAAGCTCAGTTTACA CCCGATC-3′ (SEQ ID NO: 9); as well as top strand5′-TGCTGATAGCTTGGTCCAACCTGTTAGTTTTGGCCACTGACTGACTAACAGGTGACC AAGCTAT-3′ (SEQ ID NO: 10) and bottom strand: 5′-CCTGATAGCTTGGTCACCTGTTAGTCAGTCAGTGGCCAAAACTAACAGGTTGGACCA AGCTATC-3′ (SEQ ID NO: 11).

### Example 23

Examples of Decoy RNA suitable for inclusion in the therapeutic vector. Any decoy sequences that successfully reduce the ability of target cells to sustain HIV replication by >70% or to a lesser but therapeutic degree or HIV viral activity by >70% or to a lesser but therapeutic degree are covered by this invention.

An example TAR decoy sequence is (SEQ ID NO: 12)

gtcgctctgcggagaggctggcagattgagccctgggaggttctctccag
cactagcaggtagagcctgggtgttccctgctagactctcaccagtgctt
ggccggcactgggcagacggctccacgcttgcttgcttaaagacctctta
ataaagctgc (Browning et al., 1999). See FIG. 20D.

An example RRE decoy sequence is (SEQ ID NO: 13)

tgctagggttcttgggttttctcgcaacagcaggttctgcaatgggcgcg
gcgtccctgaccgtgtcggctcagtcccggactttactggccgggatagt
gcagcaacagcaacagctgttggacgtggtcaagagacaacaagaactgt
tgcgactgaccgtctggggaacgaaaaacctccaggcaagagtcactgct
atagagaagtacctacaggaccaggcgcggctaaattcatggggatg
(Dillon et al., 1990). See FIG. 20D.

### Example 24: Flanking Sequences Providing Stability for RNA Decoys

Examples of appropriate flanking sequences for RNA decoys are as follows:

(SEQ ID NO: 14)
GUGCUCGCUUCGGCAGCACGTCGAC ---TAR DECOY SEQ---
(SEQ ID NO: 15)
UCUAGAGCGGACUUCGGUCCGCUUUU
(SEQ ID NO: 16)
GUGCUCGCUUCGGCAGCACGTCGAC ---RRE DECOY SEQ---
(SEQ ID NO: 17)
UCUAGAGCGGACUUCGGUCCGCUUUU. See FIG. 20D.

Previously, it was demonstrated that decoy sequences flanked by hairpins on either side, 19 nucleotides (ntds) of the U6 RNA on the 5′ side as well as a 3′ stem immediately preceding a poly U terminator for POLIII, showed greater stability. This arrangement is expected to protect against 3′-5′ exonuclease attack, and to reduce the chances of the 3′ trailer interfering with the insert RNA folding. Since only the first 3/4 of the tRNA sequence is present, the 5′ end of the insert should be protected and export from the nucleus should be prevented (Good et al., 1997).

### Example 25: Introduction of Therapeutic Vector to the Host

In a preferred embodiment, blood stem/progenitor cells, and target cells are transfected with the therapeutic vector(s) (or associated therapeutic virus) in vivo by introduction of the therapeutic vector(s) into the host blood, tissues, or bone marrow, etc. The greatest benefit may be achieved by modifying a large number of endogenous target and stem/progenitor cells. This may be accomplished by using an appropriately-sized, catheter-like device, or needle to inject the therapeutic vector(s) into the venous or arterial circulation. In a preferred embodiment, the virus is pseudotyped with VSV-G envelope glycoprotein and native HIV-1 env proteins.

### Example 26: Introduction of Genetically-Modified Cells into the Host

Blood cells, such as mature peripheral blood T lymphocytes, monocytes, macrophages, T cell progenitors, macrophage-monocyte progenitor cells, and/or pluripotent hematopoietic stem cells (such as those found in umbilical cord blood and occupying bone marrow spaces) as well as other stem/progenitor cells can be transfected using the therapeutic vector(s) in vitro. Appropriate concentrations of the therapeutic vector(s) may be those consistent with Browning et al., 1999. Subsequently, cells are expanded (propagated) in vitro, and are then transferred to the host via introduction of the cells to the venous or arterial circulation using an intravenous needle or catheter. Subsequently, cells transfected with the therapeutic vectors are able to “home” to the bone marrow and other tissues.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### Example 27

Fluorescein-conjugated albumin protein is delivered in high concentration to the interior of 3T3 cells with high efficiency using protein electroporation, according to the method of Koken et al., 1994.

### Electroporation and MagnetoPoration

Application of an electric field to Cells via Electroporation and Magnetic Field generation (aka Magnetoporation) showed comparable performance in driving uptake of exogenous markers. Cells electroporated at 300V with a varying number of 5 ms pulses showed progressively increased protein uptake (see FIGS. 9A-9H). ˜200,000 cells were electroporated in a 4 mm cuvette in the presence of FITC-conjugated albumin (150 ug/200 ul). Visual inspection and photography using a fluorescent microscope revealed progressively increased FITC-albumin uptake and fluorescence over a wide range of pulse number (10-200 pulses). FIG. 9 depicts the cells (at low power magnification, 10×) 48 hours after exposure to 20 pulse (FIG. 9A), 50 pulse (FIG. 9C), 100 pulse (FIG. 9E) and 200 pulse (FIG. 9G) electroporation, demonstrating protein uptake was a function of electroporation. Corresponding fluorescence intensity histograms are depicted in FIGS. 9B, 9D, 9F, and 9H. Similarly, a cell line exposed to an electric field (200 or 300 pulses), showed extensive uptake of EtBr (Novickij et al., 2013). Meanwhile, Magnetoporation has two major advantages over electroporation: pulse effectiveness is independent of the medium and not limited by electrode composition.

### Example 28

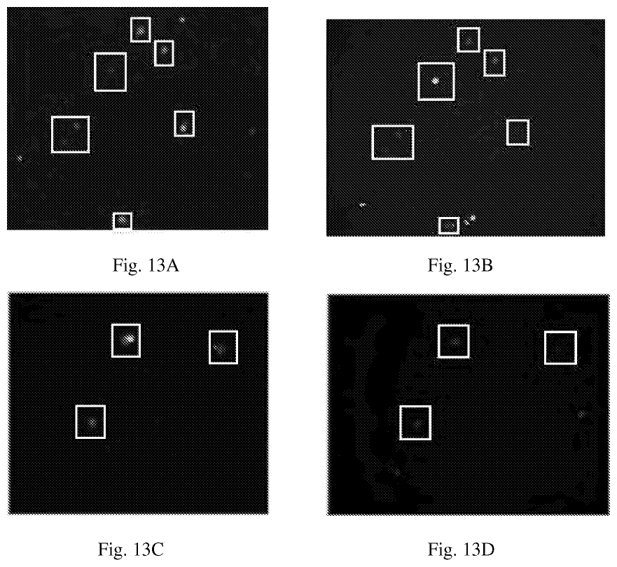

Long PRR+Numb alone, or in combination with Oct4, Sox2, Nanog and/or Notch proteins, activated the cells such that they were shown to be newly-positive for SSEA3, SSEA4, and Tra-1-81 antigens, indicative of pluripotent stem cells, as well as endogenous Oct4, Sox2, Nanog and PRR+ Numb proteins (FIGS. 13 and 14).

### Cell Culture

Prior to electroporation, 3T3 cells and 3T3-PRR+ Numb cells were maintained in growth medium (GM) containing DMEM supplemented with 10% fetal bovine serum (FBS), 20 mM L-glutamine, and 1% penicillin/streptomycin.

### Protein Electroporation

3T3 cells and 3T3-PRR+ Numb cells were harvested and electroporated using the BTX ECM 830 electroporation machine according to the methods of Koken et al., 1994. Briefly, ˜200,000 cells were transferred to 4 mm cuvettes and electroporated in the presence of either 50 ug Notch protein or 150 ug of oct4/sox2/nanog protein cocktail. Immediately following electroporation, the treated cells were transferred to Dulbecco's Modified Eagle Medium (DMEM) in standard cell culture plates, with or without 20 ng/ml EGF, and incubated at 37 degrees Celsius.

### Immunohistochemistry

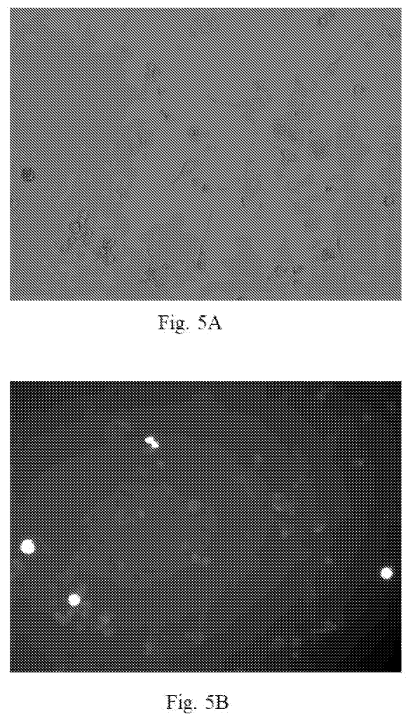

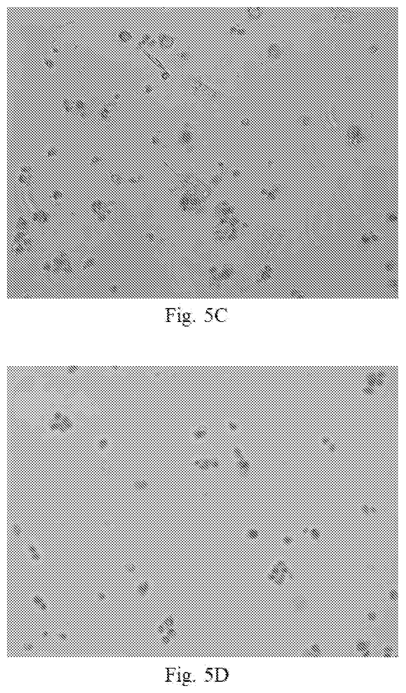

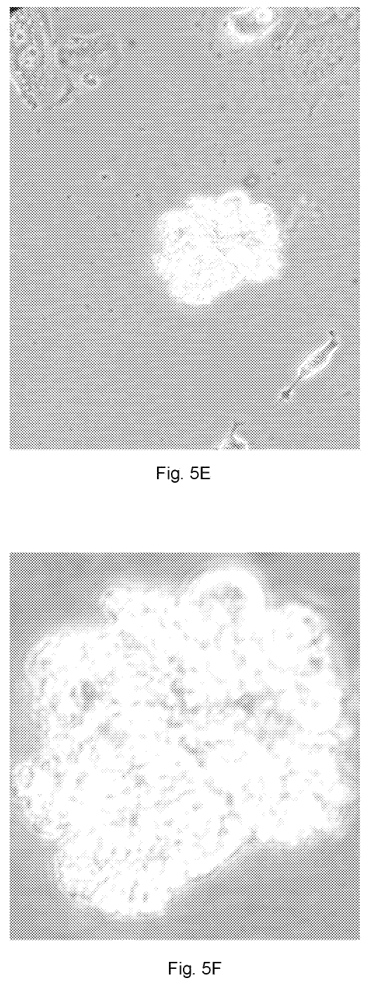

In all of these experimental conditions, long PRR+ Numb alone or in combination with Oct4, Sox2, Nanog and/or Notch proteins activated the cells such that they were shown to be newly-positive for SSEA3, SSEA4, and Tra-1-81 antigens, indicative of pluripotent stem cells, as well as endogenous Oct4, Sox2, Nanog and PRR+ Numb proteins. See FIGS. 5A-5D and 6A-6D.

### Example 29

Non-pluripotent murine cells electroporated in the absence of reprogramming factors, or in the presence of Oct4, Sox2, Nanog and Notch-1 proteins (300V, 70 pulses, 50 ug per protein), or overexpressing the long PRR+Numb isoform, cluster hierarchically and in Principal Component Analysis (PCA) plots amongst published, pluripotent ES and iPS cells.

### Cell Culture

Prior to electroporation, murine cells and PRR+Numb overexpressing murine cells were maintained in growth medium (GM) containing DMEM supplemented with 10% fetal bovine serum (FBS), 20 mM L-glutamine, and 1% penicillin/streptomycin.

### Protein Electroporation

Starting cells were harvested and electroporated using the BTX ECM 830 electroporation apparatus according to the methods of Koken et al., 1994. Briefly, 200,000 cells were transferred to 4 mm cuvettes and electroporated in the presence of 200 ug/ml oct4/sox2/nanog/notch protein cocktail. Immediately following electroporation, the cells were a) returned to growth medium at 37 degrees.

Resulting embryoid bodies, floating colonies, and cells adherent 4 days post-electroporation were collected and their RNA extracted for gene array analysis using the Affymetrix GeneChip HTA 2.0 chip. Murine Cells electroporated with Oct4, Sox2, Nanog and Notch-1 proteins (300V, 70 pulses, 50 ug per protein), or overexpressing the long PRR+Numb isoform, clustered hierarchically in Heatmaps and in Principal Component Analysis (PCA) plots amongst published, pluripotent ES and iPS cells (GSE53299 and GSE61403).

Reactome analysis (https://reactome.org/PathwayBrowser) was further performed based on the results of the gene array analysis and revealed that treated cells showed enriched or overexpressed genes in, for example the Cell Cycle pathways and Developmental pathways (including the Transcriptional regulation of pluripotent stem cells sub-pathway).

### Example 30

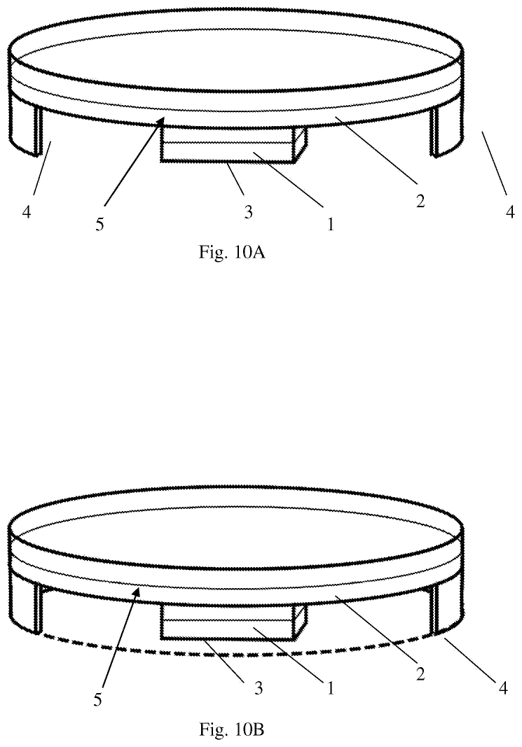

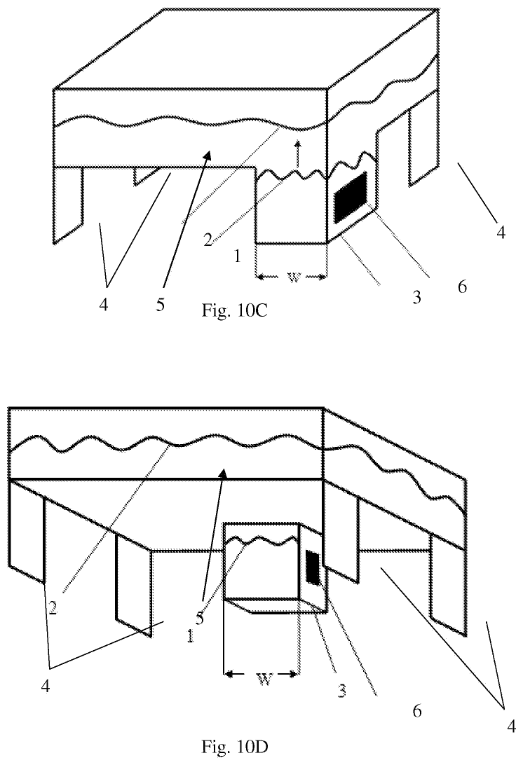

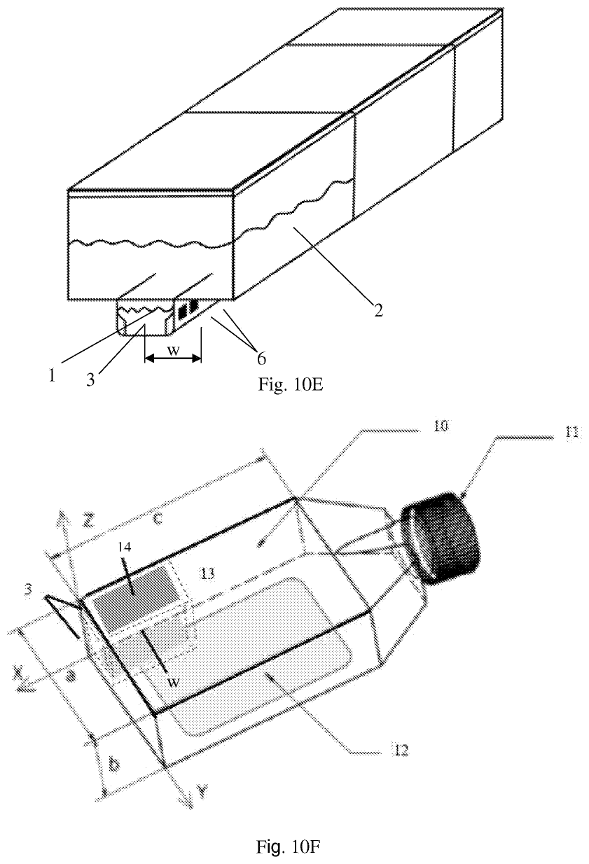

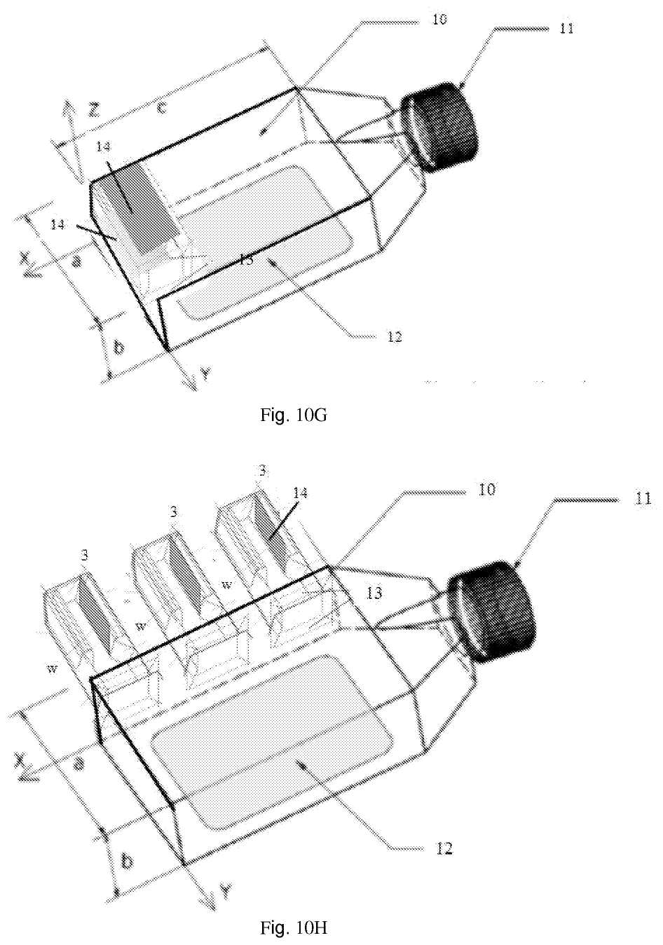

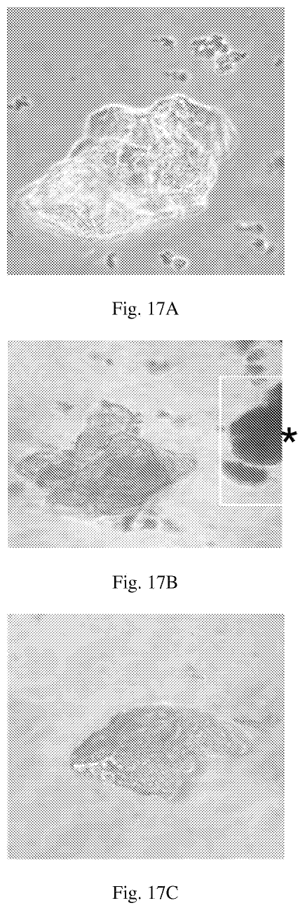

After electroporation with Oct4, Sox2, and Nanog proteins, human buccal cheek cells are induced to pluripotent-like, Developmentally-Activated Cells: They Divide, Form Colonies, Form Embryoid composed of very small cells, and Express Oct4, Sox2, and Nanog Proteins. ˜200,000 human buccal cheek cells were electroporated in a 4 mm cuvette with 300V in the presence of FITC-conjugated albumin (150 ug/200 ul). Approximately 200,000 human buccal cheek cells were electroporated in a 4 mm cuvette in the presence of FITC-conjugated albumin (150 ug/200 ul). Increasing number of pulses led to progressively increased FITC-albumin uptake and fluorescence. Photomicrographs show a small colony (FIG. 9A) and a much larger colony of proliferating epithelioid cells (FIG. 9B) induced 6 days after electroporation in the presence of 150 ug Oct4, Sox2 and Nanog protein. By 46 days post-electroporation with Oct4, Sox2, and Nanog proteins, colonies visible at low power (10×) mostly comprised darker appearing embryoid (asterisk) composed of VSEL-like cells (FIG. 9C). Immunohistochemistry showed electroporated cheek cells expressed Oct4 (FIG. 17A), Nanog (FIG. 17B), and Sox2 (FIG. 17C). Dense, darkly stained embryoid was composed of very small or VSEL-like cells (*) are apparent on FIG. 10B.

### Example 31

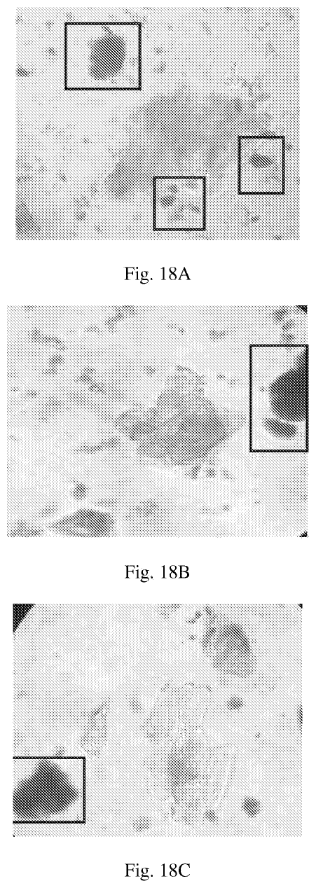

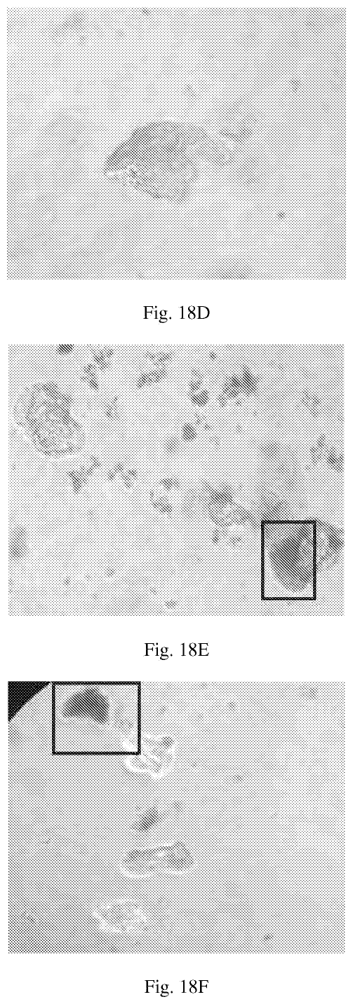

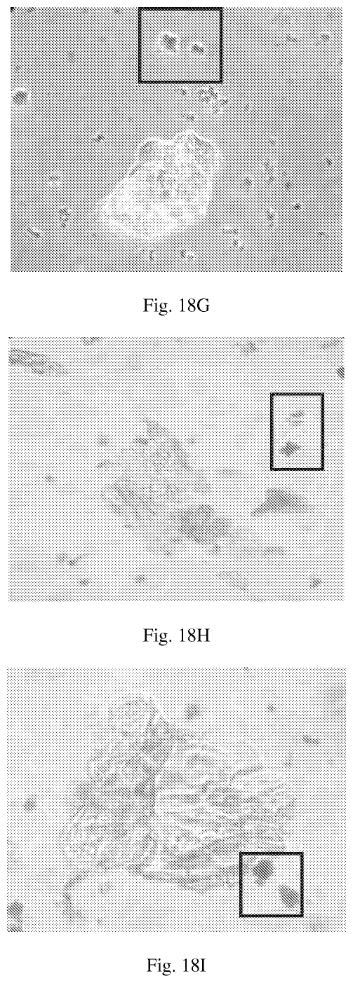

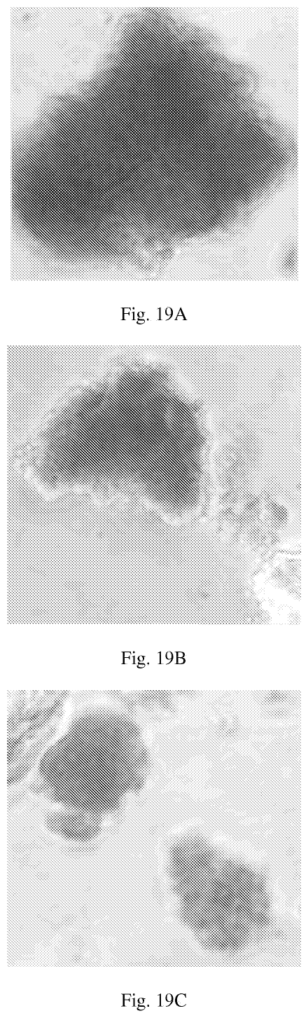

Electroporation “flips the Switch”. After electroporation in PBS with 50 ug, each, of Oct4, Sox2, and Nanog proteins, human buccal cheek cells are induced to pluripotent-like, Developmentally-Activated Cells (DAdC). 14 days post-electroporation, treated cells show altered morphologies, divide, form colonies, form embryoid composed of VSEL cells, and express Oct4, Sox2, and Nanog Proteins. (FIGS. 18A-18I). Embryoid positively-stained after immunohistochemistry using antibodies against Nanog, Sox2 and Oct4, well-known markers of pluripotency. Following electroporation in PBS (300V, 70 pulses), embryoid was formed by treated cells and expressed Oct4 (FIG. 19C), Sox2 (FIG. 19B) and Nanog (FIG. 19A) proteins and consists of VSEL-like cells (Ratajczak, et al., 2008; Kuruca, et al., 2019).

Reactome analysis (https://reactome.org/PathwayBrowser) was further performed based on the results of the gene array analysis and revealed that treated cells showed enriched or overexpressed genes in, for example the Cell Cycle pathways and Developmental pathways (including the Transcriptional regulation of pluripotent stem cells sub-pathway).

### Example 33

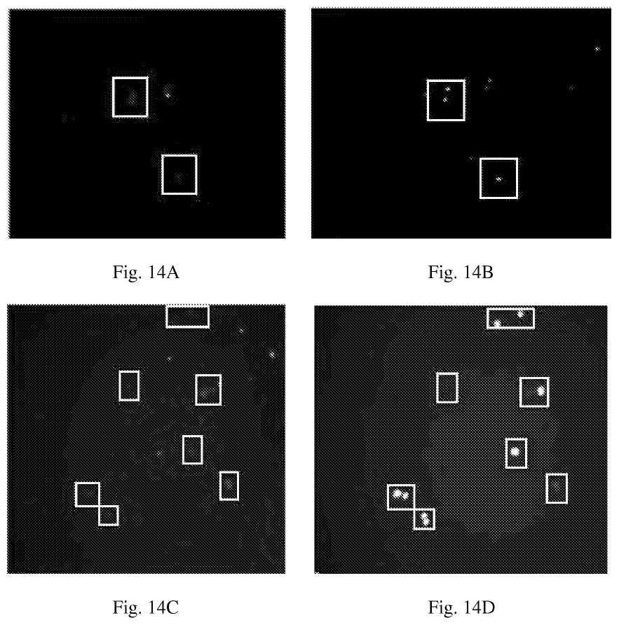

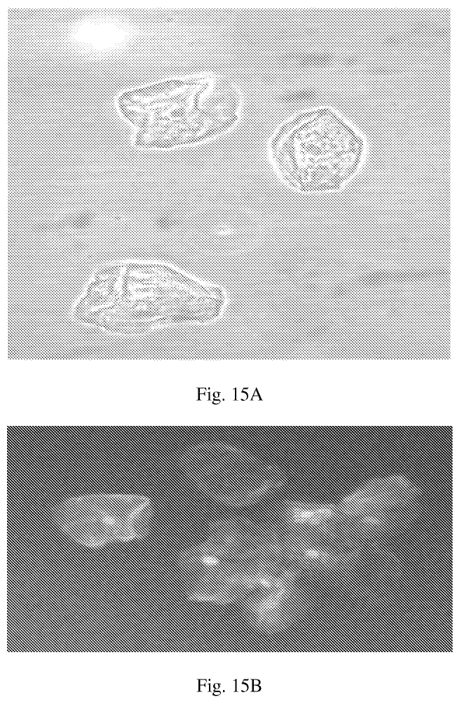

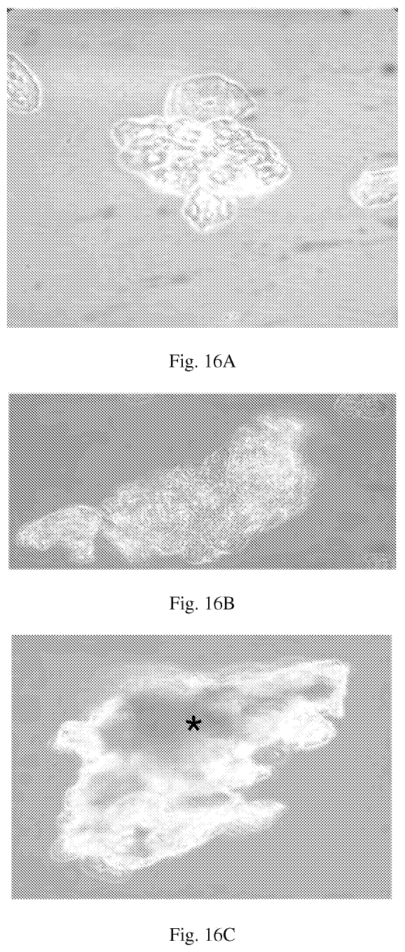

Protein electroporation according to the Method of Koken et al., 1994 (300V) provides delivery of protein at high concentrations to the interior of 3T3 cells for rapid (24-72 hr), efficient (100%) and durable (>60 days). Seventy, 300V pulses were delivered for 5 ms at 100 ms intervals. (FIGS. 14A-14D, FIGS. 15A-15D, FIGS. 16A-16D).

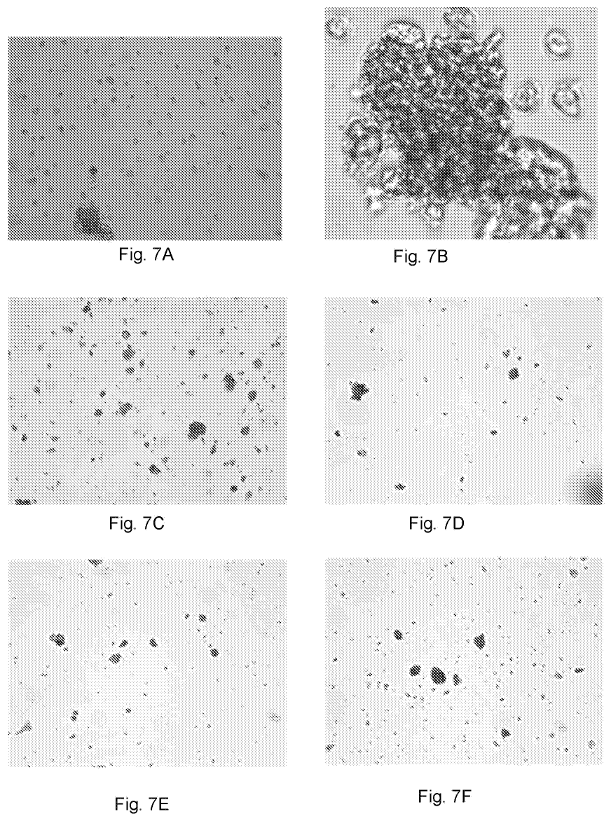

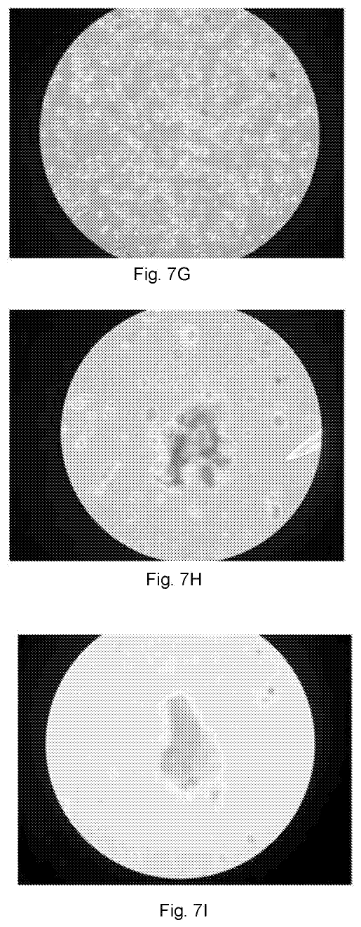

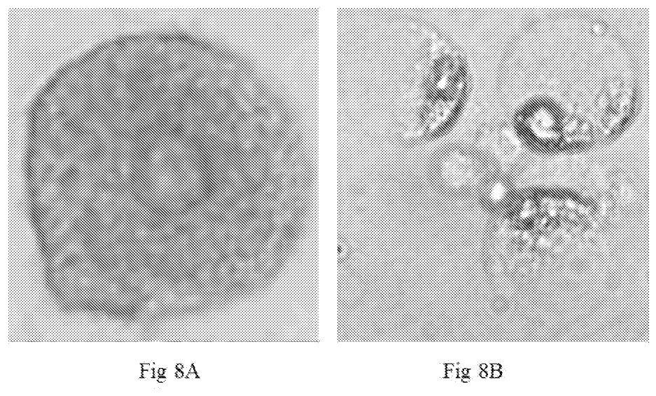

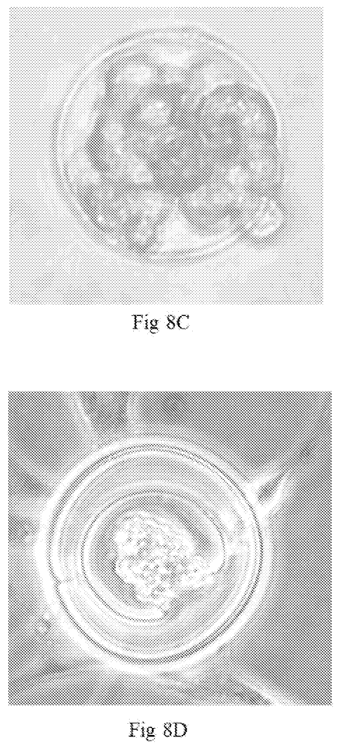

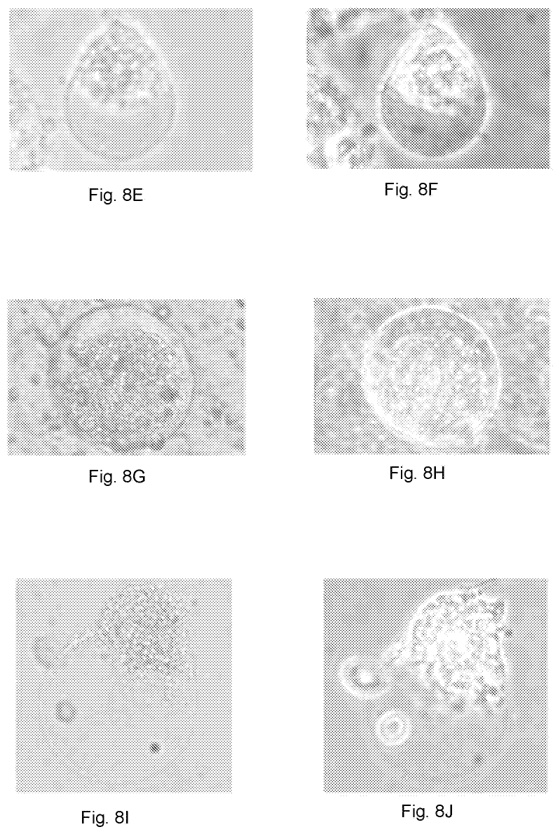

Less than 24 hours after protein electroporation with Oct4, Sox2, and Nanog proteins, approximately three percent of cells showed pluripotency induction (GFP reporter expression under the control of the c-MYC promoter). In contrast, 72 hours after protein electroporation (FIGS. 5A, 5B), >95% cells show pluripotency induction (c-MYC/GFP stem reporter expression (ABM)). Thirty days after Oct4/Sox2/Nanog Protein Electroporation, the resulting Embryoid bodies showed positive reactivity with anti-Oct4 (FIG. 7C), anti-Nanog (FIG. 7D), anti-Numb (FIG. 7E) and anti-Notch (FIG. 7F) antibodies.

After electroporation alone, or with Oct4, Sox2 and Nanog proteins (50 ug each) in PBS with seventy 5 ms pulses at 300V, treated cells were activated to adopted rounded, stem cell morphologies and formed small colonies on day six (FIGS. 5C and 5D) and early embryoid on day 9 (FIGS. 6A and 6B). Cells electroporated with Oct4, Sox2 and Nanog formed large embryoid bodies on day 40 (FIG. 7G and FIG. 7H), and large rafts of embryoid by day 57 (FIG. 7I).

### Example 34

Construction of the pLenti6-MSGW/EmGFP-Bsd/EF1a/miR-decoy HIV Gene Therapy Vector. Subcloning of the EmGFP-Bsd cassette from pcDNA™ 6.2/EmGFP-Bsd/V5-GW/CAT into the final vector was confirmed by Restriction Digestion (FIG. 20A). The pLenti6-MSGW/EmGFP-Bsd/EF1a/miR-decoy vector comprises HIV RRE and TAR decoy sequences, miRNA sequences directed against HIV co-receptors, CCR5 and CXCR4 and the HIV-2 psi sequence, all of which confer resistance to various human and animal immunodeficiency viruses. Virus stock was prepared from transfected 293FT cells. Successful transfection was confirmed by visualizing syncitia formation at 72 hours (FIG. 20B) versus control (FIG. 20C).

### Example 35

Combination of Long PRR+Numb transfection with Notch and/or Oct4/Sox2/Nanog Protein transfection (per Koken et al., 1994) produces the claimed effect (FIGS. 18A-18F, 19).

### Protein Electroporation

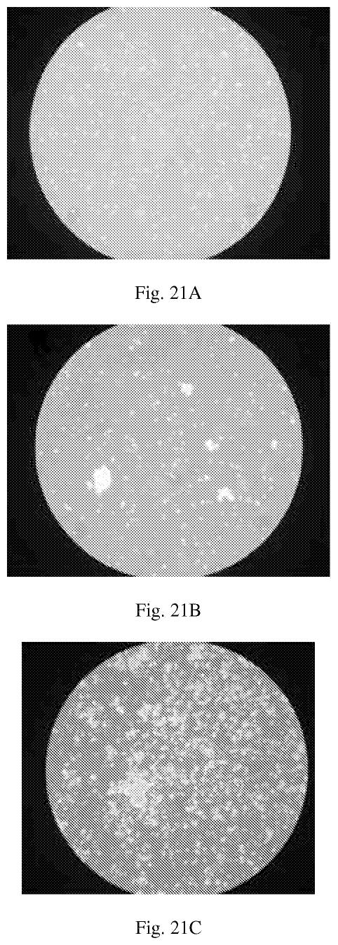

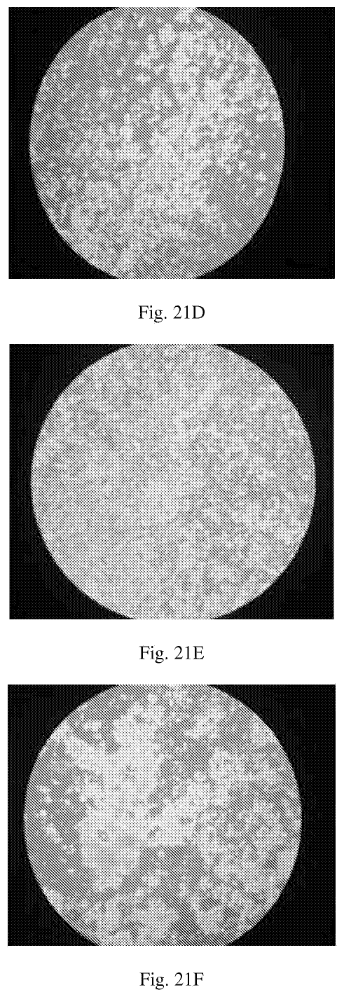

Equal numbers (˜200,000) of fibroblast cells and PRR+Numb overexpressing fibroblast cells were electroporated with: i) Notch protein alone; ii) Oct4, Sox2, and Nanog proteins; or iii) Notch, Oct4, Sox2, and Nanog proteins. On day thirty (30), non-adherent and floating cell colonies were collected and resuspended in equal volumes of medium for low power (10×) visual comparison of efficiencies: Control (FIG. 21A), Numb (FIG. 21B), Numb/Oct4/Sox2/Nanog (FIG. 21C), Numb/Notch (FIG. 21D), Numb/Notch/Oct4/Sox2/Nanog (FIG. 21E), and Notch/Oct4/Sox2/Nanog (FIG. 21F). In all of the experimental conditions, Oct4, Sox2, Nanog and/or Notch proteins (alone or in combination with transfected PRR+Numb) activated the cells such that they were shown to be newly-positive for SSEA3, SSEA4, and Tra-1-81 antigens, indicative of pluripotent stem cells, as well as endogenous Oct4, Sox2, Nanog and PRR+Numb proteins.

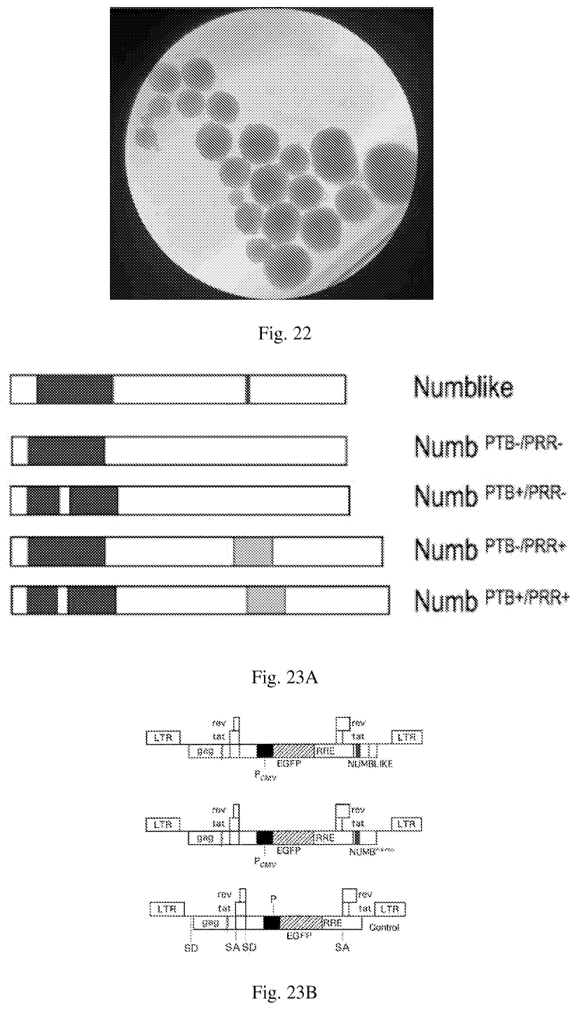

FIG. 22 illustrates an embryoid bodies 35 days post electroporation. A single round of electroporation according to the method of Koken et al. (1994), in the presence of Oct4, Sox2, Nanog, and Notch proteins (50 ug each) (Abcam), consistently activated cells with high efficiency (˜100% in some experiments) to form colonies, embryoid, and VSEL-like cells, consistent with pluripotency or a “pluripotent-like” state.

### Example 36

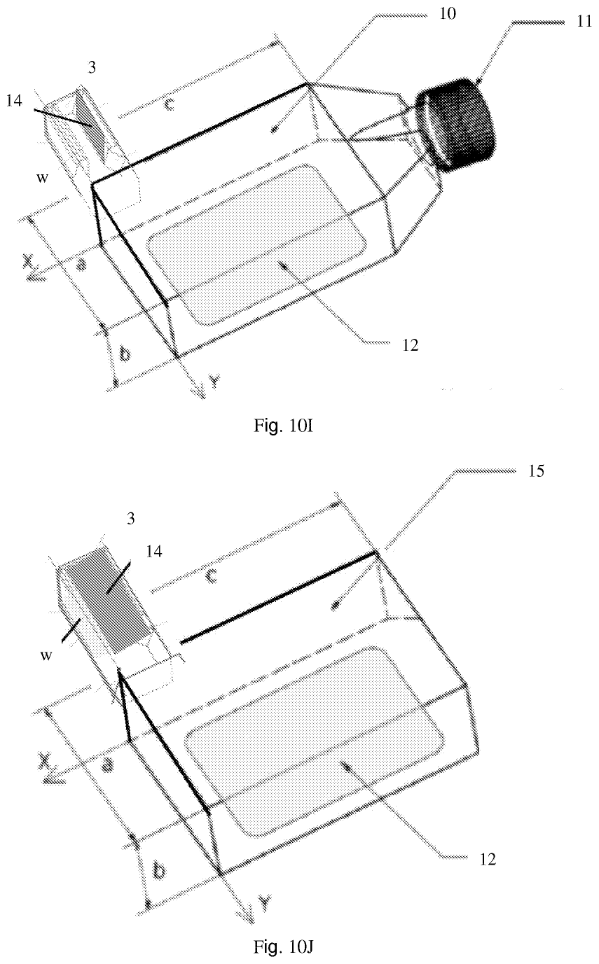

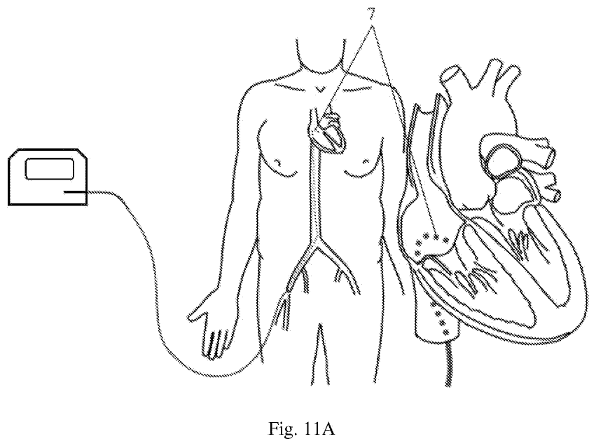

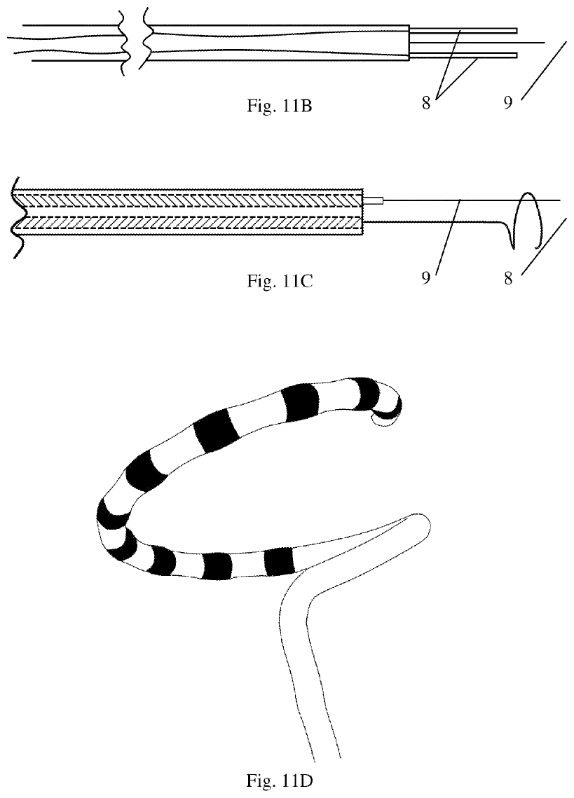

A catheter style electroporation/magnetoporation apparatus suitable for in vivo electroporation or magnetoporation alone or with protein and other transfectants consisting of a needle through which a protein, nucleic acid, drug, other transfectant, or cell population may be administered to a tissue (FIG. 11A).

This apparatus may include some variants of the catheter 7 (FIG. 11B and FIG. 11C). FIG. 11A shows the variant of catheter style electroporation/magnetoporation apparatus with variant of the electroporation/magnetoporation catheter 7 with needle 9 and two electrodes 8 (see FIG. 11B). FIG. 11D illustrates a loop or circular electrode 8 array which is used in catheter 7 (shown on FIG. 11C) suitable for in vivo electroporation. When magnetoporation is desired, the catheter features a magnetic coil assembly in place of electrodes.

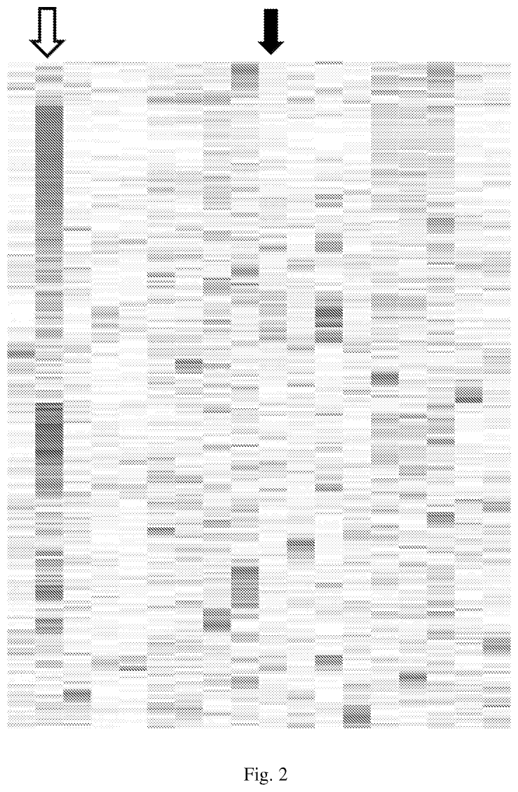

The needle 9 is i) situated between two electrode 8 prongs (which may be either sharp or dull) as shown in FIG. 2B, or the needle is ii) accompanied by a single internal electrode 8 (FIG. 2C) that is used in conjunction with an external electrode (as commonly occurs with cardiac ablation), or the needle doubles as a first electrode, iiia. and a second electrode is located alongside it, or iiib. a second electrode is located externally, or iiic. a second electrode is connected to a separate accompanying catheter. The separate accompanying catheter may also comprise a needle for delivering transfectant that doubles as a second electrode. The setup overall is akin to the setup uses for cardiac ablation, except that voltage is applied locally for cellular permeabilization and uptake of the transfectant.

FIG. 10 illustrates an assembly of sample cell culture dishes suitable for developmentally activating cells (number of dishes may vary for example from 1 to 1,600) wherein each has a “reservoir” 3 portion that functions like a typical electroporation cuvette and broader portion (“inspection plane”) 5 which functions like a traditional cell culture plate. An assembly may feature one or more plate covers. The dimensions (including width) of the reservoir 3 may vary and approximate the dimensions and materials of traditional electroporation cuvettes allowing for example, for 1 mm, 2 mm, 4 mm, 6 mm gaps, etc. In some embodiments, the electrode contact 6 is visible along the side of the reservoir 3. “w” indicates width of reservoir 3 of dish.

The reservoirs 3 and inspection planes 5 may take various shapes (FIGS. 10A-10D) and be positioned centrically or eccentrically relative to one another. FIGS. 10A, 10C and 10D show additional “feet” 4 along the edge of the plates that may or may not be detachable, so that the dishes will be able to stand on a flat surface. FIG. 10B shows “skirt” 4 along the edge of the plates that may or may not be detachable, so that the dishes will be able to stand on a flat surface. The various dishes may feature detachable base/stand with foot processes or skirts to provide stability. Alternatively, the dish may be manufactured with base/stand incorporated with the dish as a single piece.

Electroporation will typically occur in the reservoir 3. Additional media may be added before or after electroporation allowing cells to be incubated in larger volumes of media than are accommodated by the reservoir alone.

The volume of media contained in the reservoir portion is designated 1. The volume of media contained in cell culture dish overall is designated 2.

### Example 37

Structural comparison between mouse Numblike and its mammalian Numb homologues and construction of integrase-deficient, transgene expressing lentivectors.

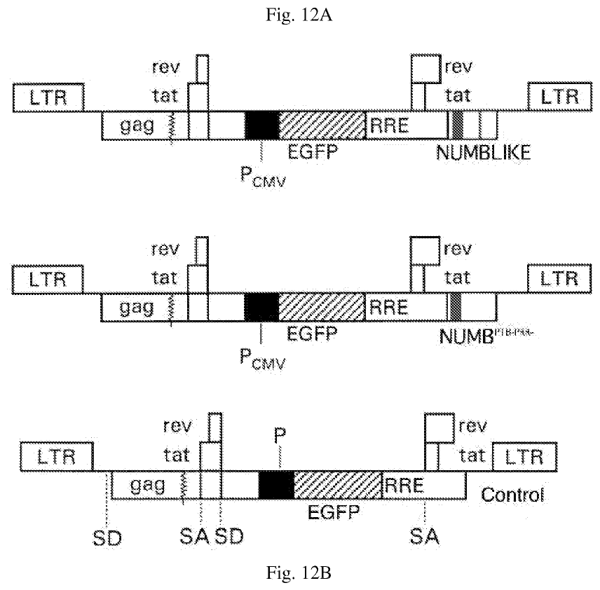

FIG. 12A illustrates that Numblike shows greater than 70% sequence identity in its amino terminal half to the shortest Numb homologue, but less than 50% identity in its cytoplasmic half where a unique 15 amino acid polyglutamine domain (purple) is found. The longest Numb isoform contains an 11 amino acid insert (white) within its phosphotyrosine binding (PTB) domain (black), as well as a 49 amino acid insert (gray) adjacent to a proline rich region (PRR). Two intermediate sized isoforms contain either the PTB or PRR inserts, but not both. The shortest Numb isoform lacks both inserts. FIG. 12B illustrates the HIV-EGFP Numblike and HIV-EGFP-NumbPTB+/PRR+vectors constructed from the two-gene HIV-EGFP-HSA vector (Reiser et al., 2000) by cloning the transgene cDNAs into nef coding region previously occupied by the mouse HSA cDNA. Abbreviations: Rev-response element (RRE), slice donor site (SD), splice acceptor site (SA).

### Example 38

In vivo injection of the HIV-EGFP-Numblike transfectant into the lateral ventricle and subsequent electroporation.

a) 72 hours after transient transfection, pairs and clusters of EGFP-positive cells were detected migrating radially in the mouse forebrain. FIG. 24A illustrates high power photograph depicting a cluster of EGFP-positive cells migrating ventrolaterally, away from the third ventricle within the developing thalamus. One cell from this cluster (FIG. 24B) displays many of the classic features associated with newly-generated, migrating neurons including bipolar morphology and a leading process with apparent pseudopodia. FIG. 24C illustrates low power image depicting a pair of EGFP-positive cells which appear to be exiting the intermediate zone (iz) and entering the cortical plate (cp). DAPI stained nuclei are depicted in blue. These highly similar cells expressed EGFP (green) in their cell bodies as well as their pial directed, leading processes (arrowheads) (FIG. 24D). This pair also expressed HuC/D (red), a marker of newly generated, migrating and immature neurons, in their cell bodies and processes (FIG. 24E). Higher magnification of inset depicted on FIG. 24C is shown on FIG. 24F (Scale bar=10 um).

b) FIG. 25A depicts a 3D reconstruction of the E18 cortical plate derived from high power z series images. Numerous EGFP-positive cells (green) demonstrate morphologies and location consistent with differentiating neurons. FIGS. 25B and 25C show higher magnifications of insets illustrated on FIG. 25A. These cells were identified as neurons according to their co-expression of the neuronal class III beta-tubulin (red). DAPI stained nuclei are shown in blue. A similar 3D reconstruction is shown on FIG. 25D. Higher magnification of insets illustrated on FIG. 25D is shown on FIG. 25E and FIG. 25F. (Scale bar=50 um).

### Example 39

In vivo injection followed by electroporation of mouse ventricular zone cells at P0 with HIV-EGFP Numblike versus HIV-EGFP control vector.

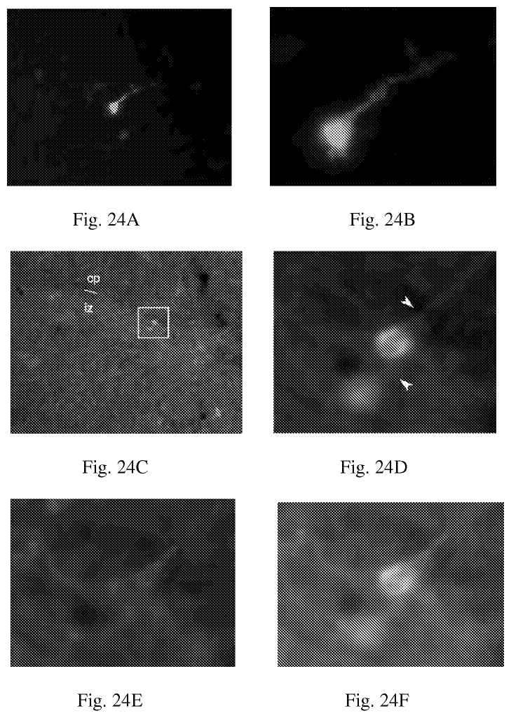

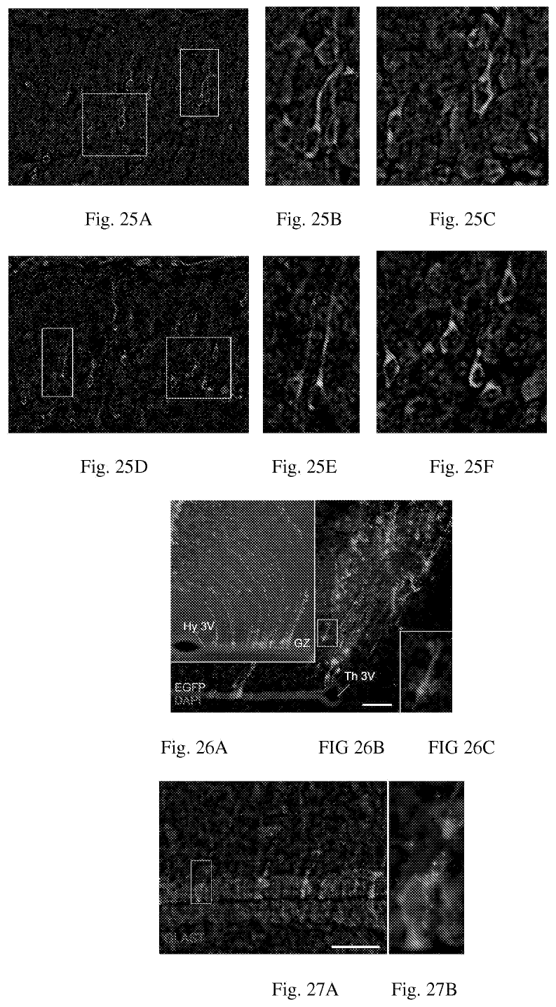

Upper left corner of FIG. 26A shows a low power image of the hypothalamic third ventricle (Hy 3V) rotated so that the electroporated portion of the ventricular neuroepithelium is upwards, and the superior portion of the ventricle is to the right. Radially-oriented, EGFP-positive cells (radial glia transfected with control) are seen lining the ventricle and represent the majority of cells labeled by control (˜80%). Their long EGFP-positive processes are observed to extend to the pia within the plane of section. A smaller proportion of cells were located at or near the pial margin-always closely associated with labeled radial processes. Middle section (FIG. 26B) depicts cells transfected with HIV-EGFP-Numblike at P0. Forty-eight hours later, virtually all of the cells have migrated away from the ventricle consistent with their new identity as differentiating neurons. Many of the cells dispersing away from the ventricle showed morphologies and trajectories consistent with the classical appearance of radially migrating neurons. Lower right corner of FIG. 26 (FIG. 26C) depicts an EGFP-positive cell with features characteristic of migrating neurons, including bipolar morphology, a thick leading process with pseudopodia, and a thin lagging process. Abbreviations: Th3V=thalamic portion of the third ventricle. FIG. 27A shows a 3D reconstruction derived from 180 high power, z-series images of the thalamic third ventricle. P2 germinal zone cells, including those transfected with control vector and displaying radial glial morphology, consistently expressed GLAST (glial glutamate transporter) in their cell membranes. FIG. 27B shows the radial glial cell depicted in the inset illustrated on FIG. 27A at higher magnification (scale Bars=50 um on FIG. 27B and 100 um on middle section of FIG. 27A).

### Example 40

Intraventricular injection of the HIV-EGFP-Numblike transfectant followed by in vivo electroporation upregulates Numb expression.

FIGS. 28A and 28B illustrates a 3D reconstruction depicting a section 50 um. EGFP labeled cells, both within and beyond the germinal zone, showed increased Numb immunoreactivity (red) relative to non-transfected cells in the same section (FIG. 28B). A portion of the germinal zone is shown at higher magnification in lower right corner on FIGS. 28A and 28B. The insets again show a relatively disorganized ventricular zone following transfection. This disorganization may have been related to the emigration of cells previously lining the ventricle, but might also reflect tissue injury due to electroporation alone (scale bar=100 um on FIGS. 28A and 28B and 100 um in lower right corner on FIGS. 24A and 24B).

### Example 41

In vivo injection of the HIV-EGFP-Numb^{PTB-/PRR-} transfectant followed by electroporation promotes neuronal differentiation in postnatal mice.

FIGS. 28C and 28D depicts a coronal section through dorsal neocortex in a P3 mouse transfected with the HIV-EGFP-Numb^{PTTB-/PRR-} forty-eight hours earlier. Dapi-stained nuclei in the region of electroporation indicated large numbers of cells (arrows) migrating radially through the various layers of the cerebral cortex including the subventricular zone (SVZ), corpus callosum (CC), subplate (SP), and cortical plate (CP). FIG. 28D illustrates EGFP expressing cells (triangles) also appeared to migrate laterally in the intermediate zone (IZ) as is known to occur during normal development. Most EGFP-positive cells also expressed high levels of Hu C/D, indicating they were newly-generated neurons (not shown).

A 3D reconstruction from confocal z-series images shows HIV-EGFP-Numb^{PTB-/PRR-} transfected cells in the P3 thalamus (FIG. 28E). Most of the cells are located outside the germinal zone 48 hours later, and can be recognized as migrating neurons by their morphologies and increased expression of Hu C/D—having been induced to begin differentiating simultaneously following electroporation and to migrate as a cohort. Other cells nearer the ventricle show migratory profiles and appear to be exiting the VZ, but have not yet begun to express Hu C/D (arrowheads). Scale bar=100 um on FIG. 2A and 40 um on FIG. 28E.

### Example 42

Transiently expressed EGFP strongly correlates with markers of neuronal differentiation in cells transfected with Numblike following in vivo injection.

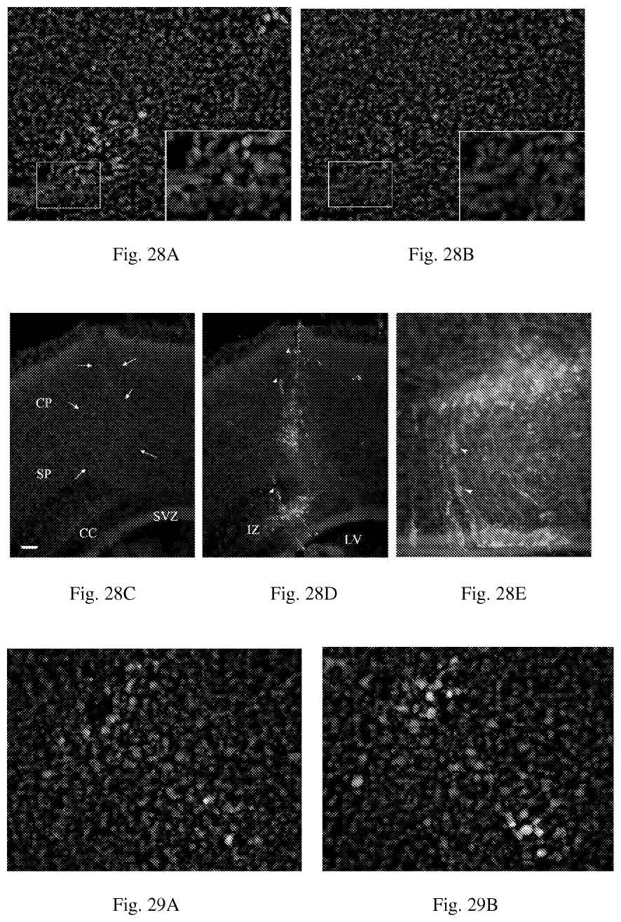

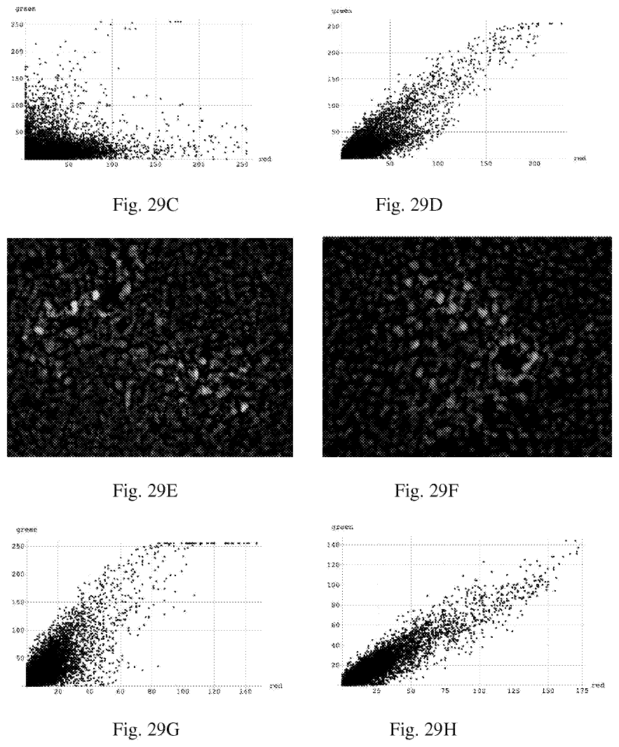

All EGFP positive cells were analyzed immunohistochemically. A discrete cluster of EGFP-positive cells located in the thalamus, 600-700 microns dorsolateral to the germinal zone is depicted. Consecutive sections containing cells from this cluster were stained for markers of neural differentiation including GLAST (FIG. 29A), Numb (FIG. 29B), TUJ (FIG. 29E), and DCX (FIG. 29F). Subsequent pixel-by-pixel analysis of these images demonstrated strong correlation between EGFP intensity (green) and markers of neuronal differentiation (red) (R-squared values ≥0.76). On the other hand, EGFP expression was not correlated with expression of the immature marker, GLAST (FIG. 26C: R-squared value=0.1079). DCX (FIG. 26H) showed the highest correlation (R-squared value=0.909), while Numb reactivity was also strongly correlated with EGFP expression in Numblike transfected cells (FIG. 29D: R-squared value=0.76). Brains of animals injected with transfectants and electroporated in vivo at PO were sectioned to completion and inspected microscopically. While large clusters of neurons transfected at PO with Numb or Numblike were detected within each of the brains, having migrated and differentiated as a cohort, they no longer expressed EGFP-evidence that the integrase deficient lentivectors remained episomal (did not integrate) and produced only transient transfection.

### Example 43

HIV-EGFP-Numb^{PRR-/PTB-} and HIV-EGFP-Numblike lentiviruses reduce proliferation and promote differentiation in Ras+, Breast cancer cells.

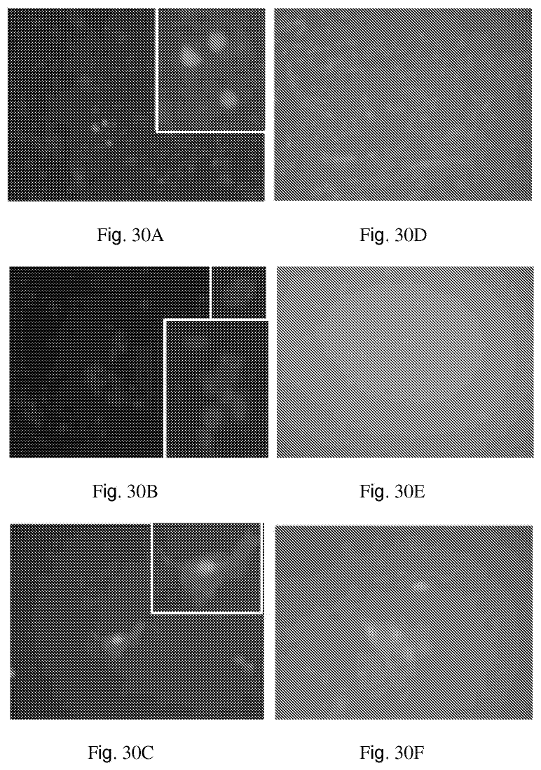

At 5 days post-transduction/post-plating, Ras+ cancer cells transduced with control HIV-EGFP lentivirus showed rapid proliferation and chaotic morphologies (FIG. 30A). The inset shows three round, brightly fluorescing cells whose appearance was consistent with cancer stem cells. In contrast, cells transduced with HIV-EGFP-Numb^{PRR-/PTB-} showed evidence of symmetrical, terminal divisions (cell pairs) on day 5, as well as reduced proliferation (FIG. 30B). In addition to blocking proliferation, transduction with HIV-EGFP-Numblike induced Ras+ cancer cells to adopt a phenotype consistent with normal breast epithelial cells (FIG. 30C). At 10 days post-plating/post-transduction, Ras+ cancer cells transduced with control virus fluoresced more brightly than on day 5, but otherwise, continued to show the disorganization characteristic of breast cancer cells, in vitro (FIG. 30D). In contrast, on day 10, few, mostly small cells were present in with HIV-EGFP-Numb^{PRR-/PTB-} transduced culture (FIG. 30E). Meanwhile, additional cells reverting to a normal, breast epithelial phenotype were identifiable in HIV-EGFP-Numblike transduced cultures (FIG. 30F).

### Example 44

Examples of expressed or targeted transgenes (and/or their corresponding proteins) utilized in the present invention.

Any transgene sequences (and/or their corresponding proteins) effective in fulfilling the present invention is suitable for use in the present invention. Suitable nucleotide sequences (and/or their corresponding proteins) may be drawn from any species so long as the desired cells or behavior is achieved. Likewise, the method of naming such sequences (or their corresponding proteins), either in lower case or upper case letters herein, does not necessarily imply a particular species. The sequences included in the accompanying sequence listing and stored in the NCBI database (listed by accession number) represent examples of sequences (and/or their corresponding proteins) referenced above in the present application. They are also examples of specific transgene encoding sequences (cds) suitable for use in the present invention, but do not in any way limit the practice of the invention. The cited references herein, to the extent that they provide exemplary procedural or other details supplementary to those set forth herein, are specifically incorporated herein by reference.

Likewise, the contents of all cited patents, patent applications, and publications are incorporated by reference in their entireties, including but not limited to US721124; U.S. Pat. Nos. 5,677,139; 6,432,711; 5,453,357; 5,593,875; 5,783,566; 5,928,944; 5,910,488; 05,824,547; 8,921,332; U.S. Pat. Application 20180028567; U.S. Pat. No. 5,925,565; U.S. Pat. No. 20180112235; U.S. patent Ser. No. 10/435,713; U.S. Pat. Nos. 8,110,360; 5,464,764; U.S. Pat. No. 20180053547; U.S. patent application Ser. No. 10/080,272; U.S. Pat. Nos. 6,485,961; 4,683,202; U.S. patent Ser. No. 13/902,444; U.S. Pat. No. 4,366,241; U.S. Pat. No. 20110236962; U.S. Pat. Nos. 5,627,059; 6,150,148; 6,074,605; 4,857,451; 6,204,061; U.S. Pat. No. 20160281047; U.S. Pat. No. 20040171156; U.S. Pat. No. 20120277120; U.S. Pat. No. 20180023045; U.S. Pat. Nos. 6,277,608; 6,773,669; 6,617,154; U.S. Pat. No. 20070231873; U.S. Pat. Nos. 7,422,889; 5,654,182; 8,569,041; 9,063,136; U.S. Pat. Application 20170258837; U.S. patent application Ser. No. 10/399,364; U.S. Pat. No. 20130196441; U.S. Pat. Nos. 6,074,605; 9,534,989; WO03/018751; U.S. Pat. Nos. 7,029,916; 3,996,345; 5,545,130; U.S. patent application Ser. No. 10/316,335; U.S. Pat. No. 20130253040; U.S. patent Ser. No. 12/421,352; U.S. Pat. Nos. 9,988,624; 4,661,913; U.S. Pat. Application 20120088842; U.S. Pat. No. 20110009807; U.S. Pat. Nos. 9,132,153; 5,134,070; 5,935,819; U.S. Pat. No. 20050118705; U.S. Pat. Nos. 7,141,425; 8,153,432; U.S. patent application Ser. No. 14/834,932; U.S. Pat. No. 3,850,752; U.S. patent application 20040214333; U.S. Pat. No. 20170316353; U.S. patent Ser. No. 10/017,760; U.S. Pat. No. 5,677,177; U.S. Pat. No. 20170218355; U.S. Pat. Nos. 6,090,617; 5,888,732; U.S. patent application Ser. No. 10/225,446; U.S. Pat. No. 20170307606; U.S. Pat. No. 20030104588; U.S. patent application 20040197883; U.S. Pat. Nos. 6,773,669; 8,667,840; 8,450,112; 9,546,350; U.S. patent application Ser. No. 10/781,440; U.S. Pat. No. 9,669,058; U.S. patent application Ser. No. 14/834,932; U.S. Pat. No. 8,677,839; U.S. Pat. No. 20110213288; U.S. Pat. Nos. 7,771,984; 7,991,559; U.S. Pat. No. 20060224192; U.S. Pat. Nos. 4,714,682; 8,332,160; 9,029,109; 7,771,984; U.S. Pat. No. 20140350456; U.S. Pat. No. 20110061474; U.S. Pat. Nos. 6,617,154; 4,833,080; 7,112,715; 3,817,837; 9,738,918; 6,746,441; 5,885,836; U.S. Pat. No. 20040115784; U.S. Pat. No. 20180179485; U.S. Pat. Nos. 9,593,359; 6,077,479; U.S. Pat. No. 20160018427; U.S. patent application Ser. No. 10/225,446; U.S. Pat. Nos. 5,676,646; 4,220,916; U.S. patent application Ser. No. 10/080,272; U.S. Pat. No. 5,098,843; U.S. Pat. No. 20170283761; U.S. Pat. Nos. 5,612,207; 6,509,156; U.S. Pat. No. 20110082093; U.S. Pat. No. 4,275,149; U.S. patent application Ser. No. 10/675,592; U.S. Pat. Nos. 4,277,437; 8,450,112; U.S. Pat. No. 20160298074; U.S. Pat. No. 20120156786; U.S. Pat. No. 5,487,992; U.S. patent application Ser. No. 12/421,352; U.S. patent Ser. No. 10/443,074; U.S. patent application Ser. No. 10/675,592; U.S. Pat. No. 20030073238; U.S. Pat. No. 20150297887; U.S. patent application Ser. No. 11/127,557; U.S. Pat. Nos. 8,932,850; 8,697,359; 6,773,669; U.S. Pat. Application No. 20030059945; U.S. Pat. No. 20110065171; U.S. Pat. No. 7,186,559; U.S. patent application Ser. No. 13/902,444; U.S. Pat. Nos. 5,478,722; 7,186,559; 8,726,744; U.S. patent application Ser. No. 10/781,440; U.S. Pat. No. 9,361,427; U.S. Pat. No. 20070105206; U.S. Pat. No. 6,774,279; U.S. Pat. No. 20180169148; U.S. Pat. No. 7,141,425; U.S. Pat. No. 20160333302; U.S. Pat. No. 20170029805; U.S. Pat. No. 20080138877; U.S. Pat. Nos. 5,720,921; 4,498,766; 8,758,623; 6,143,527; 5,631,153; U.S. Pat. No. 20050064584; U.S. Pat. Nos. 4,989,977; 6,090,617; 5,160,974; 4,767,206; 6,090,617; 8,677,840; 8,584,535; U.S. Pat. Application 20200237825; U.S. Pat. Nos. 4,284,412; 9,132,153; 5,928,906; 9,790,490; 8,110,122; PCT/US2014/028561; U.S. Pat. application 20180112235; U.S. Pat. Nos. 3,826,364; 6,916,632; 4,774,189; U.S. Pat. No. 20180028567; U.S. Pat. No. 20070249036; U.S. Pat. Nos. 7,029,916; 8,584,536; 9,982,279; U.S. Pat. No. 20160272961; U.S. Pat. Nos. 8,450,112; 6,956,146; WO 2004/031353; U.S. Pat. Nos. 6,482,619; 6,689,610; 3,939,350; 9,896,696; 4,946,793; U.S. Pat. No. D731634; U.S. Pat. No. 20170067007; U.S. Pat. Nos. 5,137,817; 6,654,636; U.S. Pat. No. 20140121728; and U.S. Pat. No. 4,959,317.

### Example 45

| Names | total | Elements |
|---|---|---|
| Human Nanog | 4 | PPP1R14A DNMT3B ZFP42 EPCAM |
| Human Oct4 | | |
| Mouse Oct4 | | |
| Human Nanog | 65 | CDH3 ESRP1 PRSS16 KRT19 OSBPL10 GLB1L3 LECT1 POU5F1P4 COBL |
| Human Oct4 | | C6orf132 CD24 SMPDL3B HEY2 SCNN1A DMKN MARVELD3 LINC00545 |
| | | KRTCAP3 PDLIM1 GLI1 FOXH1 EPHX3 SEPHS1 TMEM30B ESRP2 FLNB APOE |
| | | CLDN10 F11R GCNT2 CDH1 PODXL OVOL2 KLRG2 SLC7A3 ESRG RBP7 |
| | | RASGEF1A DPPA4 LIN28A PRR15 RBPMS2 MARVELD2 SPINT2 SCGB3A2 |
| | | TMEM125 IRF6 KRT18 ADD2 FOXA3 TMEM37 POU5F1P3 HLA-DPB2 POU5F1B |
| | | C1orf172 CLDN7 NMRK2 EPHA1 GRHL2 ABPMS ARHGAP6 TDGF1 ZSCAN10 |
| | | KCTD14 BAIAP2L1 |
| Human Oct4 | 1 | IGF2BP1 |
| Mouse Oct4 | | |
| Human Nanog | 24 | CR2 EPB41L4B PACSIN3 TC2N FLT1 CPVL FERMT1 LOC647859 POU5F1 |
| | | LOC100506013 UST JPH1 GRTP1 Human NANOG RBM47 EPB41L5 GPR160 |
| | | TSTD1 DSG2 C9orf135 BEND4 MAL2 CCDC172 ELOVL7 |
| Human Oct4 | 16 | TMEM108 PRDM14 FAM124A NANOG FAM46B TLCD1 ERBB3 PPAP2C RAB25 |
| | | CKMT1A KCNS3 Human POU5F1 VRTN ARHGEF5 COX6A1 CTSC |
| Mouse Oct4 | 150 | CLPB TSPAN1 PCBD1 MTF2 ACVR2B DHCR24 SLC7A7 DDT RCOR2 NMB |
| | | ICOSL WOR5 FABP3 CUL4B TUBA4A RLBP1 BHMT2 MSC AMN CLGN UGT8A |
| | | H2-OA FTHL17 PARK7 RPS6KA1 UTF1 CYB561 NGRN CLDN4 CIRBP ZC3H10 |
| | | CKMT1 PEX7 GNPAT ADCK3 PLA2G1B GNG3 PDCD4 RHD PLA2G5 KLK1 |
| | | EIF2S2 SLC15A1 TSPAN13 CIDEA TRIM13 TEX19.1 SLC27A2 RIPK4 |
| | | MPHOSPH8 GM9835 IMPA2 GRHPR PLEKHB1 MSH6 DPPA5A AIRE DAZL |
| | | PRSS8 GNG13 DOX25 SIN3A PKP3 SH3GL2 NTHL1 MTIF2 TFCP2L1 TERF1 |
| | | MFNG RHOX6 DKKL1 ULK1 CAR4 1810019J16RIK MOUSE POU5F1 FEM1B |
| | | QPRT MKRN1 SCT TFAP2C TEX14 COX8A POU2F1 TRP53INP1 FGFR4 GPA33 |
| | | PPP1R14D MORC1 LAMA1 TESC ORC5 ABCC5 REC8 NR0B1 APOBEC2 |
| | | SPINT1 ZMAT5 RASGRP2 GAB1 SNRPN NFU1 CELSR1 CEACAM10 TEX11 |
| | | GAD1 ACOT8 TNFRSF13C JPH3 FGD1 PMAIP1 EXOSC5 MYCN EIF4EBP1 |
| | | MEP1B PDXP LDHC SLC47A1 PSRC1 GLDC CHGA NIT1 CLDN6 ABTB1 PAX8 |
| | | RP9 PIM2 SPA17 APOC1 PYCARD TRAP1 CENPM GJB3 GTSF1L SOX2 |
| | | PHLDA2 2210409E12RIK MSH2 ST14 APLP1 OSGEP PNKD FXR1 NUP210 |
| | | RDM1 RFX2 USP48 TULP2 COX7A1 INPP5D SLC44A4 |

1. A method for developmentally-activating a cell, said method comprising the steps of selecting a non-pluripotent somatic cell and exposing said cell to an electric field, whereby said cell shows changes in global gene expression consistent with developmental activation characterized by one or more of a. rapid induction to pluripotency (within 24-72 hours); b. induced expression of a pluripotency or totipotency reporter gene; c. enrichment of pluripotency or totipotency associated gene expression; d. adoption of cell morphology consistent with pluripotency or totipotency; e. embryoid formation or embryo formation consistent with pluripotency or totipotency; f. hierarchical clustering with or among pluripotent or totipotent cells relative to somatic cells in Heatmaps; g. clustering with or among pluripotent or totipotent cells relative to somatic cells in Principal Component Analysis plots; and h. increased expression of nucleic acids enriched in pluripotent or totipotent cells.

2. The method of claim 1 wherein the selected cells are a. isolated from cell culture; b. autologous; c. heterologous; d. exogenous; e. exogenous; f. derived from a donor; g. genetically modified; h. subject to genetic correction; i. harboring a mutation; j. euploid; k. obtained from a patient; l. obtained from a donor; m. non-immortalized; or n. obtained by thawing.

3. The method of claim 1 wherein the selected cells overexpress nucleic acid(s) or protein(s) corresponding to one or more transcription factor or other cell fate determinant enriched in pluripotent or totipotent cells.

4. The method of claim 3 wherein said nucleic acids or proteins include one or more corresponding to ZCAN4, PPP1R14A, DNMT3B, ZFP42, EPCAM, OCT4, CXCR4, SOX2, PRR+ NUMB, NOTCH, NANOG, HOXB4, and/or a gene with LIF activity.

5. The method of claim 1 wherein the nucleic acids overexpressed in or introduced to said cells correspond to one or more selected from miR-302/367 cluster small RNAs (miR-302a, miR-302b, miR-302c, miR-302d, miR-367), miR-371-373 cluster small RNAs (miR-371, miR-372, miR-373), miR-17-92, C 19MC cluster members, miR-133b, miR 200a, miR 23a, and miR 743b-5p, miR-187, miR-299-3p, miR-499-5p, miR-628-5p, miR-888, let-7 (let-7-b,e,f,g), miR-30 (miR-30-a-e), the mouse miR-290-295 cluster small RNAs (miR-290, miR-291a-3p, miR-291b, miR-292, miR-294, miR-295, miR-29, miR-296, miR-106a cluster, miR138, miR130, miR-301, miR-721, and miR-93, further promoting induced potency in said cells.

6. The method of claim 1 wherein the selected cells are incubated in a growth medium.

7. The method of claim 1 wherein nucleic acids and/or proteins introduced to or overexpressed in said selected cells are selected from the group comprising a short Numb isoform, Numblike, MyoD, myogenin, Myocardin, Ifrd1, Myf5, Myf6, Mef2c, Mef2a, Mef2c, Tbx5, JAK inhibitor I, Nkx2.5, Esrrg, Mesp1, Zfpm2 Ets2, Mesp, Myocd, Nkx2.5, Hand2, Gata 4, Gata 5, and Gata 6, Sox9, CREB-binding polypeptide, Runx2, HNF-1, HNF-3, HNF-4, HNF-6, Cebpa, Cebpb, Atf5, Prox1, Foxa3, Foxa1, Foxa2, Lmx1a, Lmx1b, Brn2, Otx2, Nurr1, REN, Neurogenin1, Neurogenin2, Neurogenin3, Mash 1 (Asc11), Phox2a, Phox2b, dHand, Brn2, Myt1, Gata3, Shh, FGF8, Lmx1b, Nkx2.2, Pet1, Lbx1, Rnx, PITX2, Dlx2, Dlx5, REN, Ngn2, Ptx-3, Gata2, REST4, Foxa2, Sox17, Mafa, HLXB9, Runx1/AML, ERG, GATA2, LMO2, RUNX1c, Nov(Ccn3), SCL, Runit1, Hlf, Prdm5, Pbx1, Zfp37, Mycn, Meis1, FOSB, GFI1, SPI1 Pdx1, Olig1, Olig2, Zfp536, Nkx2.2, Nkx6.2, Sox10, ST18, Gm98, Myt1, Nov(Ccn3), Foxa1, Er7l, Klf2, Tal1, Zfp488, Six1, Six2, Osr1, Eya1, Hoxal1, Snai2, PRDM16, Nfia, Nfib, Etv2, Fli1, Erg1, Klf2, Lmo2, Mitf, Sox10, Pax3, Hes1, Id1, Pax6, Brn4, Klf4, E47; NeuroD1, Neurod2, Mytl1, Lhx3, Hb9, Isl1, Adam3, Akap3, Aurkc Bmp15, Fig1a, Figalpha, Daz1, Stra8, Fox12, Oogenesin1, Oogenesin2, Oogenesin3, Oogenesin4, Sycp2, Sycp3, Spol1, Rec8, Dmc1, Mos, Stag3, Ccnb1, Foxa1, Foxo3, Foxr1, Sohlh1, Sohlh2, Nobox, Obox1, Obox2, Obox3, Obox4, Obox6, Oaz3, Otx2, Ldhc, Lhx3, Lhx8, Lhx9, Oog1, Sp1, Zfp38, Trf2, Tb2/trf3, Taf4b, Taf7l, Taf7l, Tia1, Phtf1, Tnp2, Hils1, Daz1, Bmp15, Pttg3, Aurkc, Otx2, Sox15, Sox30, Foxr1, Alf, Oct4, Dppa3/stella, Zfp38, Rps6ka3, Hinfp, Npat, Sp1, Sp3, Hoxa1, Hoxa7, Hex, Yp30, Zp1, Zp2, Zp3, Sfe1, Sfe9, Opo, Pln, Rdv, Gld1, Clgn, Tekt1, Fscn3, Dnahc8, Gdf9, And Pttg3, or Hes1, thereby promoting differentiation to a desired differentiating cell type.

8. The method of claim 1 wherein nucleic acids introduced to or overexpressed in said selected cells are selected from the group comprising miR-124, miR-125b, miR-128, miR-1-1, miR-1-2, miR-1, miR-1-1, miR-1-2, miR-9/9, miR-206, miR-26a, miR-133, miR-133 a-1, miR-133 a-2, miR-208, miR-499, MMU-MiR351, M MU-MiR615, MMU-MiR592, MMU-MiR882, MMU-MiR185, MMU-MiR491, MMU-MiR326, MMU-MiR330, MiR212, miR-128, miR-181, miR-16, miR-103, miR-107, miR-150, miR-181, miR-155, miR-24, miR-17, miR-16, miR-103, miR-107, miR-150, miR-155, miR-221, miR-222, miR-451, miR-16, miR-24, miR-17-5p, miR-20a, miR-106a, miR-16, miR-103, miR-107, miRNA-155, miR-24, miR-17, miR-223, miR-16, miR-103, miR-107, miR-155, miR-24, miR-17, miR-125b, miR-26a, miR-203, miR-302, miR-302-367, thereby promoting differentiation to a desired differentiating cell type.

9. The method of claim 1, characterized in that said cells and/or their progeny are incubated in a differentiation medium comprising one or more agents selected from the group comprising:
a. retinoic acid, neurotrophin 3 (NT3), nerve growth factor (NGF), glial cell-line derived growth factor (GDNF), and/or interferon-γ (IFN-γ), hexamethylene bis acrylamide, dimethylsulfoxide (DMSO), fetal bovine serum (FBS), normal bovine serum (NBS), retinoic acid, cardiomyocyte conditioned medium, vascular endothelial growth factor (VEGF), LIF, thrombopoietin, a colony stimulating factor, M-CSF (CSF-1), GMCSF, IL-7, a cytokine promoting CD4+ T cell differentiation, sodium butyrate, activin A, epidermal growth factor, basic fibroblast growth factor, noggin, insulin-like growth factor and nicotinamide;
b. retinoic acid, neurotrophin 3 (NT3), nerve growth factor (NGF), glial cell-line derived growth factor (GDNF) and/or interferon-γ (IFNγ);
c. hexamethylene bis acrylamide or dimethylsulfoxide;
d. dimethylsulfoxide, retinoic acid and/or cardiomyocyte conditioned medium;
e. thrombopoietin and colony stimulating factors appropriate to the desired cell type;
f. LIF, neurotrophin 3 (NT3), and/or nerve growth factor (NGF);
g. a colony stimulating factor, optionally M-CSF (CSF-1), GM-CSF, IL-7, or a cytokine promoting CD4+ T cell differentiation; and
h. sodium butyrate, activin A, retinoic acid, epidermal growth factor, basic fibroblast growth factor, noggin, insulin-like growth factor II and nicotinamide.
i. a serum free medium

10. The method of claim 1, characterized in that the selected cell is modified to express
a. telomerase altering the cell's life span;
b. a gene product deficient in a patient;
c. a nucleic acid sequence encoding a protein selected from ASP, HRPT,
HTT, NPC1, GBA, PARK2, VHL, HEXA, HEXB, HRPT, HTT, GLA, HBB, NPC1, PARK2, SNAP29, BCS1L, ALAS2, ESCO2, IDS, RP1, RP2, RPGR, PRPH2, IMPDH1, PRPF31, CRB1, PRPF8, TULP1, CA4, HPRPF3, ABCA4, EYS, CERKL, FSCN2, TOPORS, SNRNP200, PRCD, NR2E3, MERTK, USH2A, PROM1, KLHL7, CNGB1, TTC8, ARL6, DHDDS, BEST1, LRAT, SPARA7, CRX, NODAL, NKX2-5, ZIC3, CCDC11, CFC1, SESN1, CLCN5, OCRL, AASDHPPT, ATP7B, MEFV, ABCA1, MSTN, C2ORF37, FECH, FGFR2, IGF-2, CDKN1C, H19, KCNQ1OT1, BTD, BCS1L, FLCN, ATP2A1, NOTCH3, HPS1, HPS3, HPS4, HPS5, HPS6, HPS7, AP3B1, SCN1A, SCN2A, ABCC6, WT1, ZEB2, LMNA, PMP22, MFN2, PSEN1, PSEN2, APP, APOEÎμ4, ALAD, UBE3A, FGFR2, VPS33B, ATM, PITX2, FOXO1A, FOXC1, PAX6, DMPK, CNBP, FVIII, KRT5, KRT14, DSP, PKP1, JUP, PLEC1, DST, EXPH5, TGM5, LAMA3, LAMB3, LAMC2, COL17A1, ITGA6, ITGA4, ITGA3, COL7A1, FERMT1, MYO5A, RAB27A, MLPH, RYR1, TP53, NTRK1, GALNS, GLB1, AP4M1, AP4S1, AP4B1, AP4E1, PTEN, PDGFRB, BCKDHA, BCKDHB, DBT, DLD, GALT, GALK1, GALE, COL2A1, PMP22, APC, TCF4, STK11, MYO7A, USH1C, CDH23, PCDH15, USH1G, USH2A, GPR98, DFNB31, CLRN1, DHCR7, PROS1, ASPM, AGXT, GRHPR, DHDPSL, MEN1, RAG1, RAG2, GLA, LYST, PKD1, PKD2, HPRT, RELN, MMAA, MMAB, MMACHC, MMADHC, LMBRD1, MUT, FGFR3, DNAI1, DNAH5, TXNDC3, DNAH11, DNAI2, KTU, RSPH4A, RSPH9, LRRC50, UROD, TBX4, MSH2, MLH1, MSH6, PMS2, PMS1, TGFBR2, MLH3, RAB23, CREBBP, COL11A2, ATP2A2, RPS6KA3, CFTR, ATP7A, COL1A1, COL1A2, COL3A1, COL5A1, COL5A2, TNXB, ADAMTS2, PLOD1, B4GALT7, DSE, FBN1, MR1, CPDX, SGSH, NAGLU, HGSNAT, GNS, GLDC, AMT, GCSH, ERCC6, ERCC8, ABCA12, FGFR2, ASPA, CBS, GBA, PANK2, EXT1, EXT2, EXT3, DYSF, TIA1, GNE, MYH7, Titin, MYOT, MATR3, GCDH, ETFA, ETFB, ETFDH, SMPD1, NPA, NPB, NPC1, NPC2, 21, FGFR3, IDUA, MYCN, HSPG2, MECP2, PPDX, IKBKG, AAAS, FGD1, EDNRB, CP, LMBR1, COL2A1, FGFR3, HMBS, ADSL, GUSB, HDAC8, SMC1A, NIPBL, SMAS, RAD21, PC, TCOF1, POLR1C, or, POLR1D), HGD, COL4A3, COL4A4, COL4A5, ATP1A3, C9orf72, SOD1, FUS, TARDBP, CHCHD10, MAPT, ALMS1, ENG, ACVRL1, MADH4, HTT, ATXN1, ATXN2, ATXN3, PLEKHG4, SPTBN2, CACNA1A, ATXN7, ATXN8OS, ATXN10, TTBK2, PPP2R2B, KCNC3, PRKCG, ITPR1, TBP, KCND3, FGF14, CHD7, ABCD1, JAG1, NOTCH2, TP63, TREX1, RNASEH2A, RNASEH2B, RNASEH2C, SAMHD1, ADAR, IFIH1, GFAP, ARSB, PRNP, EMD, LMNA, SYNE1, SYNE2, FHL1, TMEM43, PDS, AASS, FGFR1, FGFR2, HTRA1, COL11A1, COL11A2, COL2A1, COL9A1, 11p15, FANCA, FANCB, FANCC, FANCD1, FANCD2, FANCE, FANCF, FANCG, FANCI, FANCJ, FANCL, FANCM, FANCN, FANCP, FANCS, RAD51C, XPF, RET, GCH1, PCBD1, PTS, QDPR, MTHFR, DHFR, IKBKAP, ATP13A2, MED12, PAX6, ATP2C1, GNE, MYHC2A, VCP, HNRPA2B1, HNRNPA1, COL1A1, COL1A2, IFITM5, COH1, ALDH3A2, FXN, AP1S1, PAX3, MITF, WS2B, WS2C, SNAI2, EDNRB, EDN3, SOX10, VHL, GALL, RAB3GAP, ABCB7, SLC25A38, GLRX5, 5q, HFE, HAMP, HFE2B, TFR2, TF, CP, HEXA, TSC1, TSC2, PAH, HYAL1, DMD, HEXB, ERCC, RUNX2, HSPB8, HSPB1, HSPB3, GARS, REEP1, IGHMBP2, SLC5A7, DCTN1, TRPV4, SIGMAR1, FGFR2, FGFR3, ABCA4, CNGB3, ELOVL4, PROM1, ALS2, PROC, INPP5E, TMEM216, AHI1, NPHP1, CEP290, TMEM67, RPGRIP1L, ARL13B, CC2D2A, OFD1, TMEM138, TCTN3, ZNF423, AMRC9, LCAT, COL11A1, COL11A2, COL2A1, PTPN11, KRAS, SOS1, RAF1, NRAS, HRAS, BRAF, SHOC2, MAP2K1, MAP2K2, CBL, PHF8, PEX1, PEX2, PEX3, PEX5, PEX6, PEX10, PEX12, PEX13, PEX14, PEX16, PEX19, and PEX26.
d. a T cell receptor
e. an antibody
f. a T cell receptor and an antibody

11. The method of claim 1, characterized in that the selected cell or its progeny is modified to expresses:
a. a beneficial nucleic acid sequence;
b. a synthetic oligonucleotide, complementary RNA (siRNA), microRNA, short-hairpin (shRNA), interfering microRNA, antisense RNA;
c. a nucleic acid sequence or protein retarding viral infection;
d. a nucleic acid sequence or protein rendering said cells and/or their progeny less capable of sustaining viral replication;
e. a nucleic acid sequence or protein rendering said cells and/or their progeny less capable of sustaining viral transcription;
f. a synthetic oligonucleotide directed against an immunodeficiency virus co-receptor;
g. a decoy RNA;
h. an HIV psi sequence;
i. a nucleic acid or protein sequence rendering said cells and/or their progeny disease resistant.

12. The method of claim 1, characterized in that wherein a protein, nucleic acid or agent is present in order to screen for its ability to induce phenotypic changes or differentiation of the selected cells into desired cell populations.

13. The method of claim 1, characterized in that magnetoporation, electroporation, liposomes, nanocapsules, nanovaults, a vector that does not integrate, and/or another approach avoiding retroviral/lentiviral integration or other random alteration of said cell's genome is utilized, including site-directed mutagenesis.

14. The method of claim 1 further comprising site directed mutagenesis effected by CRISPR/Cas9.

15. The method of any of claims 1 to 14, characterized in that said cell and/or its progeny are cultured in a three-dimensional or two-dimensional scaffolding, said cells and/or its progeny are grown in a de-cellularized organ or on de-cellularized tissue, or said cells or its progeny are utilized in conjunction with 3D printing or inkjet style printing technology for tissue engineering.

16. The method of claim 1 performed in vivo.

17. A cell population produced by a method of anyone of claims 1-16.

18. An electroporation/magnetoporation catheter suitable for performing the method of claim 16.

19. The cell population of claim 17 for use in treating a patient with a condition or disease:
a. ameliorated by correction of a genetic deficiency;
b. dependent upon expression of genes producing susceptibility;
c. ameliorated by renewed cell proliferation; or
d. characterized by dysplasia, cancer or aberrant cell behavior.

20. The method of claim 1, wherein selected cells are assessed according to expression of a pluripotency or totipotency gene, a marker gene, a reporter gene, a transgenic marker, a marker gene encoded by a transgene expressing vector, an antibiotic resistance gene, a fluorescent protein, a reporter gene under the control of a cell type specific promoter, a reporter gene under the control of a pluripotency or totipotency activated promoter.

21. A cell culture dish with multiple compartments that is suitable for application of the electric field according to the method of claim 1.

22. The method of claim 1 wherein a. the electric field is applied via static, pulsatile electroporation, poration occurs mainly at the poles of the cell membrane, the voltage applied is ˜100 to 1300V, the pulse number is ˜10 to 1000 pulses, and/or the pulse length is ˜2 ms to 1,000 ms; b. the electric field is applied during flow electroporation, a constant voltage is applied across a fluidic channel, the channel varies in cross sectional area yielding varying field intensity, and/or poration occurs throughout the cell membrane; or c. the electrical field is induced by a magnetic field generator resulting in magnetoporation.

23. Use of a cell population of claim 17 in the preparation of a pharmaceutical composition for the treatment of a patient.

24. Method, cell population, electroporation/magnetoporation catheter, or cell culture dish of the preceding claims and as described in description and illustrated by drawings.
